(19) United States
(12) Patent Application Publication
(10) Pub. No.: US 2022/0380757 A1
(43) Pub. Date: Dec. 1, 2022

(54) BCL11A GUIDE AND BASE EDITOR DELIVERY

(71) Applicant: THE CHILDREN'S MEDICAL CENTER CORPORATION, Boston (US)

(72) Inventors: Daniel E. BAUER, Cambridge, MA (US); Jing ZENG, Brookline, MA (US)

(73) Assignee: THE CHILDREN'S MEDICAL CENTER CORPORATION, Boston, MA (US)

(21) Appl. No.: 17/764,413

(22) PCT Filed: Sep. 29, 2020
(86) PCT No.: PCT/US2020/053212

§ 371 Date: Mar. 28, 2022

Related U.S. Application Data
(60) Provisional application No. 62/908,094, filed on Sep. 30, 2019.

(51) Int. Cl. C12N 15/11; C12N 9/22; C12N 15/90

(52) U.S. Cl. CPC C12N 15/11; C12N 9/22; C12N 15/907; C12N 2310/20; C12N 2800/80

(57) ABSTRACT

Provided herein are 2′-O-methyl 3′phosphorothioate (MS)-modified synthetic nucleic acid molecules (single guide RNAs (sgRNAs)) in combination with a base editor as a ribonucleoprotein (RNP) complex for an electroporation-based ex vivo targeted gene disruption of the BCL11A erythroid enhancer's +55, +58, or +62, functional regions. Also provided herein are methods relating to the treatment of hemoglobinopathies by reinduction of fetal hemoglobin levels.

### CROSS-REFERENCE TO RELATED APPLICATIONS

This application claims benefit under 35 U.S.C. § 119(e) of U.S. Provisional Application No. 62/908,094 filed Sep. 30, 2019, the contents of which are incorporated herein by reference in their entirety.

### GOVERNMENT SUPPORT

This invention was made with Government support under Grant No. HL053749 awarded by the National Institutes of Health. The Government has certain rights in the invention.

### BACKGROUND

Normal adult hemoglobin comprises four globin proteins, two of which are alpha (α) proteins and two of which are beta (β) proteins. During mammalian fetal development, particularly in humans, the fetus produces fetal hemoglobin, which comprises two gamma (γ)-globin proteins instead of the two β-globin proteins. During the neonatal period, a globin switch occurs, referred to as the “fetal switch”, at which point, erythroid precursors switch from making predominantly γ-globin to making predominantly β-globin. The developmental switch from production of predominantly fetal hemoglobin or HbF (a2y2) to production of adult hemoglobin or HbA (α₂β₂) begins at about 28 to 34 weeks of gestation and continues shortly after birth until HbA becomes predominant. This switch results primarily from decreased transcription of the gamma-globin genes and increased transcription of beta-globin genes. On average, the blood of a normal adult contains less than 1% HbF, though residual HbF levels have a variance of over 20 fold in healthy adults and are genetically controlled.

Hemoglobinopathies encompass a number of anemias of genetic origin in which there is a decreased production and/or increased destruction (hemolysis) of red blood cells (RBCs). These also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickle) hemoglobin (HbS). HbS is prone to polymerization, particularly under deoxygenated conditions. HbS RBCs are more fragile than normal RBCs and undergo hemolysis more readily, leading eventually to anemia.

Recently, the search for treatment aimed at reduction of globin chain imbalance or predisposition to hemoglobin polymerization in patients with β-hemoglobinopathies has focused on the pharmacologic manipulation of fetal hemoglobin (α2γ2; HbF). The therapeutic potential of such approaches is indicated by observations of the mild phenotype of individuals with co-inheritance of both homozygous β-thalassemia and hereditary persistence of fetal hemoglobin (HPFH), as well as by those patients with homozygous β-thalassemia who synthesize no adult hemoglobin, but in whom a reduced requirement for transfusions is observed in the presence of increased concentrations of fetal hemoglobin. Furthermore, it has been observed that certain populations of adult patients with 62 chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease. It is now accepted that hemoglobin disorders, such as sickle cell anemia and the β-thalassemias, are ameliorated by increased HbF production.

The switch from fetal hemoglobin to adult hemoglobin (α2γ2; HbA) usually proceeds within six months after parturition. However, in the majority of patients with β-hemoglobinopathies, the upstream y globin genes are intact and fully functional, so that if these genes become reactivated, functional hemoglobin synthesis could be maintained during adulthood, and thus ameliorate disease severity. Unfortunately, the in vivo molecular mechanisms underlying the globin switch are not well understood.

Evidence supporting the feasibility of reactivation of fetal hemoglobin production comes from experiments in which it was shown that peripheral blood, containing clonogenic cells, when given the appropriate combination of growth factors, produce erythroid colonies and bursts in semisolid culture. Individual cells in such colonies can accumulate fetal hemoglobin (HbF), adult hemoglobin (HbA) or a combination of both. In cultures from adult blood, nucleated red cells accumulate either HbA (F-A+) only, or a combination of HbF and HbA (F+A+). Importantly, individual colonies contain both F+ and F-cells, indicating that both types are progeny from the same circulating stem cells. Thus, during the early stages of development in culture, cells execute an option, through currently unknown mechanisms, whether or not to express HbF. The proportion of adult F+ cells developing in culture does not appear to be preprogrammed in vivo, but appears to depend on culture conditions: A shift into the combined HbF and HbA expression pathway can, for example, be achieved in vitro by high serum concentrations, due to the activity of an unidentified compound that can be absorbed on activated charcoal.

Genome editing of autologous hematopoietic stem cells (HSCs) is a promising strategy to enable cure of blood disorders like β-hemoglobinopathies. A distal regulatory region upstream of the BCL11A gene regulates the expression of the BCL11A protein. The BCL11A protein acts as a stage specific regulator of fetal hemoglobin expression by repressing γ-globin induction. This upstream distal regulatory region mapped to the human chromosome 2 at location 60,716,189-60,728,612 in the human genomic DNA according to UCSC Genome Browser hg 19 human genome assembly. Noticeably, this upstream distal regulatory region consistently contains three DNAse 1-hypersensitive sites (DHS) +62, +58, and +55. Identification of these specific functional regions within this ˜12 kb molecules that play a role in the globin switch is important for the development of novel therapeutic strategies that interfere with adult hemoglobin and induce fetal hemoglobin synthesis. Previously it has shown that core sequences at the erythroid enhancer of BCL11A are required for repression of HbF in adult-stage erythroid cells but dispensable in non-erythroid cells. Prior efforts to use Cas9 and other programmable nucleases to edit human hematopoietic stem and progenitor cells (HSPCs) have shown variable efficiency, genotoxicity, requirement for HSC selection or expansion prior to infusion into recipient host, and limited ability to support long-term multilineage reconstitution with persistence of edited alleles. Provided here is an improved method of genome editing of the BCL11A's DHS +62, +58, and +55 functional regions, and the related reagents thereof The improved method comprises selection-free conditions for base editor:sgRNA ribonucleoprotein (RNP) electroporation of β-hemoglobinopathy patient-derived HSCs that induce highly efficient on-target editing, no off-target editing while lacking detectable genotoxicity.

### SUMMARY

Described herein is the use of base editing technology to produce genetic modification of BCL11A enhancer sequences without double strand breaks to induce high levels of fetal hemoglobin in erythroid cells derived from modified human hematopoietic stem and progenitor cells. Prior studies by the inventors and others have targeted these sequences by nuclease gene editing, but presented herein is a novel use of base editing to target these sequences. Work presented herein show that a single therapeutic base edit of the BCL11A enhancer is sufficient to prevent sickling and ameliorate globin chain imbalance in erythroid progeny from sickle cell disease (SCD) and βeta-thalassemia patient derived HSPCs respectively.

In addition, it is demonstrated herein that highly efficient multiplex base editing in human hematopoietic stem and progenitor cells to yield simultaneous and complementary genetic modification at multiple gene targets such as the BCL11A enhancer and a beta-thalassemia beta-globin gene mutation. It was found that base edits may be efficiently produced in multilineage-repopulating self-renewing human HSCs as assayed in primary and secondary recipient animals. Compared to non-engrafting progenitor cells, quiescent HSCs favor C to T editing. Base editing of the BCL11A enhancer results in potent HbF induction in vivo. Together, these results demonstrate the potential of RNP base editing of human HSPCs as a feasible alternative to nuclease editing for HSC-targeted therapeutic genome modification.

Accordingly, one aspect described herein provides a ribonucleoprotein (RNP) complex comprising: (a) a base editor protein; and (b) a nucleic acid sequence shown in Table 1, SEQ ID NOS: 1-139, wherein there is at least one chemical modification to a nucleotide in the nucleic acid sequence.

In one embodiment of any aspect, the nucleic acid sequence excludes the entire BCL11A enhancer functional regions and excludes the entire BCL11A coding region. In one embodiment of any aspect,

, the nucleic acid sequence is selected from the group consisting of SEQ ID NOS: 37, 38, 39, 40, 41, 42, 43, 44, 45, 46, 47, 48, 50, 51, 52, 53, 54, 55, 56, 57, and 62 as shown in Table 2.

In one embodiment of any aspect, the chemical modification is located at one or more terminal nucleotides in nucleic acid sequence. Exemplary chemical modifications include but are not limited to: 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag).

In one embodiment of any aspect, the chemical modification is located only at the 3′ end, or added only at the 5′ end, or added at both the 5′ and 3′ ends of the nucleic acid sequence. In one embodiment of any aspect, the chemical modification is located to first three nucleotides and to the last three nucleotides of the nucleic acid sequence.

In one embodiment of any aspect, the nucleic acid sequence further comprising a crRNA/tracrRNA sequence.

In one embodiment of any aspect, the nucleic acid sequence is a single guide RNA (sgRNA).

In one embodiment of any aspect, the nucleic acid sequence excludes the entire region between the human chromosome 2 location 60725424 to 60725688 (DHS +55 functional region), or excludes the entire region at location 60722238 to 60722466 (DHS +58 functional region), or excludes the entire region at location 60718042 to 60718186 (DHS +62 functional region), or excludes the entire region at location 60773106 to 60773435 (exon 2) of the human chromosome 2 is that according to UCSC Genome Browser hg 19 human genome assembly.

In one embodiment of any aspect, the base editor protein is a third generation base editor. In one embodiment of any aspect, the base editor protein is A3A (N57Q)-BE3, A3A-BE3, or A3A (N57G)-BE3.

In one embodiment of any aspect, the RNP complex is for use in the ex vivo targeted genome editing of at least one target in a progenitor cell selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one embodiment of any aspect, the RNP complex is for use in an ex vivo method of producing a progenitor cell or a population of progenitor cells wherein the cells or the differentiated progeny therefrom have decreased BCL11A mRNA or protein expression.

In one embodiment of any aspect, the RNP complex is for use in an ex vivo method of producing an isolated genetic engineered human cell or a population of genetic engineered human cells having at least one genetic modification.

In one embodiment of any aspect, the RNP complex is for use in an ex vivo method of increasing fetal hemoglobin levels in a cell or in a mammal.

In one embodiment of any aspect, the RNP complex is used in the electroporation of cells.

In one embodiment of any aspect, the isolated progenitor cell or isolated cell is a hematopoietic progenitor cell or a hematopoietic stem cell. In one embodiment of any aspect, the hematopoietic progenitor is a cell of the erythroid lineage. In one embodiment of any aspect, the isolated progenitor cell or isolated cell is an induced pluripotent stem cell.

In one embodiment of any aspect, the progenitor cell or human cell acquires at least one genetic modification. In one embodiment of any aspect, the at least one genetic modification is a deletion, insertion or substitution of the genetic sequence of the cell. In one embodiment of any aspect, the at least one genetic modification is any base substitution. In one embodiment of any aspect, the at least one genetic modification is a C to T, G, or A substitution.

In one embodiment of any aspect, the at least one genetic modification is located between chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) of the human chromosome 2 is that according to UCSC Genome Browser hg 19 human genome assembly.

In one embodiment of any aspect, the at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one embodiment of any aspect, the at least one genetic modification results in the increase in fetal hemoglobin induction.

In one embodiment of any aspect, the RNP complex is for use in treating beta-hemoglobin disorders selected from the group consisting of: sickle cell disease and beta-thalassemia.

Another aspect provided herein provides a ribonucleoprotein (RNP) complex comprising (a) a base editor protein; and (b) at least two nucleic acid sequences shown in Table 1, SEQ ID NOS: 1-139, wherein there is at least one chemical modification to a nucleotide in the nucleic acid sequence.

Another aspect provided herein provides a nucleoprotein (RNP) complex comprising (a) a base editor protein; and (b) at least one guide RNAs targeting an enhancer region and at least one guide RNA targeting an additional sequence.

In one embodiment of any aspect, the enhancer region is a BCL11A enhancer region.

In one embodiment of any aspect, the at least one additional sequence is selected from the group selected from: gamma-globin promoter mutant sequence associated with HPFH, sickle cell HbS mutant sequence, sickle cell HbC mutant sequence, sickle cell HbD mutant sequence, β-Thalassemia HbE mutant sequence, and alpha-globin sequences

In one embodiment of any aspect, the RNP complex is for use in correcting the β-Thalassemia HBB-28 A to G mutation.

Another aspect provided herein provides a nucleoprotein (RNP) complex comprising (a) a base editor protein; and (b) at least one guide RNAs targeting an enhancer region and at least one guide RNA targeting a promoter sequence.

In one embodiment of any aspect, the enhancer region is a BCL11A enhancer region. In one embodiment of any aspect, the enhancer region is a BCL11A enhancer region is a +55 enhancer, +58 enhancer, or +62 enhancer.

In one embodiment of any aspect, the promoter site is a HBG½ promoter site. In one embodiment of any aspect, the HBG½ promoter site is −115 or −198.

Another aspect provided herein provides a ribonucleoprotein (RNP) complex comprising: (a) A3A (N57Q)-BE3; and (b) a nucleic acid sequences having the sequence of SEQ IN: 42, wherein there is at least one chemical modification to a nucleotide in the nucleic acid sequence.

Yet another aspect provided herein provides a composition comprising any of the RNP complexes described herein.

In one embodiment of any aspect, the composition is for use in the ex vivo targeted genome editing of the BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one embodiment of any aspect, the composition is for use in an ex vivo method of producing a progenitor cell or a population of progenitor cells wherein the cells or the differentiated progeny therefrom have decreased BCL11A mRNA or protein expression.

In one embodiment of any aspect, the composition is for use in an ex vivo method of producing an isolated genetic engineered human cell or a population of progenitor cells having at least one genetic modification.

In one embodiment of any aspect, the composition is for use in an ex vivo method for increasing fetal hemoglobin levels in a cell or in a mammal.

In one embodiment of any aspect, the composition is used in the electroporation of cells. In one embodiment of any aspect, the step of electroporation is performed in a solution comprising glycerol.

Another aspect provided herein provides a method for producing a progenitor cell or a population of progenitor cells having decreased BCL11A mRNA or protein expression, the method comprising contacting an isolated progenitor cell with an effective amount of any RNP complex or composition described herein, whereby the contacted cells or the differentiated progeny cells therefrom have decreased BCL11A mRNA or protein expression.

Another aspect provided herein provides a method for producing an isolated genetic engineered human cell or a population of genetic engineered isolated human cells having at least one genetic modification, the method comprising contacting an isolated cell or a population of cells with an effective amount of any RNP complex or composition described herein, wherein the at least one genetic modification produced is located in human chromosome 2 is that according to UCSC Genome Browser hg 19 human genome assembly.

Another aspect provided herein provides a method of increasing fetal hemoglobin levels in a cell, the method comprising the steps of: contacting an isolated cell with an effective amount of a composition of any RNP complex or composition described herein, thereby causing at least one genetic modification at the human chromosome 2 is that according to UCSC Genome Browser hg 19 human genome assembly therein, whereby fetal hemoglobin expression is increased in said cell, or its progeny, relative to said cell prior to said contacting.

Another aspect provided herein provides an isolated genetic engineered human cell or a population of genetic engineered human cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2).

Another aspect provided herein provides an isolated genetic engineered human cell or a population of genetic engineered human cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

Another aspect provided herein provides a population of genetically edited progenitor cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2).

Another aspect provided herein provides a population of genetically edited progenitor cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

Another aspect provided herein provides a composition comprising any of the isolated genetic edited human cells described herein.

Another aspect provided herein provides a composition comprising any of the genetically edited progenitor cells described herein.

Another aspect provided herein provides a method for increasing fetal hemoglobin levels in a mammal in need thereof, the method comprising the steps of: (a) ex vivo contacting an isolated hematopoietic progenitor cell isolated from said mammal with an effective amount of any of the RNP complexes or composition described herein, whereby the base editor protein targets the genomic DNA of the cell, causing at least one genetic modification therein, whereby fetal hemoglobin expression is increased in said cell or the differentiated progeny cells therefrom, relative to expression prior to said contacting; and (b) transplanting the contacted cells of (a) or culture expanded cells therefrom into said mammal.

Another aspect provided herein provides a method for increasing fetal hemoglobin levels in a mammal in need thereof, the method comprising transplanting into the mammal: (a) any of the isolated genetic engineered human cells described herein, or (b) any of the populations of genetically edited progenitor cells described herein, or (c) any of the compositions described herein, or (d) or the progeny cells from of (a) or (b).

### Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

As used herein, the phrase “agent that binds the genomic DNA of the cell on chromosome 2 location 60725424 to 60725688 (+55 functional region), at location 60722238 to 60722466 (+58 functional region), and/or at location 60718042 to 60718186 (+62 functional region)” refers to small molecules, nucleic acids, proteins, peptides or oligonucleotides that can bind to the location within the genomic DNA (e.g., chromosome 2 location 60725424 to 60725688 (+55 functional region), at location 60722238 to 60722466 (+58 functional region), and/or at location 60718042 to 60718186 (+62 functional region)) and represses mRNA or protein expression of BCL11A in a cell by at least 20% compared to the mRNA or protein level of BCL11A in a cell not treated with such an agent. In one embodiment, the agent “interferes with BCL11A interactions with BCL11A binding partners,” as that phrase is used herein.

As used herein, “chromosome 2 location 60725424 to 60725688 (+55 functional region)” refers to a region of a chromosome comprising, consisting of, or consisting essentially of a nucleic acid sequence of SEQ ID NO: 234.

As used herein, “chromosome 2 location 60722238 to 60722466 (+58 functional region)” refers to a region of a chromosome comprising, consisting of, or consisting essentially of a nucleic acid sequence of SEQ ID NO: 235.

As used herein, “chromosome 2 location 60718042 to 60718186 (+62 functional region)” refers to a region of a chromosome comprising, consisting of, or consisting essentially of a nucleic acid sequence of SEQ ID NO: 236.

As used herein, “BCL11A exon 2” refers to a region of a chromosome comprising, consisting of, or consisting essentially of a nucleic acid sequence of SEQ ID NO: 237.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A “nucleic acid”, as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

By “interferes with BCL11A interactions with BCL11A binding partners” is meant that the amount of interaction of BCL11A with the BCL11A binding partner is at least 5% lower in populations treated with a BCL11A inhibitor, than a comparable, control population, wherein no BCL11A inhibitor is present. It is preferred that the amount of interaction of BCL11A with the BCL11A binding partner in a BCL11A-inhibitor treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no BCL11A inhibitor is added. At a minimum, BCL11A interaction can be assayed by determining the amount of BCL11A binding to the BCL11A binding partner using techniques standard in the art, including, but not limited to, mass spectrometry, immunoprecipitation, or gel filtration assays. Alternatively, or in addition, BCL11A activity can be assayed by measuring fetal hemoglobin expression at the mRNA or protein level following treatment with a candidate BCL11A inhibitor.

In one embodiment, BCL11A activity is the interaction of BCL11A with its binding partners: GATA-1, FOG-1, components of the NuRD complex, matrin-3, MTA2 and RBBP7. Accordingly, any antibody or fragment thereof, small molecule, chemical or compound that can block this interaction is considered an inhibitor of BCL11A activity.

As used herein, the term “genetic engineered cell” refers to a cell that comprises at least one genetic modification, as that term is used herein.

As used herein, the term “genetic modification” refers to a disruption at the genomic level resulting in a decrease in BCL11A expression or activity in a cell. Exemplary genetic modifications can include deletions, frame shift mutations, point mutations, exon removal, removal of one or more DNAse 1-hypersensitive sites (DHS) (e.g., 2, 3, 4 or more DHS regions), etc.

By “inhibits BCL11A expression” is meant that the amount of expression of BCL11A is at least 5% lower in a cell or cell population treated with an RNP complex described herein, than a comparable, control cell or cell population, wherein no RNP complex is present. It is preferred that the percentage of BCL11A expression in a treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no RNP complex is added.

By “inhibits BCL11A activity” is meant that the amount of functional activity of BCL11A is at least 5% lower in a cell or cell population treated with the methods described herein, than a comparable, control cell or population, wherein no an RNP complex is present. It is preferred that the percentage of BCL11A activity in a BCL11A-inhibitor treated population is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more than a comparable control treated population in which no an RNP complex is added. At a minimum, BCL11A activity can be assayed by determining the amount of BCL11A expression at the protein or mRNA levels, using techniques standard in the art. Alternatively, or in addition, BCL11A activity can be determined using a reporter construct, wherein the reporter construct is sensitive to BCL11A activity. The γ-globin locus sequence is recognizable by the nucleic acid-binding motif of the BCL11A construct.

As used herein the term “cleaves” generally refers to the generation of a double-stranded break in the DNA genome at a desired location.

As used herein, the terms “adenine”, “guanine”, “cytosine”, “thymine”, “uracir” and “hypoxanthine” (the nucleobase in inosine) refer to nucleobases.

As used herein, the terms “adenosine”, “guanosine”, “cytidine”, “thymidine”, “uridine” and “inosine”, refer to the nucleobases linked to the (deoxy)ribosyl sugar.

As used herein, the term “effective amount of a composition comprising at least an RNP complex” refers to an amount of RNP complex that yields sufficient base editing activity to generate a base substitution without a double-stranded break in the desired location of the genome. In one embodiment, the effective amount of an RNP complex includes a base substitution at the desired genetic locus in at least 20% of the cells in a population contacted with the composition (e.g., at least 30%, at least 40%, at least 50%, at least 60%, at least 70%, at least 80%, at least 90%, at least 95%, at least 99%, or even 100% of the cells in the population comprise a genetic modification produced by the RNP complex or composition thereof).

As used herein the term “increasing the fetal hemoglobin levels” in a cell indicates that fetal hemoglobin is at least 5% higher in populations treated with an agent that disrupts BCL11A mRNA or protein expression (e.g., n RNP complex) by binding to genomic DNA at chromosome 2 location 60,716,189-60,728,612, than in a comparable, control population, wherein no agent is present. It is preferred that the percentage of fetal hemoglobin expression in a population treated with such an agent that binds the genomic DNA at chromosome 2 location 60,716,189-60,728,612 is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a control treated population of comparable size and culture conditions. The term “control treated population” is used herein to describe a population of cells that has been treated with identical media, viral induction, nucleic acid sequences, temperature, confluence, flask size, pH, etc., with the exception of the addition of the agent that binds genomic DNA at chromosome 2 location 60,716,189-60,728,612. In one embodiment, any method known in the art can be used to measure an increase in fetal hemoglobin expression, e. g. Western Blot analysis of fetal γ-globin protein and quantifying mRNA of fetal γ-globin.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of human hematopoietic progenitor cells, e.g., a substantially pure population of human hematopoietic progenitor cells as compared to a heterogeneous population of cells comprising human hematopoietic progenitor cells and cells from which the human hematopoietic progenitor cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not hematopoietic progenitor cells as defined by the terms herein.

A “subject,” as used herein, includes any animal that exhibits a symptom of a monogenic disease, disorder, or condition that can be treated with the cell-based therapeutics, and methods disclosed elsewhere herein. In preferred embodiments, a subject includes any animal that exhibits symptoms of a disease, disorder, or condition of the hematopoietic system, e.g., a hemoglobinopathy, that can be treated with the gene therapy/cell-based therapeutics, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (lower or higher amounts than a “normal” or “healthy” subject) of one or more physiological activities that can be modulated by gene therapy.

In one embodiment, as used herein, “prevent,” and similar words such as “prevented,” “preventing” etc., indicate an approach for preventing, inhibiting, or reducing the likelihood of the occurrence or recurrence of, a disease or condition. In another embodiment, the term refers to delaying the onset or recurrence of a disease or condition or delaying the occurrence or recurrence of the symptoms of a disease or condition. In another embodiment, as used herein, “prevention” and similar words includes reducing the intensity, effect, symptoms and/or burden of a disease or condition prior to onset or recurrence of the disease or condition.

As used herein, the term “treating” includes reducing or alleviating at least one adverse effect or symptom of a condition, disease or disorder. For example, the term “treating” and “treatment” refers to administering to a subject an effective amount of a composition, e.g., an effective amount of a composition comprising a population of hematopoietic progenitor cells so that the subject has a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, disease stabilization (e.g., not worsening), delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment can improve the disease condition, but may not be a complete cure for the disease. In some embodiments, treatment can include prophylaxis. However, in alternative embodiments, treatment does not include prophylaxis.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

As used herein, “prevention” or “preventing,” when used in reference to a disease, disorder or symptoms thereof, refers to a reduction in the likelihood that an individual will develop a disease or disorder, e.g., a hemoglobinopathy. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

In connection with contacting a cell with an RNP complex or composition thereof to decrease BCL11A expression, the phrase “increasing fetal hemoglobin levels in a cell” indicates that fetal hemoglobin in a cell or population of cells is at least 5% higher in the cell or population of cells treated with the RNP complex or composition thereof, than a comparable, control population, wherein no RNP complex is present. It is preferred that the fetal hemoglobin expression in a RNP complex treated cell is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a comparable control treated population. The term “control treated population” is used herein to describe a population of cells that has been treated with identical media, viral induction, nucleic acid sequences, temperature, confluence, flask size, pH, etc., with the exception of the addition of the BCL11A inhibitor.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears. In some preferred embodiments, a mammal is a human.

Accordingly, in one embodiment, the mammal has been diagnosed with a hemoglobinopathy. In a further embodiment, the hemoglobinopathy is a β-hemoglobinopathy. In one preferred embodiment, the hemoglobinopathy is a sickle cell disease. As used herein, “sickle cell disease” can be sickle cell anemia, sickle-hemoglobin C disease (HbSC), sickle beta-plus-thalassemia (HbS/β+), or sickle beta-zero-thalassemia (HbS/β0). In another preferred embodiment, the hemoglobinopathy is a β-thalassemia.

As used herein, the term “hemoglobinopathy” means any defect in the structure or function of any hemoglobin of an individual, and includes defects in the primary, secondary, tertiary or quaternary structure of hemoglobin caused by any mutation, such as deletion mutations or substitution mutations in the coding regions of the β-globin gene, or mutations in, or deletions of, the promoters or enhancers of such genes that cause a reduction in the amount of hemoglobin produced as compared to a normal or standard condition. The term further includes any decrease in the amount or effectiveness of hemoglobin, whether normal or abnormal, caused by external factors such as disease, chemotherapy, toxins, poisons, or the like.

In one embodiment, the term “effective amount”, as used herein, refers to the amount of a cell composition that is safe and sufficient to treat, lesson the likelihood of, or delay the development of a hemoglobinopathy. The amount can thus cure or result in amelioration of the symptoms of the hemoglobinopathy, slow the course of hemoglobinopathy disease progression, slow or inhibit a symptom of a hemoglobinopathy, slow or inhibit the establishment of secondary symptoms of a hemoglobinopathy or inhibit the development of a secondary symptom of a hemoglobinopathy. The effective amount for the treatment of the hemoglobinopathy depends on the type of hemoglobinopathy to be treated, the severity of the symptoms, the subject being treated, the age and general condition of the subject, the mode of administration and so forth. Thus, it is not possible or prudent to specify an exact “effective amount”. However, for any given case, an appropriate “effective amount” can be determined by one of ordinary skill in the art using only routine experimentation.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### BRIEF DESCRIPTION OF DRAWINGS

FIG. 1a-1f show base editing the +58 BCL11A erythroid enhancer in human CD34+ HSPCs. (FIG. 1a), Five sgRNAs targeting the core +58 BCL11A erythroid enhancer TGN₇₋₉WGATAR half E-box/GATA binding motif (shown in box) with predominant base editing position indicated by arrowhead and PAM shaded. (FIG. 1b), Base editing by A3A (N57Q)-BE3 complexed with five sgRNAs in healthy donor human CD34+ HSPCs by deep sequence analysis. (FIG. 1c) HbF levels by HPLC analysis. Block dots represent three healthy donors. (FIG. 1d) HbF levels following RNP dose-response, with three colors representing three healthy donors. (FIG. 1e) Dose-dependent C base editing by A3A (N57Q)-BE3 complexed with sgRNA-1620 by deep sequence analysis. (FIG. 10 Targeting same core +58 BCL11A erythroid enhancer TGN7-9WGATAR half E-box/GATA binding motif (shown in box) by A3A (N57Q)-BE3:sgRNA-1620 and 3xNLS-SpCas9:sgRNA-1617 with predominant base editing or cleavage position respectively indicated by arrowhead and PAM shaded. HbF levels in erythroid colonies, organized by genotype.

FIG. 2a-2d show therapeutic base editing in SCD patient CD34+ HSPCs. (FIG. 2a) Base editing in two plerixafor mobilized SCD CD34+ HSPC donors by A3A (N57Q)-BE3:sgRNA-1620 by deep sequence analysis. (FIG. 2b) HbF induction by HPLC analysis. (FIG. 2c) Phase-contrast microscope representative image of enucleated erythroid progeny 30 minutes after MBS treatment from unedited and A3A (N57Q)-BE3:sgRNA-1620 base edited SCD #2 CD34+ HSPCs. Red arrows indicate sickle forms. (FIG. 2d) Quantification of sickle forms from unedited and base edited enucleated erythroid cells at 30 minutes following MBS treatment.

FIG. 3a-3f show therapeutic and multiplex base editing in β-thalassemia patient CD34+ HSPCs. (FIG. 3a) Base editing in P-thalassemia donors β⁰β⁺_{#1} and β⁰β^{E} CD34+ HSPCs by A3A (N57Q)-BE3:sgRNA-1620. (FIG. 3b) 13-like globin expression by RT-qPCR normalized by a-globin. (FIG. 3c) Hemoglobin levels by HPLC analysis. Each dot indicates a technical replicate. (FIG. 3d) Base editing by A3A(N57Q)-BE3 at BCL11A+58 enhancer with sgRNA-1620 (top two rows) and HBB promoter by sgRNA-HBB-28 (bottom two rows) following single or multiplex editing in β-thalassemia donor β⁰β⁺_{#2} with HBB-28A>Gβ⁺ mutation. At the HBB-28A>G mutation position (noted with asterisk, on opposite strand to spacer), alleles are divided into corrective C>T edits and alternative C>G/A edits. (FIG. 3e) β-like globin expression by RT-qPCR normalized by a-globin. (FIG. 3f) Hemoglobin levels by HPLC analysis. Each dot indicates a biological replicate.

FIG. 4a-4g show efficient C>T base editing in HSCs. (FIG. 4a) Base editing at sgRNA-1620 C6 position in sorted immunophenotypically enriched HSC (CD34+ CD38− CD90+ CD45RA−) or HPC (CD34+ CD38+) populations by deep sequence analysis. (FIG. 4b) Base editing at C6 position in Pyronin Y and Hoechst stained and sorted G0, G1, S and G2/M phase CD34+ HSPCs. (FIG. 4c) Base editing at C6 position in engrafting HSPCs, B cells, erythroid and unfractionated bone marrow as compared with input HSPCs following 1 or 2 rounds of electroporation (1 EP, 2 EP). (FIG. 4d) Human bone marrow chimerism 16 weeks following infusion with CD34+ HSPCs electroporated once or twice with A3A(N57Q)-BE3:sgRNA-1620. (FIG. 4e) Base editing at C6 position in bone marrow 16 weeks after secondary transplantation by Sanger sequence analysis. (FIG. 4f) HbF induction in engrafted bone marrow erythroid cells. (FIG. 4g) Correlation of base editing frequency and HbF level. Spearman correlation coefficient (r) is shown.

FIG. 5a-5b show purification of A3A (N57Q)-BE3 protein. (FIG. 5a) Purification strategy and profiles. A3A (N57Q)-BE3 protein was purified using nickel affinity, mono S cation exchange, and size exclusion columns. Desired fractions from 100% imidazole elution (nickel column tube 20-24), salt gradient elution (tube 25-35), and size exclusion (tube 11-15) were collected respectively. (FIG. 5b) Protein purity validation. Protein purity was determined using SDS-PAGE and gel staining. Total clear protein lysate and ion exchange (IEX) samples and fractions were loaded on SDS-PAGE gel and stained with GelCode Blue to check the purity. After immobilized metal affinity chromatography and IEX, the protein purity was estimated to be more than 99%.

FIG. 6a-6b show base editing the +58 BCL11A erythroid enhancer in human healthy donor CD34+ HSPCs. (FIG. 6a) Heatmaps showing the base edit frequencies following A3A (N57Q)-BE3:sgRNA-1617, sgRNA-1619 and sgRNA-1620 editing by deep sequence analysis. (FIG. 6b) Correlation of base edit frequencies and HbF levels following dose-response A3A (N57Q)-BE3:sgRNA-1620 editing. The Spearman correlation coefficient (r) is shown.

FIG. 7a-7i show therapeutic and multiplex base editing in β-thalassemia patient CD34+ HSPCs. (FIGS. 7a, 7d) Enucleation of in vitro differentiated erythroid cells. (FIGS. 7b, 7e) Cell size by relative forward scatter intensity of enucleated erythroid cells, normalized to healthy donor. (FIGS. 7c, 7f) Circularity of enucleated erythroid cells by imaging flow cytometry. (FIG. 7g) Heatmaps showing the base edit frequencies of BCL11A enhancer (top two panels) and HBB promoter (bottom two panels) following single and multiplex editing. (FIG. 7h) Left, Distribution of erythroid colonies by editing of BCL11A enhancer and HBB promoter following A3A (N57Q)-BE3:sgRNA-1620+sgRNA-HBB-28 multiplex base editing. Right, The number and fraction of colonies based on alleles at BCL11A enhancer and HBB promoter. (FIG. 7i) Hemoglobin measured by HPLC for A3A (N57Q)-BE3:sgRNA-1620+sgRNA-HBB-28 multiplex base editing by number of edited loci (left) or by combination of alleles (right).

FIG. 8a-8p show efficient base editing following HSPC xenotransplantation. (FIG. 8a) Viability of CD34+ HSPCs immediately prior to transplantation with 1 or 2 cycles of A3A(N57Q)-BE3:sgRNA-1620 electroporation (EP) relative to mock. (FIGS. 8b-8c) BCL11A expression by RT-qPCR in engrafted human B cells (FIG. 8b) and erythroid cells (FIG. 8c). (FIGS. 8d-8h) Percentage of engrafted human B cells (FIG. 8d), granulocytes (FIG. 8e) monocytes (FIG. 80 erythroid cells (FIG. 8g) and HSPCs (FIG. 8h) from mouse BM 16 weeks after primary transplantation. (FIGS. 8i-8m) Correlation of overall human chimerism to individual lineages after primary transplantation. The Spearman correlation coefficients (r) are shown. (FIG. 8n) Overall frequencies of C base edits in input CD34+ HSPCs and engrafted HSPCs, B cells, erythroid and BM by deep sequence analysis. (FIG. 8o) Human bone marrow chimerism 16 weeks after secondary transplantation. Donor 4 and donor 5 with 1 cycle EP and 2 cycles EP, and donor 6 with 2 cycles EP are shown. (FIG. 8p) Percentage of engrafted human B cells, myeloid cells and CD19− CD33− cells 16 weeks after secondary transplantation from donors 4, donor 5 and donor 6.

FIG. 9a-9i show representative xenografted bone marrow flow cytometry analysis. (FIGS. 9a-9d) Live cells from engrafted mouse BM. (FIG. 9e) Human cells gated from hCD45+ population, mouse cells gated from mCD45+ population. (FIG. 9f) B cells gated from hCD45+CD19+ population. (FIG. 9g) Granulocytes gated from hCD45+CD19-CD33dim with SSC high population. Monocytes gated from hCD45+CD19-CD33+ with SSC low population. (FIG. 9h) CD34+Lin-(HSPCs) gated from hCD45+CD19-CD33-CD34+ population. T cells gated from hCD45+CD19-CD33-CD3+ population. (FIG. 9i) Erythroid cells gated from hCD45-mCD45-hCD235a+ population.

FIG. 10a-10e show multiplex base editing in human CD34+ HSPCs. (FIG. 10a) Base editing (A>G) in multiplex edited bulk CD34+ HSPCs at each site within the editing window. (FIG. 10b) HbF level by HPLC in in vitro differentiated erythroid cells (bulk). (FIG. 10c) HbF levels from erythroid colonies derived from single CD34+ HSPCs. (FIG. 10d) Association of HbF induction with number of base edits at all four targeting sites (+58 and +55 BCL11A enhancer and HBG½ promoter −115 and −198). (FIG. 10e) Association of HbF levels with the sites edited. Targeting more sites led to greater HbF induction. For BCL11A target sites, biallelic editing was associated with potent HbF induction.

### DETAILED DESCRIPTION

### Hemoglobinopathies

Fetal hemoglobin (HbF) is a tetramer of two adult α-globin polypeptides and two fetal β-like γ-globin polypeptides. During gestation, the duplicated γ-globin genes constitute the predominant genes transcribed from the β-globin locus. Following birth, γ-globin becomes progressively replaced by adult β-globin, a process referred to as the “fetal switch” (3). The molecular mechanisms underlying this switch have remained largely undefined and have been a subject of intense research. The developmental switch from production of predominantly fetal hemoglobin or HbF (αa2γ2) to production of adult hemoglobin or HbA (α2β2) begins at about 28 to 34 weeks of gestation and continues shortly after birth at which point HbA becomes predominant. This switch results primarily from decreased transcription of the gamma-globin genes and increased transcription of beta-globin genes. On average, the blood of a normal adult contains only about 2% HbF, though residual HbF levels have a variance of over 20 fold in healthy adults (Atweh, Semin. Hematol. 38(4):367-73 (2001)).

Hemoglobinopathies encompass a number of anemias of genetic origin in which there is a decreased production and/or increased destruction (hemolysis) of red blood cells (RBCs). These disorders also include genetic defects that result in the production of abnormal hemoglobins with a concomitant impaired ability to maintain oxygen concentration. Some such disorders involve the failure to produce normal β-globin in sufficient amounts, while others involve the failure to produce normal β-globin entirely. These disorders specifically associated with the β-globin protein are referred to generally as β-hemoglobinopathies. For example, β-thalassemias result from a partial or complete defect in the expression of the β-globin gene, leading to deficient or absent HbA. Sickle cell anemia results from a point mutation in the β-globin structural gene, leading to the production of an abnormal (sickled) hemoglobin (HbS). HbS RBCs are more fragile than normal RBCs and undergo hemolysis more readily, leading eventually to anemia (Atweh, Semin. Hematol. 38(4):367-73 (2001)). Moreover, the presence of a BCL11A genetic variant, HBS1L-MYB variation, ameliorates the clinical severity in beta-thalassemia. This variant has been shown to be associated with HbF levels. It has been shown that there is an odds ratio of 5 for having a less severe form of beta-thalassemia with the high-HbF variant (Galanello S. et al., 2009, Blood, in press).

The search for treatment aimed at reduction of globin chain imbalance in patients with β-hemoglobinopathies has focused on the pharmacologic manipulation of fetal hemoglobin (α2γ2; HbF). The important therapeutic potential of such approaches is indicated by observations of the mild phenotype of individuals with co-inheritance of both homozygous β-thalassemia and hereditary persistence of fetal hemoglobin (HPFH), as well as by those patients with homozygous β-thalassemia who synthesize no adult hemoglobin, but in whom a reduced requirement for transfusions is observed in the presence of increased concentrations of fetal hemoglobin. Furthermore, it has been observed that certain populations of adult patients with 13 chain abnormalities have higher than normal levels of fetal hemoglobin (HbF), and have been observed to have a milder clinical course of disease than patients with normal adult levels of HbF. For example, a group of Saudi Arabian sickle-cell anemia patients who express 20-30% HbF have only mild clinical manifestations of the disease (Pembrey, et al., Br. J. Haematol. 40: 415-429 (1978)). It is now accepted that β-hemoglobinopathies, such as sickle cell anemia and the β-thalassemias, are ameliorated by increased HbF production. (Reviewed in Jane and Cunningham Br. J. Haematol. 102: 415-422 (1998) and Bunn, N. Engl. J. Med. 328: 129-131 (1993)).

While the molecular mechanisms controlling the in vivo developmental switch from γ- to β-globin gene expression are currently unknown, there is accumulating evidence that external factors can influence γ-globin gene expression. The first group of compounds discovered having HbF reactivation activity were cytotoxic drugs. The ability to cause de novo synthesis of HbF by pharmacological manipulation was first shown using 5-azacytidine in experimental animals (DeSimone, Proc Natl Acad Sci U S A. 79(14):4428-31 (1982)). Subsequent studies confirmed the ability of 5-azacytidine to increase HbF in patients with β-thalassemia and sickle cell disease (Ley, et al., N. Engl. J. Medicine, 307: 1469-1475 (1982), and Ley, et al., Blood 62: 370-380 (1983)). Additional experiments demonstrated that baboons treated with cytotoxic doses of arabinosylcytosine (ara-C) responded with striking elevations of F-reticulocytes (Papayannopoulou et al., Science. 224(4649):617-9 (1984)), and that treatment with hydroxyurea led to induction of γ-globin in monkeys or baboons (Letvin et. al., N Engl J Med. 310(14):869-73 (1984)).

The second group of compounds investigated for the ability to cause HbF reactivation activity was short chain fatty acids. The initial observation in fetal cord blood progenitor cells led to the discovery that γ-aminobutyric acid can act as a fetal hemoglobin inducer (Perrine et al., Biochem Biophys Res Commun.148(2):694-700 (1987)). Subsequent studies showed that butyrate stimulated globin production in adult baboons (Constantoulakis et al., Blood. Dec; 72(6):1961-7 (1988)), and it induced γ-globin in erythroid progenitors in adult animals or patients with sickle cell anemia (Perrine et al., Blood. 74(1):454-9 (1989)). Derivatives of short chain fatty acids such as phenylbutyrate (Dover et al., Br J Haematol. 88(3):555-61 (1994)) and valproic acid (Liakopoulou et al., 1: Blood. 186(8):3227-35 (1995)) also have been shown to induce HbF in vivo. Given the large number of short chain fatty acid analogs or derivatives of this family, there are a number of potential compounds of this family more potent than butyrate. Phenylacetic and phenylalkyl acids (Torkelson et al., Blood Cells Mol Dis. 22(2):150-8. (1996)), which were discovered during subsequent studies, were considered potential HbF inducers as they belonged to this family of compounds. Presently, however, the use of butyrate or its analogs in sickle cell anemia and β-thalassemia remains experimental and cannot be recommended for treatment outside of clinical trials.

Clinical trials aimed at reactivation of fetal hemoglobin synthesis in sickle cell anemia and β-thalassemia have included short term and long term administration of such compounds as 5-azacytidine, hydroxyurea, recombinant human erythropoietin, and butyric acid analogs, as well as combinations of these agents. Following these studies, hydroxyurea was used for induction of HbF in humans and later became the first and only drug approved by the Food and Drug Administration (FDA) for the treatment of hemoglobinopathies. However, varying drawbacks have contraindicated the long term use of such agents or therapies, including unwanted side effects and variability in patient responses. For example, while hydroxyurea stimulates HbF production and has been shown to clinically reduce sickling crisis, it is potentially limited by myelotoxicity and the risk of carcinogenesis. Potential long term carcinogenicity would also exist in 5-azacytidine-based therapies. Erythropoietin-based therapies have not proved consistent among a range of patient populations. The short half-lives of butyric acid in vivo have been viewed as a potential obstacle in adapting these compounds for use in therapeutic interventions. Furthermore, very high dosages of butyric acid are necessary for inducing γ-globin gene expression, requiring catheterization for continuous infusion of the compound. Moreover, these high dosages of butyric acid can be associated with neurotoxicity and multiorgan damage (Blau, et al., Blood 81: 529-537 (1993)). While even minimal increases in HbF levels are helpful in sickle cell disease, β-thalassemias require a much higher increase that is not reliably, or safely, achieved by any of the currently used agents (Olivieri, Seminars in Hematology 33: 24-42 (1996)).

Identifying natural regulators of HbF induction and production could provide a means to devise therapeutic interventions that overcome the various drawbacks of the compounds described above. Recent genome-wide association studies have yielded insights into the genetic basis of numerous complex diseases and traits (McCarthy et al., Nat Rev Genet 9, 356 (2008) and Manolio et. al. J Clin Invest 118, 1590 (2008)). However, in the vast majority of instances, the functional link between a genetic association and the underlying pathophysiology remains to be uncovered. The level of fetal hemoglobin (HbF) is inherited as a quantitative trait and clinically important, given its above-mentioned and well-characterized role in ameliorating the severity of the principal β-hemoglobinopathies, sickle cell disease and β-thalassemia (Nathan et. al., Nathan and Oski's hematology of infancy and childhood ed. 6th, pp. 2 v. (xiv, 1864, xli p.) 2003)). Two genome-wide association studies have identified three major loci containing a set of five common single nucleotide polymorphisms (SNPs) that account for ˜20% of the variation in HbF levels (Lettre et al., Proc Natl Acad Sci U S A (2008); Uda et al., Proc Natl Acad Sci U S A 105, 1620 (2008); Menzel et al., Nat Genet 39, 1197 (2007)). Moreover, several of these variants appear to predict the clinical severity of sickle cell disease (Lettre et al., Proc Natl Acad Sci U S A (2008)) and at least one of these SNPs may also affect clinical outcome in β-thalassemia (Uda et al., Proc Natl Acad Sci U S A 105, 1620 (2008)). The SNP with the largest effect size, explaining over 10% of the variation in HbF, is located in the second intron of a gene on chromosome 2, BCL11A. Whereas BCL11A, a C2H2-type zinc finger transcription factor, has been investigated for its role in lymphocyte development (Liu et al., Nat Immunol 4, 525 (2003) and Liu et al., Mol Cancer 5, 18 (2006)), its role in red blood cell production or globin gene regulation has not been previously assessed.

At the onset of the recombinant DNA era, studies of globin gene structure provided a strong molecular foundation for interrogating the fetal globin switch. Considerable effort has focused on delineating the cis-elements within the β-globin locus necessary for proper regulation of the genes within the β-like globin cluster. These studies relied on naturally occurring mutations and deletions that dramatically influence HbF levels in adults, and have been complemented by generation of transgenic mice harboring portions of the cluster (Nathan et. al., Nathan and Oski's hematology of infancy and childhood ed. 6th, pp. 2 v. (xiv, 1864, xli p.) 2003) and G. Stamatoyannopoulos, Exp Hematol 33, 259 (2005)). Although the precise cis-elements required for globin switching remain ill-defined, findings in transgenic mice have strongly indicated that the γ-globin genes are autonomously silenced in the adult stage, a finding that is most compatible with the absence of fetal-stage specific activators or the presence of a stage-specific repressor. The results of recent genetic association studies provide candidate genes to interrogate for their involvement in control of the γ-globin genes, such as BCL11A.

As used herein, treating or reducing a risk of developing a hemoglobinopathy in a subject means to ameliorate at least one symptom of hemoglobinopathy. In one aspect, the invention features methods of treating, e.g., reducing severity or progression of, a hemoglobinopathy in a subject. In another aspect, the methods can also be used to reduce a risk of developing a hemoglobinopathy in a subject, delaying the onset of symptoms of a hemoglobinopathy in a subject, or increasing the longevity of a subject having a hemoglobinopathy. In one aspect, the methods can include selecting a subject on the basis that they have, or are at risk of developing, a hemoglobinopathy, but do not yet have a hemoglobinopathy, or a subject with an underlying hemoglobinopathy. Selection of a subject can include detecting symptoms of a hemoglobinopathy, a blood test, genetic testing, or clinical recordings. If the results of the test(s) indicate that the subject has a hemoglobinopathy, the methods also include administering the compositions described herein, thereby treating, or reducing the risk of developing, a hemoglobinopathy in the subject. For example, a subject who is diagnosis of SCD with genotype HbSS, HbS/β0 thalassemia, HbSD, or HbSO, and/or HbF <10% by electrophoresis.

As used herein, the term “hemoglobinopathy” refers to a condition involving the presence of an abnormal hemoglobin molecule in the blood. Examples of hemoglobinopathies include, but are not limited to, SCD and THAL. Also included are hemoglobinopathies in which a combination of abnormal hemoglobins is present in the blood (e.g., sickle cell/Hb-C disease). An exemplary example of such a disease includes, but is not limited to, SCD and THAL. SCD and THAL and their symptoms are well-known in the art and are described in further detail below. Subjects can be diagnosed as having a hemoglobinopathy by a health care provider, medical caregiver, physician, nurse, family member, or acquaintance, who recognizes, appreciates, acknowledges, determines, concludes, opines, or decides that the subject has a hemoglobinopathy.

The term “SCD” is defined herein to include any symptomatic anemic condition which results from sickling of red blood cells. Manifestations of SCD include: anemia; pain; and/or organ dysfunction, such as renal failure, retinopathy, acute-chest syndrome, ischemia, priapism, and stroke. As used herein the term “SCD” refers to a variety of clinical problems attendant upon SCD, especially in those subjects who are homozygotes for the sickle cell substitution in HbS. Among the constitutional manifestations referred to herein by use of the term of SCD are delay of growth and development, an increased tendency to develop serious infections, particularly due to pneumococcus, marked impairment of splenic function, preventing effective clearance of circulating bacteria, with recurrent infarcts and eventual destruction of splenic tissue. Also included in the term “SCD” are acute episodes of musculoskeletal pain, which affect primarily the lumbar spine, abdomen, and femoral shaft, and which are similar in mechanism and in severity. In adults, such attacks commonly manifest as mild or moderate bouts of short duration every few weeks or months interspersed with agonizing attacks lasting 5 to 7 days that strike on average about once a year. Among events known to trigger such crises are acidosis, hypoxia, and dehydration, all of which potentiate intracellular polymerization of HbS (J. H. Jandl, Blood: Textbook of Hematology, 2nd Ed., Little, Brown and Company, Boston, 1996, pages 544-545).

As used herein, “THAL” refers to a hereditary disorder characterized by defective production of hemoglobin. In one embodiment, the term encompasses hereditary anemias that occur due to mutations affecting the synthesis of hemoglobins. In other embodiments, the term includes any symptomatic anemia resulting from thalassemic conditions such as severe or β-thalassemia, thalassemia major, thalassemia intermedia, α-thalassemias such as hemoglobin H disease. β-thalassemias are caused by a mutation in the β-globin chain, and can occur in a major or minor form. In the major form of β-thalassemia, children are normal at birth, but develop anemia during the first year of life. The mild form of 13 -thalassemia produces small red blood cells. Alpha-thalassemias are caused by deletion of a gene or genes from the globin chain.

By the phrase “risk of developing disease” is meant the relative probability that a subject will develop a hemoglobinopathy in the future as compared to a control subject or population (e.g., a healthy subject or population). For example, an individual carrying the genetic mutation associated with SCD, an A to T mutation of the β-globin gene, and whether the individual in heterozygous or homozygous for that mutation increases that individual's risk.

As used herein, the term “genome editing” refers to a reverse genetics method using artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point.

### RNP Complexes

The BCL11A protein is a stage specific regulator of fetal hemoglobin expression by repressing γ-globin induction. Within the BCL11A locus, there are defined functional regions within the BCL11A˜12 kb enhancer region that regulate expression of the BCL11A protein. The functional regions are location 60725424 to 60725688 (+55 functional region) (SEQ DI NO: 234), location 60722238 to 60722466 (+58 functional region) (SEQ ID NO: 235), and location 60718042 to 60718186 (+62 functional region) (SEQ ID NO: 236) on the human chromosome 2 according to UCSC Genome Browser hg 19 human genome assembly. Genome editing disruption at these regions have been shown to disrupt the expression of the BCL11A mRNA, expression of the BCL11A protein, and ultimately enriched the fetal hemoglobin (HbF) produced in the edited cells. The CRISPR/Cas9 technology using small single guide RNA (sgRNA or gRNA) sequences, introduced intracellularly by viral vectors, have been successful in targeted genomic targeting of these functional regions, and reduced BCL11A expression and increase HbF expression. However, the lentiviral delivery of Cas protein and sgRNA leaves the edited cells with viral genetic materials therein which may not be ideal for human therapy. Moreover, the levels of gene editing obtained by lentiviral delivery can be variable, and this can impede achieving therapeutically relevant level of gene editing for clinical applications in patients. The electroporation approach provides an alternative for BCL11A enhancer gene editing that give improved and high therapeutically relevant level for clinical uses.

In one aspect, provided herein are chemically modified synthetic nucleic acid molecules that target the BCL11A enhancer functional region or BCL11A exon 2 region in complex with a base editor to form a ribonucleoprotein (RNP) complex. Specifically, targeting the three BCL11A enhancer functional regions., these three +62, +58, and +55, or the exon 2 region. These RNP complexes are introduced into a cell via electroporation to edit the BCL11A genomic regions, at the BCL11A enhancer functional region or BCL11A exon 2 region, thereby causing a reduction of BCL11A expression in the edited cell, for example, a CD 34+ hematopoietic progenitor cell or a hematopoietic stem cell. Upon decreased BCL11A mRNA and protein in such cells, as they differentiate into mature red blood cells, there would be an increase in erythroid γ-globin levels in these cells compared to cells that had not undergone targeting editing at the BCL11A locus. These resultant cells with increased erythroid γ-globin levels can be used to treatment a hemoglobinopathy in a mammal, such as 13 -thalassemia and sickle cell anemia. In fact, edited BCL11A CD 34+ hematopoietic progenitor cell or a hematopoietic stem cell can be transplanted into individuals with a hemoglobinopathy as a cell-based gene therapy treatment for the hemoglobinopathy.

Also in other aspects, provided herein compositions comprising the RNP complexes, and methods for increasing fetal hemoglobin levels in a cell by disrupting BCL11A expression at the genomic level. Also provided herein are methods and compositions relating to the treatment of hemoglobinopathies by reinduction of fetal hemoglobin levels.

Accordingly, one aspect described herein provides a RNP complex comprising: (a) a base editor protein; and (b) a nucleic acid sequence shown in Table 1, SEQ ID NOS: 1-139, wherein there is at least one chemical modification to a nucleotide in the nucleic acid sequence.

Another aspect provided herein provides a RNP complex comprising (a) a base editor protein; and (b) at least two nucleic acid sequences shown in Table 1, SEQ ID NOS: 1-139, wherein there is at least one chemical modification to a nucleotide in the nucleic acid sequence. In one embodiment, the RNP complex has at least 3, 4, 5, 6, 7, 8, 9, 10, 11, 12, 13, 14, 15, or more nucleic acid sequences shown in Table 1. In an alternate embodiment, the RNP complex comprises at least 2, 3, 4, 5, 6, 7, 8, 9, 10, 11, 12, 13, 14, 15, or more nucleic acid sequences that are not shown in Table 1 but a nucleic acid sequence that is useful in genome editing described herein.

Another aspect provided herein provides a RNP complex comprising (a) a base editor protein; and (b) at least one guide RNAs targeting an enhancer region and at least one guide RNA targeting an additional sequence.

Another aspect provided herein provides a RNP complex comprising (a) a base editor protein; and (b) at least one guide RNAs targeting an enhancer region and at least one guide RNA targeting a promoter.

In one embodiment, the promoter is the HBG½ promoter. For example, at the HBG promoter 1 (SEQ ID NO: 238), or the HBG promoter 2 (SEQ ID NO: 239).

Another aspect provided herein provides a RNP complex comprising (a) a base editor protein; and (b) at least two guide RNAs targeting an enhancer region and at least one guide RNA targeting a promoter.

Another aspect provided herein provides a RNP complex comprising (a) a base editor protein; and (b) at least one guide RNAs targeting an enhancer region and at least two guide RNA targeting a promoter.

Another aspect provided herein provides a RNP complex comprising (a) a base editor protein; and (b) at least two guide RNAs targeting an enhancer region and at least two guide RNA targeting a promoter.

In one embodiment, the enhancer region is a BCL11A enhancer region Enhancer regions can be identified using techniques known in the art, e.g., identifying enhancer hallmarks in the chromatin.

In one embodiment, the at least one additional sequence is selected from the group selected from: gamma-globin promoter mutant sequence associated with HPFH, sickle cell HbS mutant sequence, sickle cell HbC mutant sequence, sickle cell HbD mutant sequence, β-Thalassemia HbE mutant sequence, and alpha-globin sequences. As used herein, a “mutant” sequence refers to a sequence that varies from the wild-type sequence. A mutant sequence can have a mutation that results in the onset of a disease (i.e., a disease gene. It is contemplated herein that the genome editing described herein can correct the mutation and reduce or cure the disease state.

In one embodiment, the RNP complex is for use in correcting the β-Thalassemia HBB-28 A to G mutation. One skilled in the art can determine if the A to G mutation has been corrected, e.g., genome sequencing prior to and after genome editing using methods described herein.

Another aspect provided herein provides a ribonucleoprotein (RNP) complex comprising: (a) A3A (N57Q)-BE3; and (b) a nucleic acid sequences having the sequence of SEQ IN: 42, wherein there is at least one chemical modification to a nucleotide in the nucleic acid sequence.

As described herein, a “base editor” refers to a genome editing system that induces a base substitution, e.g., an A to a T, or a G to a C, but does not induce double stranded breaks. As described herein, Cas nucleases, or other endonucleases with CRISPR genome editing are not base editors.

RNA editing is a co- or post-transcriptional process that alters transcript sequences without any change in the encoding DNA sequence. Although various types of RNA editing have been observed in single cell organisms to mammals, base modifications by deamination of adenine to inosine (A>I), or cytidine to uracil (C>U) are the major types of RNA editing in higher eukaryotes. I and U are read as guanosine (G) and thymine (T) respectively by the cellular machinery during mRNA translation and reverse transcription. In contrast to gene-editing nucleases, base editors do not introduce double-strand breaks or exogenous donor DNA templates, and induce lower levels of unwanted variable-length insertion/deletion mutations (indels). In one embodiment, the base editor is any known base editor, e.g., a third generation base editor, A3A (N57Q)-BE3, A3A-BE3, or A3A (N57G)-BE3, or any yet to be discovered based editor. Base editors are further described in, e.g., international application PCT/EP2017/065467; and U.S. patent applications US10/934,090 and US15/564,984, which are incorporated herein by reference in their entireties.

In one embodiment, the base editor is an A>I base editor. In one embodiment, the base editor is an C>U or C>T base editor. RNA-dependent ADAR1, ADAR2 and ADAR3 adenosine deaminases, and APOBEC1 cytidine deaminase are non-limiting examples of RNA base editors known in mammals. RNA sequencing studies suggest that A>I RNA base editing affects hundreds of thousands of sites, though most of A>I RNA edits occur at a low level and in non-coding intronic and untranslated regions, especially in the context of specific sequences such as Alu elements. A>I editing of protein-coding RNA sequences at a high level (>20%) is rare and thought to occur predominantly in the brain.

Unlike A>I editing catalyzed by adenosine deaminases, the prevalence and level of C>U RNA editing in different types of cells, and its enzymatic basis and regulation are poorly understood. Exemplary C>U base editors include, but are not limited to, activation-induced deaminase (AID), apolipoprotein B editing catalytic polypeptide-like (APOBEC) family, and cytidine deaminase (CDA). In one embodiment, the C>U base editor is any proteins harboring the cytidine deaminase motif for hydrolytic deamination of C to U. CDA is involved in the pyrimidine salvaging pathway. While AID causes C>U deamination of DNA, multiple studies have failed to identify any RNA editing activity for this protein. The base editor of this invention can be selected from any of the 10 APOBEC genes (APOBEC1, 2, 3A-D, 3F-H and 4) identified in humans. APOBEC3 proteins can deaminate cytidines in single-stranded (ss) DNA, and although the APOBEC proteins bind RNA, C>U deamination of RNA is known for only APOBEC1, with apolipoprotein B (APOB) mRNA as its physiological target. C>U RNA editing alters hundreds of cytidines in chloroplasts and mitochondria of flowering plants.

Mutations in the cytidine deaminase enzyme can shorten the length of the editing window and thereby partially address off target editing. In one embodiment, the base editor is mutated to reduce the length of editing window (e.g., the region along the gene sequence which the base editor can target). In one embodiment, the editing window is reduced by at least 1, 2, 3, 4, 5, 6, 7, 8, 9, 10, 11, 12, 13, 14, 15, 16, 17, 18, 19, 20, 21, 22, 23, 24, 25, or more base pairs as compared to the editing window of a wild-type base editor.

As shown in Gehrke, J M, et al. Nature Biotech. 30 July 2018, the contents of which is incorporated herein by reference in its entirety, base editors can be mutated to induce greater precision within the editing window. In one embodiment, the base editor is mutated to increases its precision and efficiency of gene editing within the editing window. In one embodiment, the base editor's precision and/or efficiency is increased at least 5%, 10%, 15%, 20%, 25%, 30%, 35%, 40%, 45%, 50%, 55%, 60%, 65%, 70%, 75%, 80%, 85%, 90%, 95%, 99%, or more as compared to a wild-type base editor. One skilled in the art can assess the precise and/or efficiency of a base editor by performing, e.g., genome sequencing of a cell targeted by a base editor and compare it to the sequence of the cell prior to editing by the base editor.

In one embodiment, the natural diversity of cytidine deaminases is leveraged to identify one with greater sequence specificity than the rat APOBEC1 (rAPO1) deaminase present in the widely used BE3 architecture. BE3 consists of a Streptococcus pyogenes Cas9 nuclease bearing a mutation that converts it into a nickase (nCas9) fused to rAPO1 and a uracil glycosylase inhibitor (UGI). In one embodiment, rAPOl is replaced in BE3 with the human APOBEC3A (A3A) cytidine deaminase to create A3A-BE3.

Another method described herein is a composition comprising any of the RNP complexes described herein. As used herein, RNP complex can refer to the RNP complex, or a composition comprising the RNP complex.

In one embodiment, the RNP complex is for use in the ex vivo targeted genome editing of the BCL11A erythroid enhancer DHS +62, +58, or +55 functional regions or the BCL11A exon 2 region in a progenitor cell purpose.

In one embodiment, the RNP complex is for use in the ex vivo targeted genome editing of at least one target in a progenitor cell selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one embodiment, the RNP complex is for use in an ex vivo method of producing a progenitor cell or a population of progenitor cells wherein the cells or the differentiated progeny therefrom have decreased BCL11A mRNA or protein expression.

In one embodiment, the RNP complex is for use in an ex vivo method of producing an isolated genetic engineered human cell or a population of genetic engineered human cells having at least one genetic modification.

In one embodiment, the RNP complex is for use in an ex vivo method of increasing fetal hemoglobin levels in a cell or in a mammal.

In one embodiment, the RNP complex is for use in treating beta-hemoglobin disorders selected from the group consisting of: sickle cell disease and beta-thalassemia.

In one embodiment of any aspect, the RNP complex is used in the electroporation of cells. Methods for electroporation are described herein below.

### Compositions and Methods for Treatment

In one embodiment of any aspect, the nucleic acid sequence excludes the entire BCL11A enhancer Accordingly, the methods and compositions provided herein are novel methods for the regulation of γ-globin expression in erythroid cells. More specifically, these activities can be harnessed in methods for the treatment of β-hemoglobinopathies by induction of γ-globin via inhibition of the BCL11A gene product.

The disclosure described herein, in one embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

In one aspect, provided herein is a modified synthetic nucleic acid molecule described herein for use in the ex vivo targeted genome editing of the BCL11A erythroid enhancer DHS +62, +58, or +55 functional regions or the BCL11A exon 2 region in a progenitor cell. In another aspect, provided herein is a modified synthetic nucleic acid molecule described herein for use in the ex vivo targeted genome editing of the HBG½ promoter, e.g., the HBG½ promoter site −115 or −198 in a progenitor cell. The purpose of the modified synthetic nucleic acid molecule described herein is to direct the base editor protein to edit the BCL11A locus at the enhancer DHS +62, +58, or +55 functional regions or the BCL11A exon 2 region.

In one embodiment, this disclosure provides a method for producing an isolated genetic engineered human cell having at least one genetic modification comprising contacting an isolated cell with an effective amount of a composition comprising a modified synthetic nucleic acid molecule and based editor described herein, whereby the DNA-targeting editor alters the genomic DNA of the cell on chromosome 2 at location 60725424 to 60725688 (+55 functional region), or at location 60722238 to 60722466 (+58 functional region), and/or at location 60718042 to 60718186 (+62 functional region), or at location 60773106 to 60773435 (exon 2) causing at least one genetic modification therein, wherein the human chromosome 2 is that according to UCSC Genome Browser hg 19 human genome assembly, of the HBG½ promoter. In one embodiment, the contacting is via electroporation.

It was found herein that electroporation of cells, as described herein, in a solution comprising glycerol increases the cell viability following electroporation, as compared to electroporation in a solution that does not comprise glycerol. Accordingly, in one embodiment, the step of electroporation is performed in a solution comprising glycerol. In one embodiment, the solution (e.g., a suitable buffer used for electroporation) comprises at least 1% glycerol. In one embodiment, the solution (e.g., a buffer used for electroporation) comprising less than 1%, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more glycerol. In one embodiment, the amount of glycerol in the solution ranges from 2-4%. In one embodiment, the amount of glycerol in the solution ranges from 1-2%, 1-3%, 1-4%, 1-5%, 1-6%, 1-7%, 1-8%, 1-9%, 1-10%, 1-20%, 1-30%, 5-10%, 5-15%, 5-20%, 5-25%, 5-30%, 10-15%, 10-20%, 10-25%, 10-30%, 15-20%, 15-25%, 15-30%, 20-25%, 20-30%, 25-30%, 2-5%, 2-6%, 2-7%, 2-8%, 2-9%, 2-10%, 3-4%, 3-5%, 3-7%, 3-8%, 3-9%, 3-10%, 4-5%, 4-6%. 4-7%. 4-8%. 4-9%, 4-10%, 5-6%, 5-7%, 5-8%, 5-9%, 6-7%, 6-8%, 6-9%, 6-10%, -8%, 7-9%, 8-9%, 8-10%, or 9-10%. Glycerol can be purified glycerol or unpurified glycerol. Glycerol can be naturally occurring or synthesized. Glycerol can be derived from various processes known in the art, e.g., from plant or animal sources in which it occurs as triglerides, or propylene. In one embodiment, the solution comprises a glycerol derivative. Glycerol derivatives are further described in, e.g., U.S. Patent Application US2008/029360, which is incorporated herein by reference in its entirety.

In one aspect, provided herein is an isolated genetic engineered human cell or a population of genetic engineered human cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) according to the methods described herein, wherein the genetic modification arise from the genomic editing made via said methods. In one embodiment, the isolated genetic engineered human cell has reduced or decreased mRNA and/or protein expression of BCL11A compared to a control cell that has no one genetic modification on chromosome 2.

In one aspect, provided herein is an isolated genetic engineered human cell or a population of genetic engineered human cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one aspect, provided herein is a population of genetically edited progenitor cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) according to the methods described herein, wherein the genetic modification arise from the genomic editing made via said methods.

In one aspect, provided herein is a population of genetically edited progenitor cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one aspect provided herein is a composition of any of the genetic engineered human cells described herein.

In one aspect provided herein is a composition of any of the population of genetically edited progenitor cells described herein.

Aspects provided herein relate to methods of increasing fetal hemoglobin levels in an isolated cell, the method comprising decreasing the BCL11A mRNA or protein expression in the cell. In one aspect, the decrease of BCL11A mRNA or protein expression is achieved by causing at least one genetic modification at the genomic DNA of the cell on chromosome 2 location 60725424 to 60725688 (+55 functional region), and/or at location 60722238 to 60722466 (+58 functional region), or at location 60718042 to 60718186 (+62 functional region), or at location 60773106 to 60773435 (exon 2) (according to UCSC Genome Browser hg 19 human genome assembly). In another aspect, the decrease of BCL11A mRNA or protein expression is achieved by causing at least one genetic modification at the genomic DNA of the cell on chromosome 2 location 60725424 to 60725688 (+55 functional region), and/or at location 60722238 to 60722466 (+58 functional region), or at location 60718042 to 60718186 (+62 functional region), or at location 60773106 to 60773435 (exon 2) that results in an epigenetic modification of the genetic function at chromosome 2.

In one aspect, provided herein is a method of increasing fetal hemoglobin levels in a cell, the method comprising the steps of: contacting an isolated cell with an effective amount of a composition comprising or any RNP complex described herein, thereby causing at least one genetic modification at the human chromosome 2 is that according to UCSC Genome Browser hg 19 human genome assembly therein, whereby fetal hemoglobin expression is increased in said cell, or its progeny, relative to said cell prior to said contacting.

In one embodiment, the at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

In one embodiment, this disclosure provides a method of increasing fetal hemoglobin levels in a cell, the method comprising the steps of: contacting an isolated cell with an effective amount of any RNP complex described herein or composition thereof, together with at least a DNA-targeting endonuclease whereby the DNA-targeting endonuclease cleaves the genomic DNA of the cell on chromosome 2 at location 60725424 to 60725688 (+55 functional region), and/or at location 60722238 to 60722466 (+58 functional region), or at location 60718042 to 60718186 (+62 functional region), or at location 60773106 to 60773435 (exon 2) causing at least one genetic modification therein, whereby fetal hemoglobin expression is increased in said cell, or its progeny, relative to said cell prior to said contacting, and wherein the human chromosome 2 is that according to UCSC Genome Browser hg 19 human genome assembly. In one embodiment, the method is an in vitro or ex vivo method. In one embodiment, the contacting is via electroporation.

As used herein, decrease in this aspect, the enhancer activity in enhancing BCL11A mRNA or protein expression in the cell is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated in any method disclosed herein.

As used herein, decrease of the BCL11A mRNA or protein expression in the cell means that protein expression is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated in any method disclosed herein.

Another aspect provided herein relates to a method of increasing fetal hemoglobin levels in an isolated cell, the method comprising providing an isolated human cell or progenitor cell and decreasing the BCL11A mRNA or protein expression in the cell.

Another aspect provided herein relates to an ex vivo or in vitro method of increasing fetal hemoglobin levels in an isolated cell, the method comprising providing an isolated human cell or progenitor cell and decreasing the BCL11A mRNA or protein expression in the cell.

Another aspect described herein relates to a use of an isolated genetic engineered human cell described herein and produced according to a method described herein for the purpose of increasing the fetal hemoglobin levels in a mammal.

Another aspect described herein relates to a use of an isolated genetic engineered human cell described herein and produced according to a method described herein for the treatment a hemoglobinopathy in a mammal.

Another aspect described herein relates to a use of an isolated genetic engineered human cell described herein and produced according to a method described herein for the manufacturer of medicament for the treatment a hemoglobinopathy in a mammal whereby the fetal hemoglobin levels in a mammal is increased.

Another aspect described herein is a composition comprising isolated genetic engineered human cells described herein and produced according to a method described herein. In one embodiment, the composition further comprises a pharmaceutically acceptable carrier.

Another aspect described herein relates to a use of a composition comprising isolated genetic engineered human cells described herein and produced according to a method described herein for the purpose of increasing the fetal hemoglobin levels in a mammal.

Another aspect described herein relates to a use of a composition comprising isolated genetic engineered human cells described herein and produced according to a method described herein for the treatment a hemoglobinopathy in a mammal.

Another aspect described herein relates to a use of a composition comprising isolated genetic engineered human cells described herein and produced according to a method described herein for the manufacturer of medicament for the treatment a hemoglobinopathy in a mammal whereby the fetal hemoglobin levels in a mammal is increased.

In one embodiment, provided herein is a use of a composition comprising isolated genetic engineered human cells for increasing the fetal hemoglobin in a mammal or for the treatment of a hemoglobinopathy in the mammal, wherein the cells have at least one epigenetic modification on chromosome 2. In one embodiment, the at least one epigenetic modification on chromosome 2 is at location 60725424 to 60725688 (+55 functional region), and/or at location 60722238 to 60722466 (+58 functional region), or at location 60718042 to 60718186 (+62 functional region), or at location 60773106 to 60773435 (exon 2) (according to UCSC Genome Browser hg 19 human genome assembly). In another embodiment, at least one epigenetic modification on chromosome 2 is made by the process of contacting the cells with an effective amount of a composition comprising a modified synthetic nucleic acid molecule described herein, together with at least a DNA-targeting enzyme whereby the DNA-targeting enzyme produces at least one epigenetic modification in the genomic DNA of the cell on chromosome 2 which affects the location 60725424 to 60725688 (+55 functional region), or at location 60722238 to 60722466 (+58 functional region), and/or at location 60718042 to 60718186 (+62 functional region), or at location 60773106 to 60773435 (exon 2) (according to UCSC Genome Browser hg 19 human genome assembly) causing therein.

In one embodiment, provided herein is a use of any isolated cells described herein or any one of the compositions described herein for the manufacture of a medicament for increasing the fetal hemoglobin in a mammal in need thereof or for the treatment of a hemoglobinopathy in a mammal.

In one embodiment, this disclosure provides a method of treatment of a hemoglobinopathy in a mammal (e.g. a human) comprising introducing a composition described herein comprising isolated genetic engineered cells having at least one genetic modification on chromosome 2, e.g., at location 60725424 to 60725688 (+55 functional region), or at location 60722238 to 60722466 (+58 functional region), and/or at location 60718042 to 60718186 (+62 functional region), or at location 60773106 to 60773435 (exon 2), or HBG½ promoter site −155 or −198, whereby fetal hemoglobin expression is increased in the mammal.

In one embodiment of this aspect and all other aspects described herein, the nucleic acid sequence of the modified synthetic nucleic acid molecule excludes the entire BCL11A enhancer functional regions.

In one embodiment of this aspect and all other aspects described herein, the nucleic acid sequence of the modified synthetic nucleic acid molecule excludes the entire BCL11A coding region.

In one embodiment of this aspect and all other aspects described herein, the nucleic acid sequence of the modified synthetic nucleic acid molecule excludes the entire BCL11A enhancer functional regions, that is excluding the entire region between the human chromosome 2 location 60725424 to 60725688 (DHS +55 functional region), or excludes the entire region at location 60722238 to 60722466 (DHS +58 functional region), or excludes the entire region at location 60718042 to 60718186 (DHS +62 functional region), or excludes the entire region at location 60773106 to 60773435 (exon 2) of the human chromosome 2 is that according to UCSC Genome Browser hg 19 human genome assembly.

In one embodiment of this aspect and all other aspects described herein, the modified synthetic nucleic acid molecule comprises a sequence selected from the group consisting of SEQ ID NOS: 1-139 or Table 1.

In one embodiment of this aspect and all other aspects described herein, the modified synthetic nucleic acid molecule comprises a sequence selected from the group consisting of SEQ ID NOS: 37, 38, 39, 40, 41, 42, 43, 44, 45, 46, 47, 48, 50, 51, 52, 53, 54, 55, 56, 57, and 62 as shown in Table 2.

In one embodiment of this aspect and all other aspects described herein, the chemical modifications on the modified synthetic nucleic acid molecule are located at one or more terminal nucleotides in the nucleic acid molecule.

In one embodiment, the chemical modification is only found on the 5′ end of the modified synthetic nucleic acid molecule. In another embodiment, the chemical modification is found only on the 3′-end. Methods of chemical modification are known in the art. MS-based modifications produces efficient BCL11A enhancer targeting guide RNAs. For example, as described in Hendel et al., 2015,

Nature Biotechnology, the reference is incorporated herein in its entirety. Chemically modified guide RNAs can purchased from Synthego.

In one embodiment of this aspect and all other aspects described herein, the chemical modification is selected from the group consisting of 2′-β-methyl 3′phosphorothioate (MS), 2′-β-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabin, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag). Chemical modifications of gRNA are further described in, e.g., Hendel A, et al. Nat Biotechnol. 2015 September; 33(9): 985-989, which is incorporated herein by reference in its entirety. Methods for generating chemical modifications on gRNA are further reviewed in, e.g., Patent Application WO2016/089433, which is incorporated herein by reference in its entirety.

Chemical modifications can be located only at the 3′ end, or added only at the 5′ end, or added at both the 5′ and 3′ ends of the modified synthetic nucleic acid molecule. In one embodiment, the chemical modification is located to first three nucleotides and to the last three nucleotides of the modified synthetic nucleic acid molecule.

In one embodiment, the nucleic acid sequence further comprising a crRNA/tracrRNA sequence. The crRNA/tracrRNA sequence is a hybrid sequence for the binding of DNA-targeting endonuclease is a Cas (CRISPR-associated) protein in forming the gene editing ribonucleoprotein (RNP) complex.

In some embodiments, the isolated progenitor cell or isolated cell is a hematopoietic progenitor cell, induced pluripotent stem cell, or hematopoietic stem cell. In some embodiments, the hematopoietic progenitor is a cell of the erythroid lineage.

In another embodiment of this aspect and all other aspects described herein, the hematopoietic cell is a cell of the erythroid lineage. Methods of isolating hematopoietic progenitor cell are well known in the art, e.g., by flow cytometric purification of CD34+ or CD133+ cells, microbeads conjugated with antibodies against CD34 or CD133, markers of hematopoietic progenitor cell. Commercial kits are also available, e.g., MACS® Technology CD34 MicroBead Kit, human, and CD34 MultiSort Kit, human, and STEMCELL™ Technology EasySep™ Mouse Hematopoietic Progenitor Cell Enrichment Kit.

In another embodiment of this aspect and all other aspects described herein, the hematopoietic stem cells, hematopoietic progenitor cells, embryonic stem cells, somatic stem cells, or progenitor cells are collected from peripheral blood, cord blood, chorionic villi, amniotic fluid, placental blood, or bone marrow.

In one embodiment of any described method, the hematopoietic progenitor or stem cells or isolated cells can be substituted with an iPSCs described herein.

In one embodiment of this aspect and all other aspects described herein, the isolated progenitor cell or isolated cell is contacted ex vivo or in vitro.

In one embodiment of this aspect, the contacted progenitor cell or contacted cell acquires at least one genetic modification. For example, the at least one genetic modification is a deletion, insertion or substitution of the nucleic acid sequence. In one embodiment, the at least one genetic modification is a deletion.

In one embodiment, the at least one genetic modification is located between chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) of the human chromosome 2 is that according to UCSC Genome Browser hg 19 human genome assembly.

In one embodiment, the method further comprises providing an isolated cell or an isolated progenitor cell or an isolated population of cells which can be progenitor cell or hematopoietic progenitor cell or an iPSCs. In one embodiment, the isolated progenitor cell is an isolated human cell. In one embodiment, the isolated human cell is a hematopoietic progenitor cell or a hematopoietic stem cell. In other embodiment, the isolated human cell is an embryonic stem cell, a somatic stem cell, a progenitor cell, or a bone marrow cell.

In one embodiment of this aspect and all other aspects described herein, the method described herein comprises contacting an embryonic stem cell, a somatic stem cell, a progenitor cell, a bone marrow cell, a hematopoietic stem cell, or a hematopoietic progenitor cell with an effective amount of a composition described herein or an effective amount of at least isolated nucleic acid molecule described herein.

In one embodiment, the hematopoietic progenitor or stem cells or iPSCs or isolated cells are autologous to the mammal, meaning the cells are derived from the same mammal. In another of the embodiments of the described method, the hematopoietic progenitor or stem cells or iPSCs or isolated cells are non-autologous to the mammal, meaning the cells are not derived from the same mammal, but another mammal of the same species. For example, the mammal is a human.

In one embodiment of this aspect and all other aspects described herein, the contacted progenitor cell or contacted cell acquires at least one epigenetic modification in the BCL11A enhancer functional region.

In one embodiment of this aspect and all other aspects described herein, the at least one epigenetic modification is selected from the group consisting of alteration of DNA methylation, histone tail modification, histone subunit composition and nucleosome positioning.

In one embodiment of this aspect and all other aspects described herein, the at least one epigenetic modification in the genomic DNA of the cell on chromosome 2 indirectly or directly affects the location 60725424 to 60725688 (+55 functional region), and/or at location 60722238 to 60722466 (+58 functional region), and/or at location 60718042 to 60718186 (+62 functional region), or at location 60773106 to 60773435 (exon 2) of chromosome 2.

In one embodiment of this aspect and all other aspects described herein, the at least one epigenetic modification is located between chromosome 2 location 60725424 to 60725688 (+55 functional region), at location 60722238 to 60722466 (+58 functional region), and/or at location 60718042 to 60718186 (+62 functional region), or at location 60773106 to 60773435 (exon 2) of the human chromosome 2 is that according to UCSC Genome Browser hg 19 human genome assembly.

As used herein, “indirectly affecting the location 60725424 to 60725688 (+55 functional region), at location 60722238 to 60722466 (+58 functional region), and/or at location 60718042 to 60718186 (+62 functional region) of chromosome 2” refers to long distance effects of epigenetic modification in the genomic DNA of the cell on chromosome 2 the location 60725424 to 60725688 (+55 functional region), at location 60722238 to 60722466 (+58 functional region), or at location 60718042 to 60718186 (+62 functional region), or at location 60773106 to 60773435 (exon 2) of chromosome 2.

In one embodiment of any one aspects described herein, the contacted cells or the differentiated progeny cells therefrom further have increased fetal hemoglobin levels.

In one embodiment of any one aspects described herein, the contacted cells or the differentiated progeny cells therefrom further have decreased BCL11A mRNA or protein expression compared to non-contacted control cells.

In one embodiment of any one aspects described herein, the isolated genetic engineered human cell or a population of genetic engineered human cells described herein or the differentiated progeny cells therefrom have increased fetal hemoglobin levels.

In one embodiment of any one aspects described herein, the genetically edited progenitor cells described herein or the differentiated progeny cells therefrom have increased fetal hemoglobin levels.

In one embodiment, the isolated genetic engineered human cell or a population of genetic engineered human cells described herein or the differentiated progeny cells therefrom have decreased BCL11A mRNA or protein expression compared to non-contacted control cells.

In one embodiment, the genetically edited progenitor cells described herein or the differentiated progeny cells therefrom have decreased BCL11A mRNA or protein expression compared to non-contacted control cells.

In one embodiment, the isolated cell or isolated population of cells used in the contacting, the methods described herein, or electroporation described herein is/are human or progenitor cell(s). In another embodiment, the hematopoietic progenitor cell, the isolated human cell, or isolated cell is contacted ex vivo or in vitro.

In another embodiment, the at least one genetic modification is a deletion. In another embodiment of this aspect and all other aspects described herein, the at least one epigenetic modification. In another embodiment, the deletion comprises one or more of the DNAse 1-hypersensitive sites (DHS) +62, +58, or +55, or exon 2.

In another embodiment, the epigenetic modification comprises or affects one or more of the DNAse 1-hypersensitive sites (DHS) +62, +58, and +55. As used herein, the phrase “affects one or more of the DNAse 1-hypersensitive sites” means natural function of these DNAse 1-hypersensitive sites (DHS) +62, +58, and +55 are reduce, for example, access to transcription factors or DNA degradation enzymes such as DNase I. In general, DNase I hypersensitive sites (DHSs) are regions of chromatin which are sensitive to cleavage by the DNase I enzyme. In these specific regions of the genome, chromatin has lost its condensed structure, exposing the DNA, and making it accessible. This raises the availability of DNA to degradation by enzymes, like DNase I. These accessible chromatin zones are functionally related to transcriptional activity, since this remodeled state is necessary for the binding of proteins such as transcription factors. Accordingly, the epigenetic modification contemplated herein results in reduced access to DNA degradation enzymes that is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated in any method disclosed herein.

In another embodiment, the epigenetic modification is from 60,716,189 to 60,728,612, from 60,716,189 to 60,723,870, from 60,722,992 to 60,728,612, from 60,717,236 to 60,719,036, from 60,722,006 to 60,723,058, from 60,724,917 to 60,726,282, from 60,616,396 to 60,618,032, from 60,623,536 to 60,624,989, from 60,626,565 to 60,628,177, from 60,717,236 to 60,719,036, from 60,721,212 to 60,722,958, from 60,724,780 to 60,726,471, from 60,739,075 to 60,740,154, from 60,748,003 to 60,749,009, from 60,826,438 to 60,827,601, or from 60,831,589 to 60,833,556.

In another embodiment, the epigenetic modification that interferes with the establishment and/or maintenance of the epigenetic signature at the enhancer region on chromosome 2 location 60725424 to 60725688 (+55 functional region), and/or at location 60722238 to 60722466 (+58 functional region), and/or at location 60718042 to 60718186 (+62 functional region) (according to UCSC Genome Browser hg 19 human genome assembly) thereby leading to reduced mRNA or protein expression of BCL11A, and increasing fetal hemoglobin expression in the mammal.

In one embodiment, the epigenetic modification that interferes with the establishment and/or maintenance of the epigenetic signature at the enhancer region on chromosome 2 location 60725424 to 60725688 (+55 functional region), and/or at location 60722238 to 60722466 (+58 functional region), and/or at location 60718042 to 60718186 (+62 functional region) (according to UCSC Genome Browser hg 19 human genome assembly) includes but is not limited to epigenetic modifications that affects DNase I sensitivity, epigenetic modifications that affects histone modifications, epigenetic modifications that affects GATA 1/TAL 1 binding, and epigenetic modifications that affects long-range promoter interaction of the promoter of BCL11A.

For example, an epigenetic modification that interferes with the establishment and/or maintenance of the epigenetic signature at the enhancer region on chromosome 2 location functional regions described include but is not limited to at least one deletion within chromosome 2 location 60,716,189-60,728,612 such that the overall function of this region is affected whereby the mRNA and expression of BCL11A is reduced or decreased. For example, the deletion is at the DNasel sensitivity regions chromosome 2 location 60,716,189-60,728,612, e.g., +62, +58, and +55. The deletion could be at +62 or +58 or +55 or combination thereof. For examples, at +62 and +58, +58 and +55, +62 and +55, or at all three +62, +58, and +55.

As another example, an epigenetic modification that interferes with the establishment and/or maintenance of the epigenetic signature at the enhancer region on chromosome 2 location +55, +58 and +62 functional regions include but is not limited to changes in the histone modifications on chromosome 2 that is not at location functional regions or changes in the histone modifications on chromosome 2 at location functional regions, or both histone modifications on chromosome 2 not at location 60,716,189-60,728,612 as well as at location 60,716,189-60,728,612 such that the overall function of this region is affected whereby the mRNA and expression of BCL11A is reduced or decreased.

In another embodiment, an epigenetic modification that interferes with the establishment and/or maintenance of the epigenetic signature at the enhancer region on chromosome 2 location 60,716,189-60,728,612 include but is not limited to an insertion of at least one engineered specific-repressor sequence that change the epigenetic features of noncoding elements at chromosome 2, +55, +58 and +62 functional regions, and thus result in repression of target gene expression. The first is specifically focused on epigenetically repressing individual enhancers. In other words, insertion of engineered specific-repressor sequences into chromosome 2 would prospectively interfering with epigenetic modification at the BCL11A erythroid enhancer which eventually leads to reduced BCL11A gene expression.

Any methods known in the art can be used to produce the epigenetic modification contemplated. For example, as described in Mendenhall E M. et al., Nat. Biotechnol. 8 Sep. 2013, and Maeder M L et al., Nat Biotechnol. 9 Oct. 2013.

In one embodiment, the insertion of at least one engineered specific-repressor sequence on any location chromosome 2 results in but is not limited to reduced DNasel sensitivity regions at chromosome 2 location +55, +58 and +62 functional regions; increased histone modifications on chromosome 2 location 60,716,189-60,728,612 or at the +55, +58 and +62 functional regions; reduced transcription factors binding to the GATA1/TAL1 of the enhancer region on chromosome 2 +55, +58 and +62 functional regions; and reduced or weakened interaction between the chromosome 2 location +55, +58 and +62 functional regions with the BCL11A promoter.

In one embodiment, the overall effects of the insertion of at least one engineered specific-repressor sequence on any location chromosome 2 is reduced or decreased mRNA and expression of BCL11A.

In some embodiments, as used in the context of mRNA and expression of BCL11A, interaction between the chromosome 2 location 60,716,189-60,728,612, at the +55, +58 and +62 functional regions, or BCL11A enhancer with the BCL11A promoter, and transcription factors binding to the GATA1/TAL1 of the enhancer region, the term “reduced” or “decreased” refers to at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to the control situation that is in the absence of the epigenetic modification or genetic modification or insertion of engineered sequences disclosed herein. By decrease of the BCL11A mRNA or protein expression in the cell means that protein expression is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that does not have the epigenetic modification or genetic modification or insertion of engineered sequences disclosed herein.

In one embodiment of this aspect, the insertion of at least one engineered specific-repressor sequence occurs within the DNasel sensitivity regions of chromosome 2 at location 60,716,189-60,728,612, or at the +55, +58 and +62 functional regions or at exon 2. The insertion could be at the 5′end of +62 or +58 or +55 or at the 3′end of +62 or +58 or +55, or between +62 and +58, or between +58 and +55, or between +55 and +62.

In one embodiment of this aspect, the insertion of at least one engineered specific-repressor sequence changes the DNasel sensitivity regions of chromosome 2 at location +55, +58 and +62 functional regions.

In one embodiment, the epigenetic modifications change the DNasel sensitivity regions of chromosome 2 at location 60,716,189-60,728,612 or at the +55, +58 and +62 functional regions. In one embodiment, the epigenetic modifications change the histone modifications on chromosome 2 location 60,716,189-60,728,612, or at the +55, +58 and +62 functional regions.

In one embodiment, the insertion of at least one engineered specific-repressor sequence changes the histone modifications on chromosome 2 location 60,716,189-60,728,612 or at the +55, +58 and +62 functional regions.

In one embodiment, the epigenetic modifications change the GATA1/TAL1 binding of the enhancer region on chromosome +55, +58 and +62 functional regions, such that the overall function of this region is affected whereby the mRNA and expression of BCL11A is reduced or decreased. For example, the binding of transcription factors to the GATA1/TAL 1.

In one embodiment, the genetic modification such as an insertion or deletion or substitution occurs within the GATA1/TAL1 as described herein. The insertion can be at the 5′ end or 3′end of GATA1 or TAL 1. The genetic modification can be between GATA1 and TAL 1. The genetic modification changes the GATA1/TAL1 binding of the enhancer region on chromosome 2 +55, +58 and +62 functional regions, such that the overall function of this region is affected whereby the mRNA and expression of BCL11A is reduced or decreased. For example, the binding of transcription factors to the GATA1/TAL1.

In one embodiment, the epigenetic modification and/or genetic modification changes the interaction between the BCL11A enhancer and the BCL11A promoter. In one embodiment, the interaction is reduced or weakened such that the overall function of this region is affected whereby the mRNA and expression of BCL11A is reduced or decreased.

In one embodiment, the epigenetic modifications and/or genetic modification change the interaction between the chromosome 2 location 60,716,189-60,728,612 and/or the +55, +58 and +62 functional regions with the BCL11A promoter. In one embodiment, the interaction is reduced or weakened such that the overall function of this region is affected whereby the mRNA and expression of BCL11A is reduced or decreased.

In one embodiment, the isolated genetic engineered human cell has at least one epigenetic modification at the genomic DNA of the cell on chromosome 2. In another of this aspect, the isolated genetic engineered human cell has at least one epigenetic modification at the genomic DNA of the cell on chromosome 2 location 60725424 to 60725688 (+55 functional region), and/or at location 60722238 to 60722466 (+58 functional region), and/or at location 60718042 to 60718186 (+62 functional region) or at the BCL11A exon2.

In some aspects of any of these isolated genetic engineered human cells having at least one epigenetic modification or genetic modification, the cells are transplanted into a mammal for use in increasing the fetal hemoglobin in the mammal.

In one embodiment of this aspect and all other aspects described herein, the isolated genetic engineered human cell having at least one genetic modification at the genomic DNA of the cell on chromosome 2 location 60725424 to 60725688 (+55 functional region), and/or at location 60722238 to 60722466 (+58 functional region), and/or at location 60718042 to 60718186 (+62 functional region) or at the BCL11A exon 2 is transplanted into a mammal for use in increasing the fetal hemoglobin in the mammal.

In one embodiment of this aspect and all other aspects described herein, the isolated genetic engineered human cell having at least one genetic modification at the genomic DNA of the cell on chromosome 2 location 60725424 to 60725688 (+55 functional region), and/or at location 60722238 to 60722466 (+58 functional region), and/or at location 60718042 to 60718186 (+62 functional region) or at the BCL11A exon 2 is stored for later use by cryopreservation.

In some aspects of any of those isolated genetic engineered human cells having at least one epigenetic modification or genetic modification, the cells are stored for later use by cryopreservation.

In one embodiment of this aspect and all other aspects described herein, the isolated genetic engineered human cell having at least one genetic modification at the genomic DNA of the cell on chromosome 2 location 60725424 to 60725688 (+55 functional region), and/or at location 60722238 to 60722466 (+58 functional region), and/or at location 60718042 to 60718186 (+62 functional region) or at the BCL11A exon 2 is cryopreserved, thawed and transplanted into mammal for use in increasing the fetal hemoglobin in the mammal.

In some aspects of any of those isolated genetic engineered human cells having at least one epigenetic modification or genetic modification, cryopreserved, thawed and transplanted into mammal for use in increasing the fetal hemoglobin in the mammal.

In one embodiment of this aspect and all other aspects described herein, the composition causes an increase in fetal hemoglobin mRNA or protein expression in the contact cell.

In one embodiment of this aspect and all other aspects described herein, the cells of any compositions described are autologous, to the mammal who is the recipient of the cells in a transplantation procedure, i.e., the cells of the composition are derived or harvested from the mammal prior to any described genetic modification or targeted gene editing.

In one embodiment of this aspect and all other aspects described herein, the cells of any compositions described are non-autologous to the mammal who is the recipient of the cells in a transplantation procedure, i.e., the cells of the composition are not derived or harvested from the mammal prior to any described genetic modification or targeted gene editing.

In one embodiment of this aspect and all other aspects described herein, the cells of any compositions described are at the minimum HLA type matched with to the mammal who is the recipient of the cells in a transplantation procedure.

In one embodiment of this aspect and all other aspects described herein, the cells of any compositions described are isolated progenitor cells prior to any described genetic modification or targeted gene editing.

In one embodiment of this aspect and all other aspects described herein, the cells of any compositions described are isolated hematopoietic progenitor cells prior to any described genetic modification or targeted gene editing.

In one embodiment of this aspect and all other aspects described herein, the cells of any compositions described are isolated induced pluripotent stem cells prior to any described genetic modification or targeted gene editing.

In another embodiment of this aspect and all other aspects described herein, the deletion comprises one or more of the DNAse 1-hypersensitive sites (DHS) +62, +58, and +55. In another embodiment of this aspect and all other aspects described herein, the deletion consists essentially of one or more of the DNAse 1-hypersensitive sites (DHS) +62, +58, and +55. In another embodiment, the deletion consists of one or more of the DNAse 1-hypersensitive sites (DHS) +62, +58, and +55. In one embodiment, as used herein, the term “portion” in the context of genomic deletion is at least 10% to about 100% of the specified region. In other embodiments, the portion deleted is at least 20%, at least 30%, at least 40%, at least 50%, at least 60%, at least 70%, at least 80%, at least 90%, at least 95%, at least 99% or even 100% of the specified region.

In one embodiment of this aspect and all other aspects described herein, the method further comprises selecting a mammal in need of increasing fetal hemoglobin, such as a mammal having a hemoglobinopathy.

In one embodiment of this aspect and all other aspects described herein, the mammal has been diagnosed with a hemoglobinopathy.

In one embodiment of this aspect and all other aspects described herein, the mammal in need of increasing fetal hemoglobin has been diagnosed with a hemoglobinopathy.

In one embodiment of this aspect and all other aspects described herein, the hemoglobinopathy is a β-hemoglobinopathy.

In one embodiment of this aspect and all other aspects described herein, the hemoglobinopathy is sickle cell disease.

In one embodiment of this aspect and all other aspects described herein, the hemoglobinopathy is β-thalassemia.

In one embodiment of this aspect and all other aspects described herein, the contacted cell, human cell, hematopoietic progenitor cell or its progeny is administered to the mammal.

In further embodiment of any one treatment method described, the method comprises chemotherapy and/or radiation therapy to remove or reduced the endogenous hematopoietic progenitor or stem cells in the mammal.

In one embodiment of any one method described herein, the contacted cells, targeted gene edited cells described herein having at least one genetic modification can be cryopreserved and stored until the cells are needed for administration into a mammal.

In one embodiment of any one method described herein, the contacted cells, targeted gene edited cells described herein having at least one genetic modification can be cultured ex vivo to expand or increase the number of cells prior to storage, e.g. by cryopreservation, or prior to use, e.g., transplanted into a recipient mammal, e.g., a patient.

In one embodiment of this aspect and all other aspects described herein, the contacted population of hematopoietic progenitor or stem cells having increased fetal hemoglobin expression is cryopreserved and stored or reintroduced into the mammal. In another embodiment, the cryopreserved population of hematopoietic progenitor or stem cells having increased fetal hemoglobin expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs described herein.

In another embodiment, the cryopreserved population of hematopoietic progenitor or stem cells having increased fetal hemoglobin expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs derived from the mammal. In any embodiment of the method, the method further comprises selecting a mammal in need of increased fetal hemoglobin expression.

In further embodiment of this method, the population of hematopoietic progenitor or stem cells with genetic modification or targeted gene editing in the genomic DNA and having increased fetal hemoglobin expression is cryopreserved and stored or reintroduced into the mammal. In another embodiment, the population of hematopoietic progenitor or stem cells with deleted genomic DNA and having increased fetal hemoglobin expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs described herein. In any of the embodiment of the described method, the hematopoietic progenitor or stem cells or iPSCs are analogous to the mammal, meaning the cells are derived from the same mammal. In another of the embodiment of the described method, the hematopoietic progenitor or stem cells or iPSCs are non-analogous to the mammal, meaning the cells are not derived from the same mammal, but another mammal of the same species. For example, the mammal is a human. In any embodiment of the method, the method further comprises selecting a mammal in need of increased fetal hemoglobin expression.

In one embodiment, the population of hematopoietic progenitor or stem cells with genetic modification or targeted gene editing in the genomic DNA and having increased fetal hemoglobin expression is cryopreserved and stored or reintroduced into the mammal. In another embodiment, the cryopreserved population of hematopoietic progenitor or stem cells having increased fetal hemoglobin expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs derived from the mammal. In any embodiment of the method, the method further comprises selecting a mammal in need of increased fetal hemoglobin expression.

In one embodiment, the method further comprises selecting a mammal in need of increased fetal hemoglobin expression. Exemplary mammal in need of increased fetal hemoglobin expression is one that has been diagnosed with a hemoglobinopathy.

In one embodiment, the cells obtained after electroporation the compositions described herein can be cryopreserved till they are needed for administration into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells or iPSCs are autologous to the mammal, meaning the cells are derived from the same mammal. In another of the embodiment of the described method, the hematopoietic progenitor or stem cells or iPSCs are non-autologous to the mammal, meaning the cells are not derived from the same mammal, but another mammal of the same species. For example, the mammal is a human.

In one embodiment, the method further comprises selecting a mammal in need of treatment of a hemoglobinopathy.

In one embodiment, the cells obtained after the contacting step can be cryopreserved till they are needed for administration into the mammal. In any embodiment of the method, the method further comprises selecting a mammal in need of treatment of a hemoglobinopathy.

In one embodiment, the method further comprises administering the cells obtained after the contacting step into the mammal.

In one embodiment, the method further comprises of selecting a subject diagnosed with a hemoglobinopathy or a subject at risk of developing a hemoglobinopathy.

In one embodiment, the hemoglobinopathy is sickle cell disease (SCD) or thalassemia (THAL). For example, β-thalassemias.

In one embodiment, the method further comprising administering to the subject a therapy comprising oxygen, hydroxyurea, folic acid, or a blood transfusion.

In one aspect, the present specification provides a method of treating, or reducing a risk of developing, a hemoglobinopathy in a subject. In any embodiment of any treatment method described, the hemoglobinopathy is a β-hemoglobinopathy, β-thalassemia, or sickle cell anemia.

In one of embodiment of any described method, the hematopoietic progenitor or stem cells or iPSCs are autologous to the mammal, meaning the cells are derived from the same mammal. In another of the embodiment of any described method, the hematopoietic progenitor or stem cells or iPSCs are non-autologous to the mammal, meaning the cells are not derived from the same mammal, but another mammal of the same species. For example, the mammal is a human.

In one of embodiment of any described method, the contacting of any cell described herein can be ex vivo or in vitro or in vivo.

In one aspect, fetal hemoglobin expression is increased in the mammal, relative to expression prior to the contacting.

In another embodiment of any described method, the hematopoietic progenitor cell, the isolated human cell, or isolated cell is contacted ex vivo or in vitro.

In another embodiment of any described method, the at least one genetic modification is a deletion. In another embodiment of this aspect and all other aspects described herein, the at least one epigenetic modification.

In one embodiment of use of the composition described herein, the composition causes an increase in fetal hemoglobin mRNA or protein expression in the contact cell.

In one embodiment of use of the composition described herein, the cells of any compositions described are autologous, to the mammal who is the recipient of the cells in a transplantation procedure, i.e., the cells of the composition are derived or harvested from the mammal prior to any described modification.

In one embodiment of use of the composition described herein, the cells of any compositions described are non-autologous to the mammal who is the recipient of the cells in a transplantation procedure, i.e., the cells of the composition are not derived or harvested from the mammal prior to any described modification.

In one embodiment of use of the composition described herein, the cells of any compositions described are at the minimum HLA type matched with to the mammal who is the recipient of the cells in a transplantation procedure.

In one embodiment of use of the composition described herein, the cells of any compositions described are isolated progenitor cells prior to any described genetic modification by targeted editing using the methods described herein.

In one embodiment of use of the composition described herein, the cells of any compositions described are isolated hematopoietic progenitor cells prior to any described genetic modification by targeted editing using the methods described herein.

In one embodiment of use of the composition described herein, the cells of any compositions described are isolated induced pluripotent stem cells prior to any described genetic modification by targeted editing using the methods described herein.

In one embodiment of use of the composition described herein, the cells of any compositions described are cryopreserved prior to use.

In one embodiment of any one method described, the method is used to treat, prevent, or ameliorate a hemoglobinopathy is selected from the group consisting of: hemoglobin C disease, hemoglobin sickle cell disease (SCD), sickle cell anemia, hereditary anemia, thalassemia, β-thalassemia, thalassemia major, thalassemia intermedia, a-thalassemia, and hemoglobin H disease.

The contacted or electroporated cells described herein are then administered to a subject in need of gene therapy.

In one embodiment of any one method described, the method further comprises selecting a subject in need of the gene therapy described. For example, a subject exhibiting symptoms or cytology of a hemoglobinopathy is selected from the group consisting of hemoglobin C disease, hemoglobin sickle cell disease (SCD), sickle cell anemia, hereditary anemia, thalassemia, β-thalassemia, thalassemia major, thalassemia intermedia, a-thalassemia, and hemoglobin H disease. Alternatively, the subject carries a genetic mutation that is associated with a hemoglobinopathy, a genetic mutation described herein. For example, a subject diagnosis of SCD with genotype HbSS, HbS/β0 thalassemia, HbSD, or HbSO, and/or with HbF <10% by electrophoresis.

In a particular embodiment, a method of preventing, ameliorating, or treating a hemoglobinopathy in a subject is provided. The method comprises administering a population of cells comprising engineered/genetically modified hematopoietic stem cells or hematopoietic progenitor cells described herein.

In particular embodiments of any methods described, a population of engineered/genetically modified cells administered to a subject comprises hematopoietic stem or progenitor cells, proerythroblasts, basophilic erythroblasts, polychromatic erythroblasts, orthochromatic erythroblasts, polychromatic erythrocytes, and erythrocytes (RBCs), or any combination thereof

In some embodiments of any methods described, the population of engineered/genetically modified cells can be culture expanded in vitro or ex vivo prior to implantation/engraftment into a subject or prior to cryopreservation for storage.

In some embodiments of any methods described, the population of engineered/genetically modified cells can be culture expanded in vitro or ex vivo after cryopreservation prior to implantation/engraftment into a subject.

In some embodiments of any methods described, the population of engineered/genetically modified cells can be differentiated in vitro or ex vivo prior to implantation into a subject.

The genetically modified cells may be administered as part of a bone marrow or cord blood transplant in an individual that has or has not undergone bone marrow ablative therapy. In one embodiment, genetically modified cells contemplated herein are administered in a bone marrow transplant to an individual that has undergone chemoablative or radioablative bone marrow therapy.

In one embodiment of any method described, a dose of genetically modified cells is delivered to a subject intravenously. In one embodiment, genetically modified hematopoietic cells are intravenously administered to a subject.

In particular embodiments, patients receive a dose of genetically modified cells, e.g., hematopoietic stem cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10′ cells/kg, about 5×10′ cells/kg, about 1×10⁸ cells/kg, or more in one single intravenous dose. In certain embodiments, patients receive a dose of genetically modified cells, e.g., hematopoietic stem cells described herein or genetic engineered cells described herein or progeny thereof, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In an additional embodiment, patients receive a dose of genetically modified cells, e.g., hematopoietic stem cells, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8 x 10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods described here provide more robust and safe gene therapy than existing methods and comprise administering a population or dose of cells comprising about 5% transduced/genetically modified cells, about 10% transduced/genetically modified cells, about 15% transduced/genetically modified cells, about 20% transduce/genetically modified d cells, about 25% transduced/genetically modified cells, about 30% transduced/genetically modified cells, about 35% transduced/genetically modified cells, about 40% transduced/genetically modified cells, about 45% transduced/genetically modified cells, or about 50% transduce/genetically modified cells, to a subject.

In one embodiment, the invention provides genetically modified cells, such as a stem cell, e.g., hematopoietic stem cell, with the potential to expand or increase a population of erythroid cells. Hematopoietic stem cells are the origin of erythroid cells and thus, are preferred.

In one embodiment, the contacted hematopoietic stem cells described herein or genetic engineered cells described herein or the progeny cells thereof are implanted with prostaglandin E2 and/or antioxidant N-acetyl-L-cysteine (NAC) to promote the engraftments of the respective cells.

In a further embodiment of any methods described herein, the hematopoietic stem cell or hematopoietic progenitor cell being contacted is of the erythroid lineage.

In one embodiment of any methods described herein, the hematopoietic stem cell or hematopoietic progenitor cell is collected from peripheral blood, cord blood, chorionic villi, amniotic fluid, placental blood, or bone marrow.

In a further embodiment of any methods described herein, the recipient subject is treated with chemotherapy and/or radiation prior to implantation of the contacted or transfected cells (i.e. the contacted hematopoietic stem cells described herein or genetic engineered cells described herein or the progeny cells thereof).

In one embodiment, the chemotherapy and/or radiation is to reduce endogenous stem cells to facilitate engraftment of the implanted cells.

In one aspect of any method, the contacted hematopoietic stem cells described herein or genetic engineered cells described herein or the progeny cells thereof are treated ex vivo with prostaglandin E2 and/or antioxidant N-acetyl-L-cysteine (NAC) to promote subsequent engraftment in a recipient subject.

Engraftment analysis was performed 4, 8 and 12 weeks post transplantation in peripheral blood and bone marrow. For example, harvest a sample of blood from these locations and determine the BCL11A expression by any method known in the art.

In one aspect of any one method described herein, the method comprises obtaining a sample or a population of embryonic stem cells, somatic stem cells, progenitor cells, bone marrow cells, hematopoietic stem cells, or hematopoietic progenitor cells from the subject.

In one embodiment of any one method described herein, the cells that is contacted with a nucleic acid molecule describe herein, or a composition describe herein comprising a nucleic acid molecule.

In one embodiment, the embryonic stem cells, somatic stem cells, progenitor cells, bone marrow cells, hematopoietic stem cells, hematopoietic progenitor cells are isolated from the host subject, transfected, cultured (optional), and transplanted back into the same host, i. e. an autologous cell transplant. In another embodiment, the embryonic stem cells, somatic stem cells, progenitor cells, bone marrow cells, hematopoietic stem cells, or hematopoietic progenitor cells are isolated from a donor who is an HLA-type match with a host (recipient) who is diagnosed with or at risk of developing a hemoglobinopathy. Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different host, i.e., allogeneic to the recipient host subject. The donor's or subject's embryonic stem cells, somatic stem cells, progenitor cells, bone marrow cells, hematopoietic stem cells, or hematopoietic progenitor cells can be contacted (electroporated) with a nucleic acid molecule described herein, the contacted cells are culture expanded, and then transplanted into the host subject. In one embodiment, the transplanted cells engraft in the host subject. The transfected cells can also be cryopreserved after transfected and stored, or cryopreserved after cell expansion and stored.

In one aspect of any method, the embryonic stem cell, somatic stem cell, progenitor cell, bone marrow cell, hematopoietic stem cell, or hematopoietic progenitor cell is autologous or allogeneic to the subject.

### Hematopoietic Progenitor Cells

In one embodiment, the hematopoietic progenitor cell is contacted ex vivo or in vitro. In a specific embodiment, the cell being contacted is a cell of the erythroid lineage. In one embodiment, the cell composition comprises cells having decreased BCL11A expression.

“Hematopoietic progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiment, the hematopoietic progenitor cell has at least one of the cell surface marker characteristic of hematopoietic progenitor cells: CD34+, CD59+, Thyl/CD90+, CD381o/−, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter119.

Stem cells, such as hematopoietic progenitor cells, are capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential.

Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

### Induced Pluripotent Stem Cells

In some embodiments, the genetic engineered human cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described induced pluripotent stem cells. Yamanaka and Takahashi converted mouse somatic cells to ES cell-like cells with expanded developmental potential by the direct transduction of Oct4, Sox2, Klf4, and c-Myc (Takahashi and Yamanaka, 2006). iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5;7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-¾, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (-)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-CI-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Somatic Cells for Reprogramming

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nati); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2;β3-catenin, and Bmi1. Such cells can also be characterized by the down-regulation of markers characteristic of the somatic cell from which the induced pluripotent stem cell is derived.

### Pharmaceutically Acceptable Carriers

The methods of administering human hematopoietic progenitor cells or genetic engineered cells described herein or their progeny to a subject as described herein involve the use of therapeutic compositions comprising hematopoietic progenitor cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the hematopoietic progenitor cells described herein or genetic engineered cells described herein or their progeny are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the hematopoietic progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, the compositions of isolated genetic engineered cells described further comprises a pharmaceutically acceptable carrier. In one embodiment, the pharmaceutically acceptable carrier does not include tissue or cell culture media.

In some embodiments, the compositions of modified synthetic nucleic acid molecules described further comprises a pharmaceutically acceptable carrier. In one embodiment, the pharmaceutically acceptable carrier does not include tissue or cell culture media.

In some embodiments, the compositions comprising the nucleic acid molecules described further comprises a pharmaceutically acceptable carrier. In one embodiment, the pharmaceutically acceptable carrier does not include tissue or cell culture media.

### Administration & Efficacy

As used herein, the terms “administering,” “introducing” and “transplanting” are used interchangeably in the context of the placement of cells, e.g. hematopoietic progenitor cells, as described herein into a subject, by a method or route which results in at least partial localization of the introduced cells at a desired site, such as a site of injury or repair, such that a desired effect(s) is produced. The cells e.g. hematopoietic progenitor cells, or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of hematopoietic progenitor cells or engineered cells with reduced BCL11A expression is administered via a systemic route of administration, such as an intraperitoneal or intravenous route.

When provided prophylactically, hematopoietic progenitor cells or engineered cells with reduced BCL11A expression described herein can be administered to a subject in advance of any symptom of a hemoglobinopathy, e.g., prior to the switch from fetal γ-globin to predominantly β-globin. Accordingly, the prophylactic administration of a hematopoietic progenitor cell population serves to prevent a hemoglobinopathy, as disclosed herein.

When provided therapeutically, hematopoietic progenitor cells are provided at (or after) the onset of a symptom or indication of a hemoglobinopathy, e.g., upon the onset of sickle cell disease.

In some embodiments of the aspects described herein, the hematopoietic progenitor cell population or engineered cells with reduced BCL11A expression being administered according to the methods described herein comprises allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic progenitor cell population or engineered cells with reduced BCL11A expression being administered to a subject can be derived from umbilical cord blood obtained from one more unrelated donor subjects, or from one or more non-identical siblings. In some embodiments, syngeneic hematopoietic progenitor cell populations can be used, such as those obtained from genetically identical animals, or from identical twins. In other embodiments of this aspect, the hematopoietic progenitor cells are autologous cells; that is, the hematopoietic progenitor cells are obtained or isolated from a subject and administered to the same subject, i.e., the donor and recipient are the same.

For use in the various aspects described herein, an effective amount of hematopoietic progenitor cells or engineered cells with reduced BCL11A expression, comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ hematopoietic progenitor cells, at least 7×10⁵ cells, at least 8×10⁵ cells, at least 9×10⁵ cells, at least 1 X 10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The hematopoietic progenitor cells or engineered cells with reduced BCL11A expression can be derived from one or more donors, or can be obtained from an autologous source. In some embodiments of the aspects described herein, the hematopoietic progenitor cells are expanded in culture prior to administration to a subject in need thereof

In one embodiment, the term “effective amount” as used herein refers to the amount of a population of human hematopoietic progenitor cells or their progeny needed to alleviate at least one or more symptom of a hemoglobinopathy, and relates to a sufficient amount of a composition to provide the desired effect, e.g., treat a subject having a hemoglobinopathy. The term “therapeutically effective amount” therefore refers to an amount of hematopoietic progenitor cells, or genetic engineered cells described herein or their progeny or a composition comprising hematopoietic progenitor cells, or genetic engineered cells described herein or their progeny that is sufficient to promote a particular effect when administered to a typical subject, such as one who has or is at risk for a hemoglobinopathy. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery of a hematopoietic stem cell composition as described herein into a subject by a method or route which results in at least partial localization of the cell composition at a desired site. A cell composition can be administered by any appropriate route which results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. For the delivery of cells, administration by injection or infusion is generally preferred.

In one embodiment, the cells as described herein are administered systemically. The phrases “systemic administration,” “administered systemically”, “peripheral administration” and “administered peripherally” as used herein refer to the administration of a population of hematopoietic progenitor cells other than directly into a target site, tissue, or organ, such that it enters, instead, the subject's circulatory system and, thus, is subject to metabolism and other like processes.

The efficacy of a treatment comprising a composition as described herein for the treatment of a hemoglobinopathy can be determined by the skilled clinician. However, a treatment is considered “effective treatment,” as the term is used herein, if any one or all of the signs or symptoms of, as but one example, levels of fetal β-globin are altered in a beneficial manner, other clinically accepted symptoms or markers of disease are improved or ameliorated, e.g., by at least 10% following treatment with an inhibitor. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of sepsis; or (2) relieving the disease, e.g., causing regression of symptoms; and (3) preventing or reducing the likelihood of the development of infection or sepsis.

The treatment according to the present invention ameliorates one or more symptoms associated with a β-globin disorder by increasing the amount of fetal hemoglobin in the individual. Symptoms typically associated with a hemoglobinopathy, include for example, anemia, tissue hypoxia, organ dysfunction, abnormal hematocrit values, ineffective erythropoiesis, abnormal reticulocyte (erythrocyte) count, abnormal iron load, the presence of ring sideroblasts, splenomegaly, hepatomegaly, impaired peripheral blood flow, dyspnea, increased hemolysis, jaundice, anemic pain crises, acute chest syndrome, splenic sequestration, priapism, stroke, hand-foot syndrome, and pain such as angina pectoris.

In one embodiment, the hematopoietic progenitor cell is contacted ex vivo or in vitro with a DNA targeting endonuclease, and the cell or its progeny is administered to the mammal (e.g., human). In a further embodiment, the hematopoietic progenitor cell is a cell of the erythroid lineage. In one embodiment, a composition comprising a hematopoietic progenitor cell that was previously contacted with a DNA-targeting endonuclease and a pharmaceutically acceptable carrier and is administered to a mammal.

In one embodiment, any method known in the art can be used to measure an increase in fetal hemoglobin expression, e.g., Western Blot analysis of fetal hemoglobin protein and quantifying mRNA of fetal γ-globin.

In one embodiment, the hematopoietic progenitor cell is contacted with a DNA-targeting endonuclease in vitro, or ex vivo. In one embodiment, the cell is of human origin (e.g., an autologous or heterologous cell). In one embodiment, the composition causes an increase in fetal hemoglobin expression.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Furthermore, the disclosure described herein does not concern the destruction of a human embryo.

This invention is further illustrated by the following example which should not be construed as limiting. The contents of all references cited throughout this application, as well as the figures and table are incorporated herein by reference.

The invention can further be described in any one of the following numbered paragraphs.

1) A ribonucleoprotein (RNP) complex comprising:

a. a base editor protein; and

b. a nucleic acid sequence shown in Table 1, SEQ ID NOS: 1-139, wherein there is at least one chemical modification to a nucleotide in the nucleic acid sequence.

2) The RNP complex of paragraph 1, wherein the nucleic acid sequence excludes the entire BCL11A enhancer functional regions and excludes the entire BCL11A coding region.

3) The RNP complex of any preceding paragraph, wherein the nucleic acid sequence includes the entire BCL11A enhancer functional regions and excludes the entire BCL11A coding region.

4) The RNP complex of any preceding paragraph, wherein the nucleic acid sequence is selected from the group consisting of SEQ ID NOS: 37, 38, 39, 40, 41, 42, 43, 44, 45, 46, 47, 48, 50, 51, 52, 53, 54, 55, 56, 57, and 62 as shown in Table 2.

5) The RNP complex of any preceding paragraph, wherein the chemical modification is located at one or more terminal nucleotides in nucleic acid sequence.

6) The RNP complex of any preceding paragraph, wherein the chemical modification is selected from the group consisting of 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, 0, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag).

7) The RNP complex of any preceding paragraph, wherein the chemical modification is located only at the 3′ end, or added only at the 5′ end, or added at both the 5′ and 3′ ends of the nucleic acid sequence.

8) The RNP complex of any preceding paragraph, wherein the chemical modification is located to first three nucleotides and to the last three nucleotides of the nucleic acid sequence.

9) The RNP complex of any preceding paragraph, wherein the nucleic acid sequence further comprising a crRNA/tracrRNA sequence.

10) The RNP complex of any preceding paragraph, wherein the nucleic acid sequence is a single guide RNA (sgRNA).

11) The RNP complex of any preceding paragraph, wherein the nucleic acid sequence excludes the entire region between the human chromosome 2 location 60725424 to 60725688 (DHS +55 functional region), or excludes the entire region at location 60722238 to 60722466 (DHS +58 functional region), or excludes the entire region at location 60718042 to 60718186 (DHS +62 functional region), or excludes the entire region at location 60773106 to 60773435 (exon 2) of the human chromosome 2 is that according to UCSC Genome Browser hg 19 human genome assembly.

12) The RNP complex of any preceding paragraph, wherein the base editor protein is a third generation base editor.

13) The RNP complex of any preceding paragraph, wherein the base editor protein is A3A (N57Q)-BE3, A3A-BE3, or A3A (N57G)-BE3.

14) The RNP complex of any preceding paragraph for use in the ex vivo targeted genome editing of at least one target in a progenitor cell selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

15) The RNP complex of any preceding paragraph for use in an ex vivo method of producing a progenitor cell or a population of progenitor cells wherein the cells or the differentiated progeny therefrom have decreased BCL11A mRNA or protein expression.

16) The RNP complex of any preceding paragraph for use in an ex vivo method of producing an isolated genetic engineered human cell or a population of genetic engineered human cells having at least one genetic modification.

17) The RNP complex of any preceding paragraph for use in an ex vivo method of increasing fetal hemoglobin levels in a cell or in a mammal.

18) The RNP complex of any preceding paragraph, wherein the RNP complex is used in the electroporation of cells.

19) The RNP complex of any preceding paragraph, wherein the isolated progenitor cell or isolated cell is a hematopoietic progenitor cell or a hematopoietic stem cell.

20) The RNP complex of any preceding paragraph, wherein the hematopoietic progenitor is a cell of the erythroid lineage.

21) The RNP complex of any preceding paragraph, wherein the isolated progenitor cell or isolated cell is an induced pluripotent stem cell.

22) The RNP complex of any preceding paragraph, wherein the progenitor cell or human cell acquires at least one genetic modification.

23) The RNP complex of any preceding paragraph, wherein the at least one genetic modification is a deletion, insertion or substitution of the genetic sequence of the cell.

24) The RNP complex of any preceding paragraph, wherein the at least one genetic modification is a C to T, G, or A substitution.

25) The RNP complex of any preceding paragraph, wherein the at least one genetic modification is any base substitution.

26) The RNP complex of any preceding paragraph, wherein the at least one genetic modification is located between chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) of the human chromosome 2 is that according to UCSC Genome Browser hg 19 human genome assembly.

27) The RNP complex of any preceding paragraph, wherein the at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

28) The RNP complex of any preceding paragraph, wherein the at least one genetic modification results in the increase in fetal hemoglobin induction.

29) The RNP complex of any preceding paragraph, for use in treating beta-hemoglobin disorders selected from the group consisting of: sickle cell disease and beta-thalassemia.

30) A ribonucleoprotein (RNP) complex comprising:

a. a base editor protein; and

b. at least two nucleic acid sequences shown in Table 1, SEQ ID NOS: 1-139, wherein there is at least one chemical modification to a nucleotide in the nucleic acid sequence. 31) A nucleoprotein (RNP) complex comprising:

a. a base editor protein; and

b. at least one guide RNAs targeting an enhancer region and at least one guide RNA targeting an additional sequence.

32) The RNP complex of any preceding paragraph, wherein the enhancer region is a BCL11A enhancer region.

33) The RNP complex any preceding paragraph, wherein the at least one additional sequence is selected from the group selected from: gamma-globin promoter mutant sequence associated with HPFH, sickle cell HbS mutant sequence, sickle cell HbC mutant sequence, sickle cell HbD mutant sequence, β-Thalassemia HbE mutant sequence, and alpha-globin sequences

34) The RNP complex of any preceding paragraph, for use in correcting the β-Thalassemia HBB-28 A to G mutation.

35) A nucleoprotein (RNP) complex comprising:

a. a base editor protein; and

b. at least one guide RNAs targeting an enhancer region and at least one guide RNA targeting a promoter sequence.

36) The RNP complex of any preceding paragraph, wherein the enhancer region is a BCL11A enhancer region.

37) The RNP complex of any preceding paragraph, wherein the enhancer region is a BCL11A enhancer region is a +55 enhancer, +58 enhancer, or +62 enhancer.

38) The RNP complex of any preceding paragraph, wherein the promoter site is a HBG½ promoter site.

39) The RNP complex of any preceding paragraph, wherein the HBG½ promoter site is −115 or −198.

40) A ribonucleoprotein (RNP) complex comprising:

a. A3A (N57Q)-BE3; and

b. a nucleic acid sequences having the sequence of SEQ IN: 42, wherein there is at least one chemical modification to a nucleotide in the nucleic acid sequence.

41) A composition comprising a RNP complex of any preceding paragraph.

42) The composition of any preceding paragraph for use in the ex vivo targeted genome editing at least one target in a progenitor cell selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

43) The composition of any preceding paragraph for use in an ex vivo method of producing a progenitor cell or a population of progenitor cells wherein the cells or the differentiated progeny therefrom have decreased BCL11A mRNA or protein expression.

44) The composition of any preceding paragraph for use in an ex vivo method of producing an isolated genetic engineered human cell or a population of progenitor cells having at least one genetic modification.

45) The composition of any preceding paragraph for use in an ex vivo method for increasing fetal hemoglobin levels in a cell or in a mammal.

46) The composition of any preceding paragraph, wherein the composition is used in the electroporation of cells.

47) The composition of any preceding paragraph, wherein the step of electroporation is performed in a solution comprising glycerol.

48) A method for producing a progenitor cell or a population of progenitor cells having decreased BCL11A mRNA or protein expression, the method comprising contacting an isolated progenitor cell with an effective amount of an RNP complex of any preceding paragraph, or a composition of any preceding paragraph, whereby the contacted cells or the differentiated progeny cells therefrom have decreased BCL11A mRNA or protein expression.

49) The method of any preceding paragraph, wherein the contacted progenitor cell acquires at least one genetic modification in the human chromosome 2 is that according to UCSC Genome Browser hg 19 human genome assembly.

50) The method of any preceding paragraph, wherein the at least one genetic modification is a deletion, insertion or substitution of the genetic sequence of the cell.

51) The method of any preceding paragraph, wherein the at least one genetic modification is a C to T, G, or A substitution.

52) The method of any preceding paragraph, wherein the at least one genetic modification is located between chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) of the human chromosome 2 is that according to UCSC Genome Browser hg 19 human genome assembly.

53) The method of any preceding paragraph, wherein the at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198.

54) The method of any preceding paragraph, wherein the contacted cells or the differentiated progeny cells therefrom further have increased fetal hemoglobin levels.

55) A method for producing an isolated genetic engineered human cell or a population of genetic engineered isolated human cells having at least one genetic modification, the method comprising contacting an isolated cell or a population of cells with an effective amount of an RNP complex of any preceding paragraph, or a composition of any preceding paragraph wherein the at least one genetic modification produced is located in human chromosome 2 is that according to UCSC Genome Browser hg 19 human genome assembly.

56) A method of increasing fetal hemoglobin levels in a cell, the method comprising the steps of: contacting an isolated cell with an effective amount of a composition of an RNP complex of any preceding paragraph, or a composition of any preceding paragraph, thereby causing at least one genetic modification at the human chromosome 2 is that according to UCSC Genome Browser hg 19 human genome assembly therein, whereby fetal hemoglobin expression is increased in said cell, or its progeny, relative to said cell prior to said contacting.

57) The method of any preceding paragraph, wherein the isolated progenitor cell or isolated cell is a hematopoietic progenitor cell or a hematopoietic stem cell.

58) The method of any preceding paragraph, wherein the hematopoietic progenitor is a cell of the erythroid lineage.

59) The method of any preceding paragraph, wherein the isolated progenitor cell or isolated cell is an induced pluripotent stem cell.

60) The method of any preceding paragraph, wherein the isolated progenitor cell or isolated cell is contacted ex vivo or in vitro.

61) The method of any preceding paragraph, wherein the contacted progenitor cell or contacted cell acquires at least one genetic modification.

62) The method of any preceding paragraph, wherein the at least one genetic modification is a deletion, insertion or substitution of the nucleic acid sequence of chromosome 2.

63) The method of any preceding paragraph, wherein the at least one genetic modification is a C to T, G, or A substitution.

64) The method of any preceding paragraph, wherein the at least one genetic modification is any base substitution

65) The method of any preceding paragraph, wherein the at least one genetic modification is located between chromosome 2 location 60725424 to 60725688 (+55 functional region), at location 60722238 to 60722466 (+58 functional region), or at location 60718042 to 60718186 (+62 functional region), or at location 60773106 to 60773435 (exon 2).

66) The method of any preceding paragraph, wherein the contacted progenitor cell or contacted cell are further electroporated.

67) The method of any preceding paragraph, wherein the step of electroporation is performed in a solution comprising glycerol.

68) An isolated genetic engineered human cell or a population of genetic engineered human cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) according to any preceding paragraph.

69) An isolated genetic engineered human cell or a population of genetic engineered human cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198 according to any preceding paragraph.

70) A population of genetically edited progenitor cells having at least one genetic modification on chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) according to any preceding paragraph.

71) A population of genetically edited progenitor cells having at least one genetic modification is located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198 according to any preceding paragraph.

72) A composition comprising isolated genetic edited human cells of any preceding paragraph.

73) A composition comprising the population of genetically edited progenitor cells of any preceding paragraph.

74) A method for increasing fetal hemoglobin levels in a mammal in need thereof, the method comprising the steps of:

(a) ex vivo contacting an isolated hematopoietic progenitor cell isolated from said mammal with an effective amount of an RNP complex of any preceding paragraph, or a composition of any preceding paragraph, whereby the base editor protein targets the genomic DNA of the cell, causing at least one genetic modification therein, whereby fetal hemoglobin expression is increased in said cell or the differentiated progeny cells therefrom, relative to expression prior to said contacting, and

(b) transplanting the contacted cells of (a) or culture expanded cells therefrom into said mammal.

75) A method for increasing fetal hemoglobin levels in a mammal in need thereof, the method comprising transplanting into the mammal:

(a) an isolated genetic engineered human cell of any preceding paragraph, or

(b) a population of genetically edited progenitor cells any preceding paragraph, or

(c) a composition of any preceding paragraph, or

(d) the progeny cells from of (a) or (b).

### EXAMPLES

### Example 1

Base editing with fusions of RNA-guided DNA-binding proteins and nucleotide deaminases represents a promising approach to permanently remedy genetic blood disorders without obligatory double strand breaks, however its application in engrafting hematopoietic stem cells (HSCs) remains unexplored. Here we purified A3A(N57Q)-BE3 protein for RNP electroporation of human peripheral blood (PB) mobilized CD34+ hematopoietic stem and progenitor cells (HSPCs). We found that sgRNAs targeting for cytidine base editing the core GATA1 binding motif of the BCL11A +58 erythroid enhancer resulted in efficient on-target base edits (81% allele frequency) with low indels. There was similar HbF induction in erythroid progeny as compared to Cas9:sgRNA RNP nuclease mediated modification of the same target sequence (36% median HbF in base edited cells with 81% allele modifications, 39% HbF in 3xNLS-SpCas9:sgRNA#1617 nuclease edited cells with 99% indels, and 5% HbF in unedited cells). A single therapeutic base edit of the BCL11A enhancer was sufficient to ameliorate the pathobiology of both sickle cell disease (SCD) and beta-thalassemia. Base editing of CD34+ HSPCs from plerixafor mobilized PB SCD patient donors resulted in potent HbF induction (24-31% HbF level in bulk cells with 87-90% on-target allele modifications) and reduced sodium metabisulfite induced sickling (from 84% to 29% of erythrocytes). Base editing of non-mobilized PB CD34+ HSPCs from 3 beta-thalassemia patient donors potently induced gamma-globin and improved ineffective erythropoiesis in vitro, with 59-75% on-target allele modifications. Moreover highly efficient multiplex editing could be achieved in HSPCs. Simultaneous disruption of the BCL11A +58 erythroid enhancer core GATA1 motif and correction of the HBB -28A>G promoter mutation in HSPCs resulted in production of at least one therapeutic allele in 35 of 35 erythroid colonies analyzed, with each colony achieving improved alpha-to-non-alpha globin chain balance. Finally we found that base editing could be efficiently produced in engrafting HSCs. Using 3 donors, we observed 62-64% base edits at the BCL11A +58 enhancer GATA1 motif in input HSPCs with one round of electroporation and 73-85% base edits with two rounds of electroporation (separated by 24 hours), albeit with modest reduction of cell viability with tandem electroporation. We observed 70-94% human chimerism and 35-82% base edits in engrafting human cells in primary recipients and 11-61% base edits in secondary recipients as measured 16 weeks following cell infusion. Similar edit frequencies were observed across all assayed hematopoietic lineages including engrafting B-lymphocytes, erythroid precursors, and HSPCs. We observed that compared to non-engrafting progenitors, long-term engrafting HSCs favored C to T (as compared to C to G/A) editing. There was a strong correlation (Spearman r 0.86, P<0.0001) between base edit frequency and HbF level of engrafting erythroid cells, with in vivo HbF level induced from median 1.8% in unedited recipients to 22% in recipients of tandem base edited cells. Together these results demonstrate, for the first time, the potential of RNP base editing of human HSPCs as an alternative to nuclease editing for HSC-targeted therapeutic genome modification.

### Example 2

### Introduction

Base editing by nucleotide deaminases linked to programmable DNA-binding proteins represents a promising approach to permanently remedy blood disorders, although its application in engrafting hematopoietic stem cells (HSCs) remains unexplored. Here we purified A3A (N57Q)-BE3 protein for ribonucleoprotein (RNP) electroporation of human peripheral blood (PB) mobilized CD34⁺ hematopoietic stem and progenitor cells (HSPCs). We found that sgRNAs targeting for cytosine base editing the core GATA1 binding motif of the BCL11A +58 erythroid enhancer resulted in efficient on-target base edits with low indel frequency. There was similar fetal hemoglobin (HbF) induction in erythroid progeny after base editing as compared to Cas9:sgRNA RNP nuclease mediated disruption of the same target sequence. A single therapeutic base edit of the BCL11A enhancer was sufficient to prevent sickling and ameliorate globin chain imbalance in erythroid progeny from sickle cell disease (SCD) and β-thalassemia patient derived HSPCs respectively. Moreover highly efficient multiplex editing could be achieved in HSPCs with combined disruption of the BCL11A erythroid enhancer and correction of the HBB -28A>G promoter mutation. Finally we found that base edits could be efficiently produced in multilineage-repopulating self-renewing human HSCs as assayed in primary and secondary recipient animals. Compared to non-engrafting progenitor cells, quiescent HSCs favored C to T editing. Base editing of the BCL11A enhancer resulted in potent HbF induction in vivo. Together these results demonstrate, to our knowledge for the first time, the potential of RNP base editing of human HSPCs as a feasible alternative to nuclease editing for HSC-targeted therapeutic genome modification.

### Results

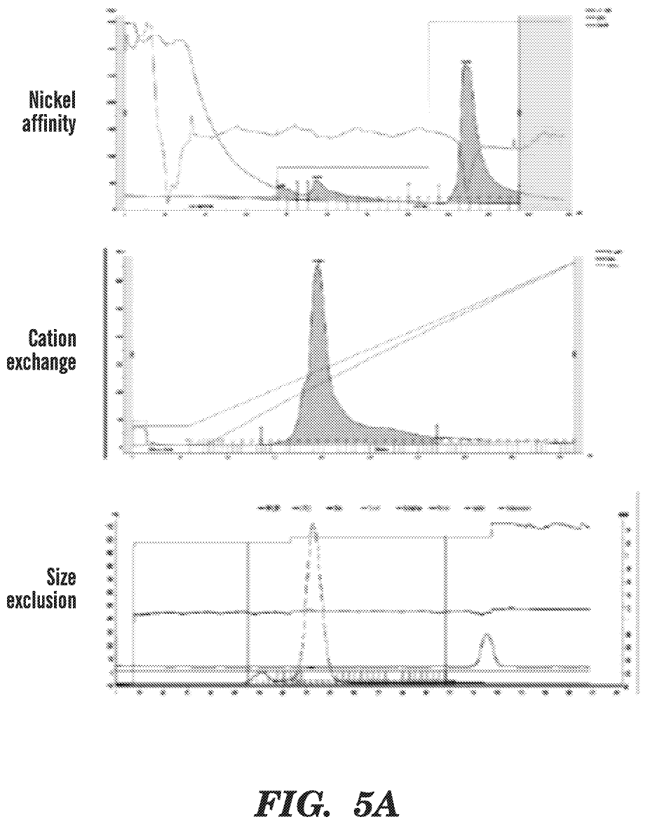

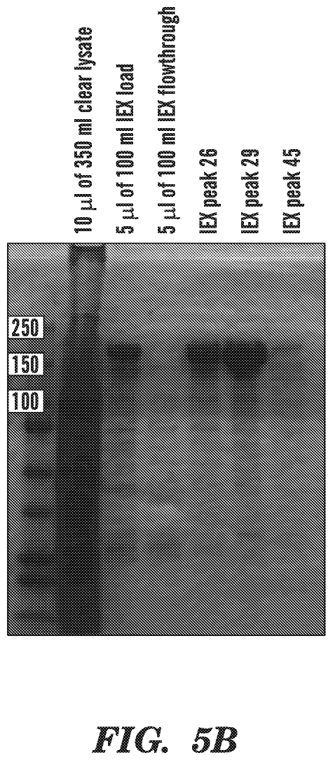

Delivery to HSCs of programmable endonucleases such as Cas9:sgRNA RNP complexes can lead to highly efficient genome editing, especially as mediated by non-homologous end joining (NHEJ) repair, that could plausibly contribute to cure of blood disorders1-7. For example, biallelic indels within a GATA1 binding motif at the core of the +58 BCL11A erythroid enhancer yield efficient motif disruption, selective reduction of BCL11A expression in erythroid cells, preserved HSC function, and potent HbF induction in SCD and β-thalassemia erythroid progeny8. In contrast to endonuclease genome editing, base editing does not rely on double-strand breaks (DSBs) but rather leverages programmable single base pair conversion9-12. The feasibility of base editing in HSCs to enable durable therapeutic modification of blood cells remains uncertain. The A3A (N57Q)-BE3 base editor is a recently developed base editor protein that targets cytosine within TCR motif context while reducing bystander mutations in the base editing window by replacing rat APOBEC1 (rAPO1) in the third generation base editor (BE3) with an engineered human APOBEC3A (A3A) domain13. We found that we could recover this base editor protein with high purity and yield (FIGS. 5a and 5b).

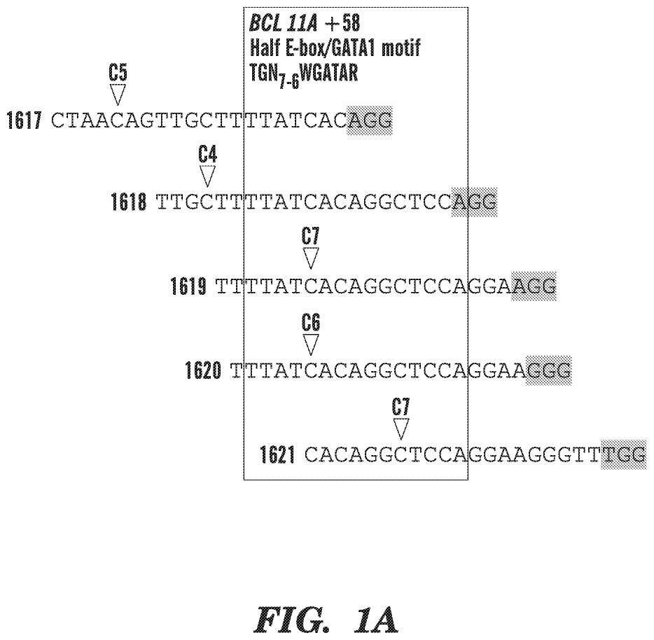

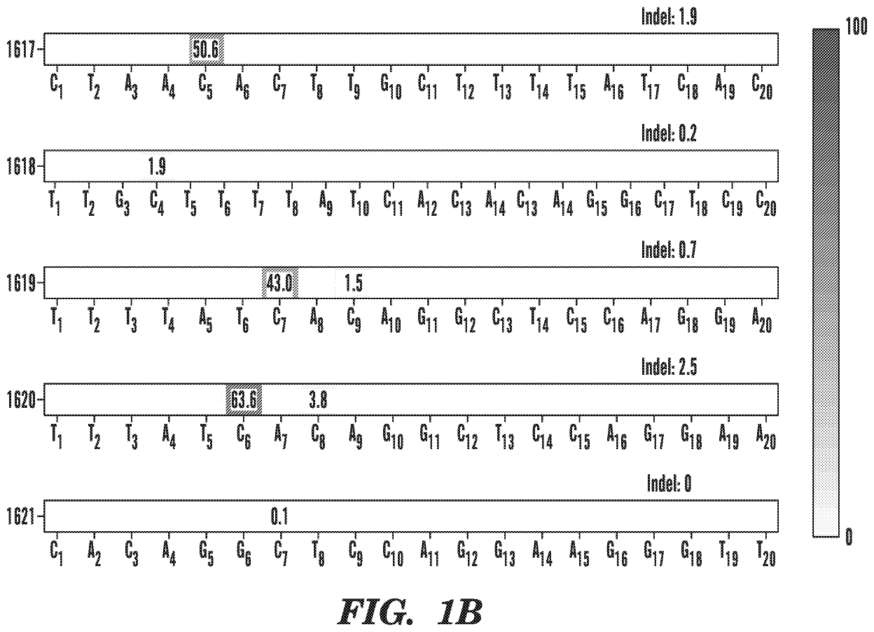

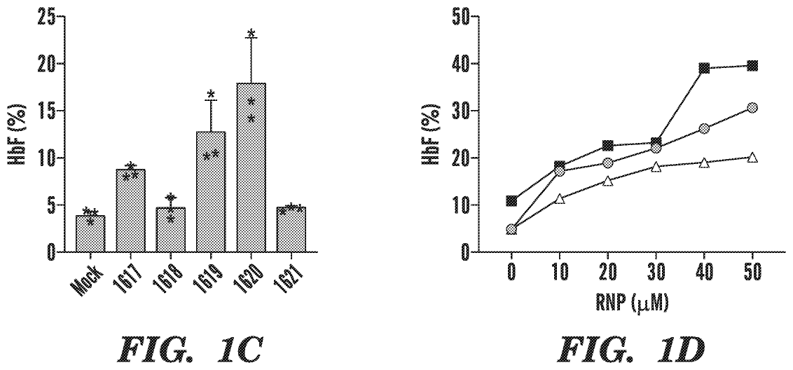

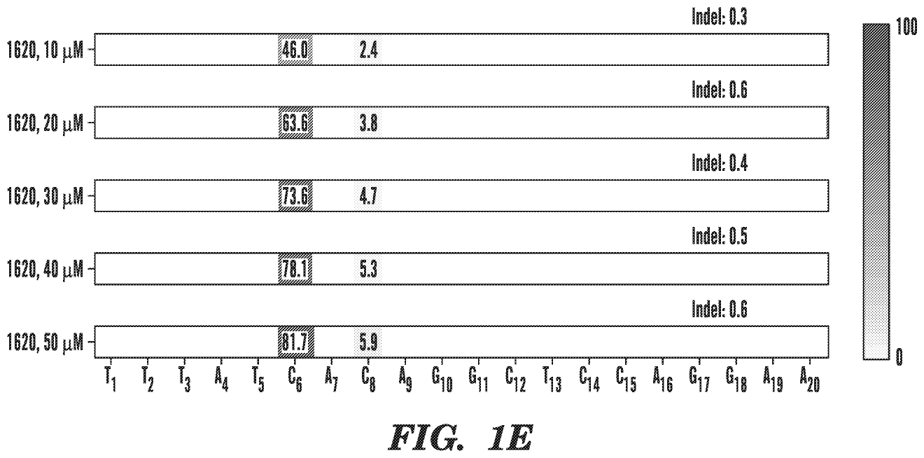

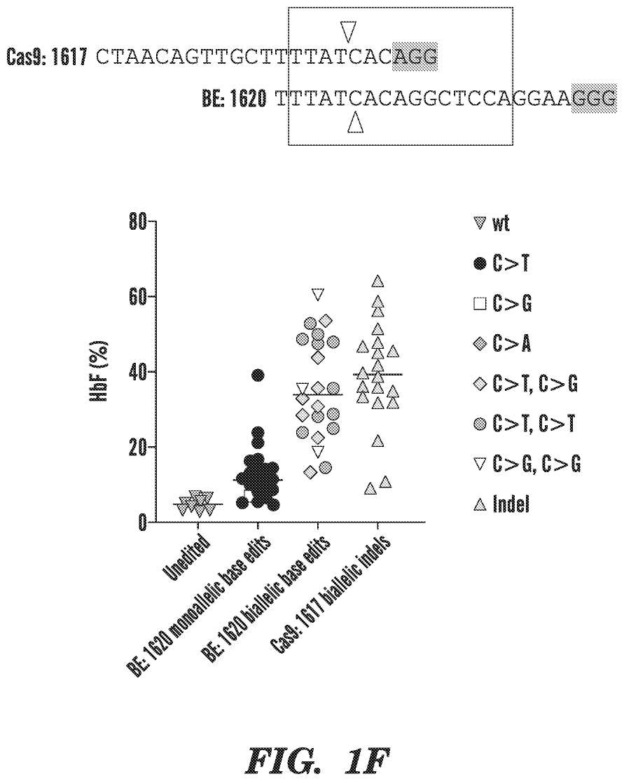

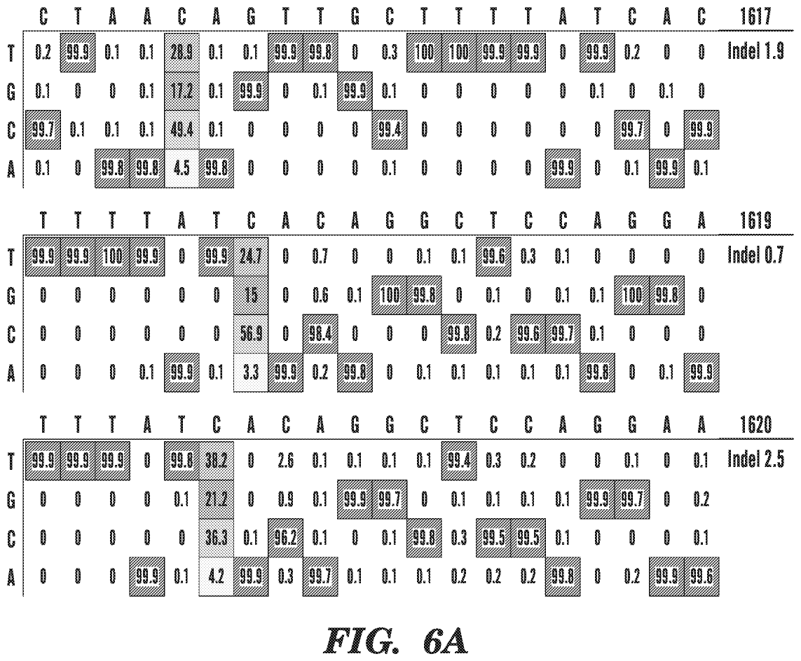

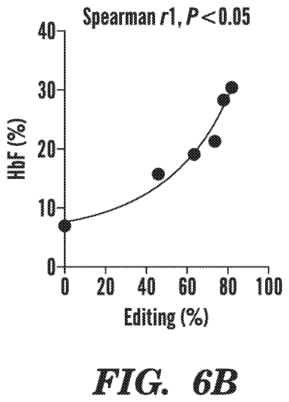

We electroporated A3A (N57Q)-BE3 base editor with chemically modified synthetic sgRNAs as ribonucleoprotein (RNP) complexes targeting the core of the +58 BCL11A erythroid enhancer in human CD34+ HSPCs. At the target locus, five potential sgRNAs include cytosine at protospacer position 5-9. Three of them have the target cytosine within a core TGN7-9WGATAR half E-box/GATA binding motif bound by the erythroid transcription factors GATA1 and TAL1 (FIG. 1a)8,14,15. Editing with sgRNA-1620 directly converted C at protospacer position 6 to T, G, and A at frequencies of 38.2%, 21.2%, and 4.2% respectively (FIG. 6a). Even though the protospacer sequences targeted by sgRNA-1619 and sgRNA-1620 are only shifted by one base pair, base edit efficiencies were lower for the target C at position 7 when editing with sgRNA-1619 (43.0% at C7) compared to sgRNA-1620 (63.6% at C6, FIG. 1b and FIG. 6a). While sgRNA-1617 yielded 50.6% base editing at C5, this site was not within the half E-box/GATA binding motif (FIG. 1a and FIG. 6a). Consistent with the highest mutation frequencies at the core GATA1 binding sequence, editing with sgRNA-1620 resulted in the highest HbF level in erythroid progeny (FIGS. 1b and 1c). We electroporated human CD34+ HSPCs with sgRNA-1620 complexed with A3A (N57Q)-BE3 base editor at different concentrations ranging from 10-50 μM. Base editing was dose-dependent, with 50 μM RNP producing 81.7% base edits at position C6. There was a strong correlation (Spearman r 1, p<0.05) between base edit frequency and HbF level (FIGS. 1d, 1e, FIG. 6b). We subjected HSPCs to single cell clonal erythroid liquid culture following base editing to compare genotype to globin expression. For comparison we also performed highly efficient nuclease editing using 3xNLS-SpCas9:sgRNA-1617 RNP8. The sgRNA-1620 base editing site and sgRNA-1617 indel site lead to modifications at the same GATA1 motif within their respective editing windows (FIG. 1f). Although monoallelic base editing was insufficient for robust HbF induction, biallelic base editing by either C>T or C>G resulted in similar HbF induction as biallelic indels (FIG. 1f). These results suggest that substitution of a single nucleotide within the core BCL11A +58 enhancer GATA1 motif is sufficient for robust HbF induction.

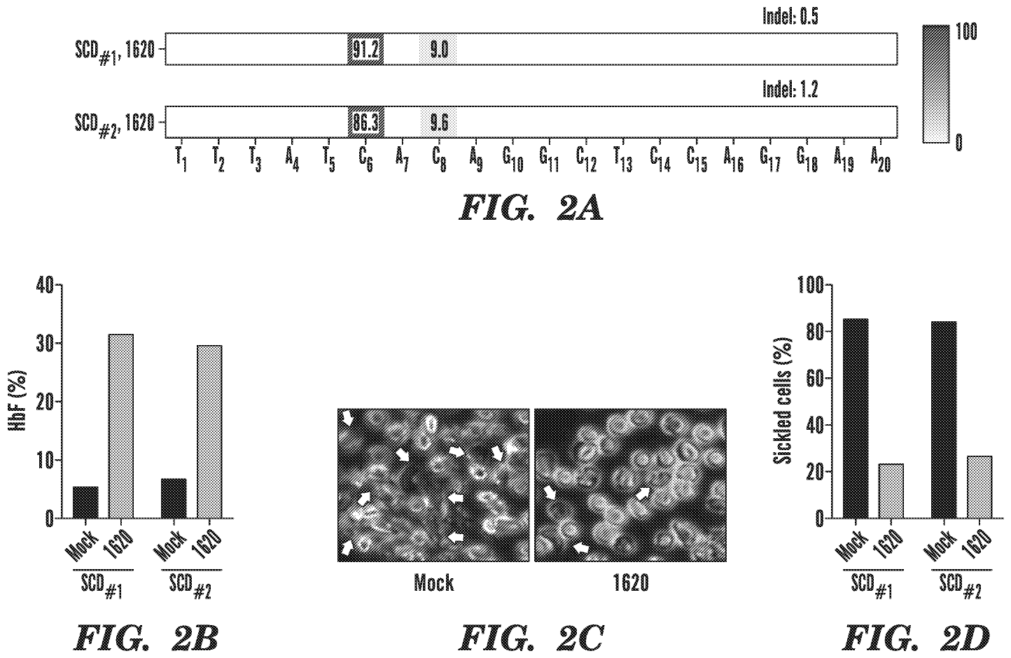

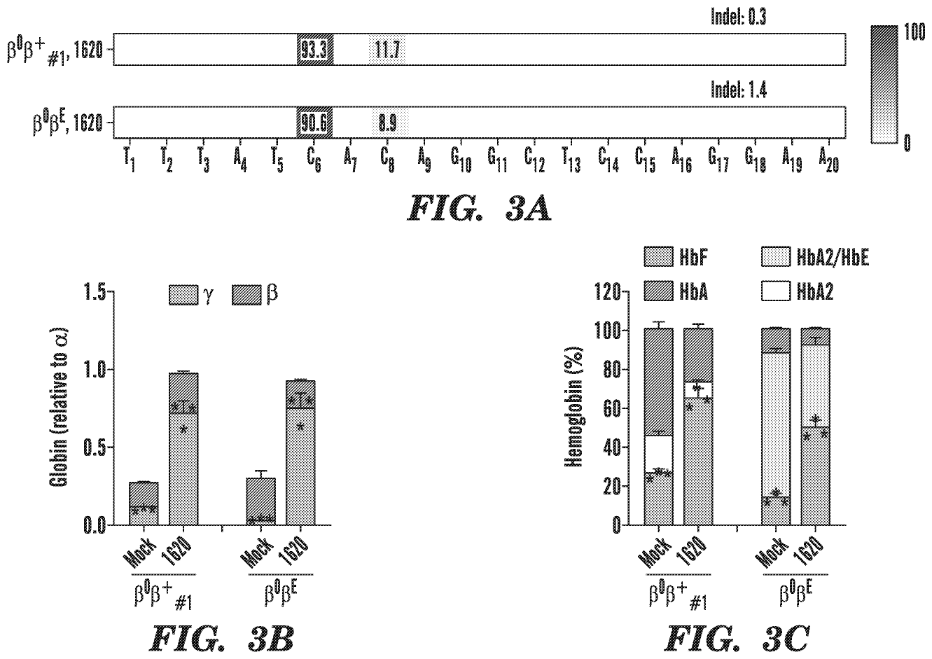

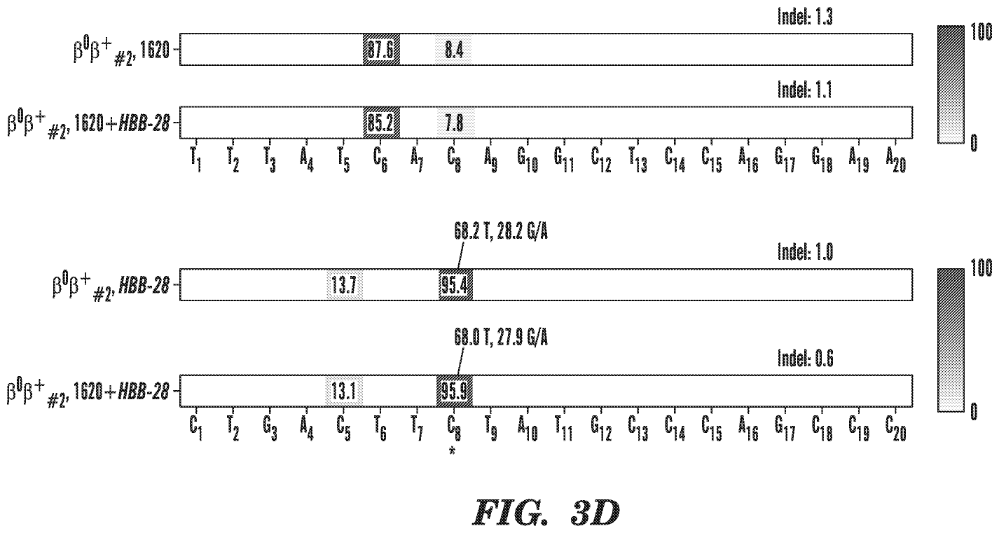

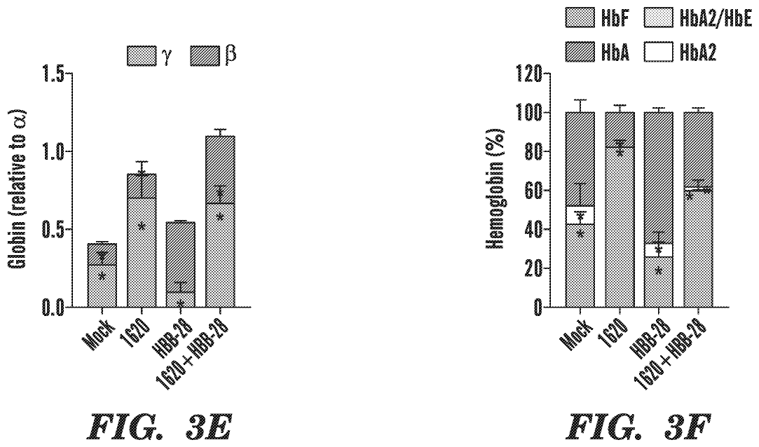

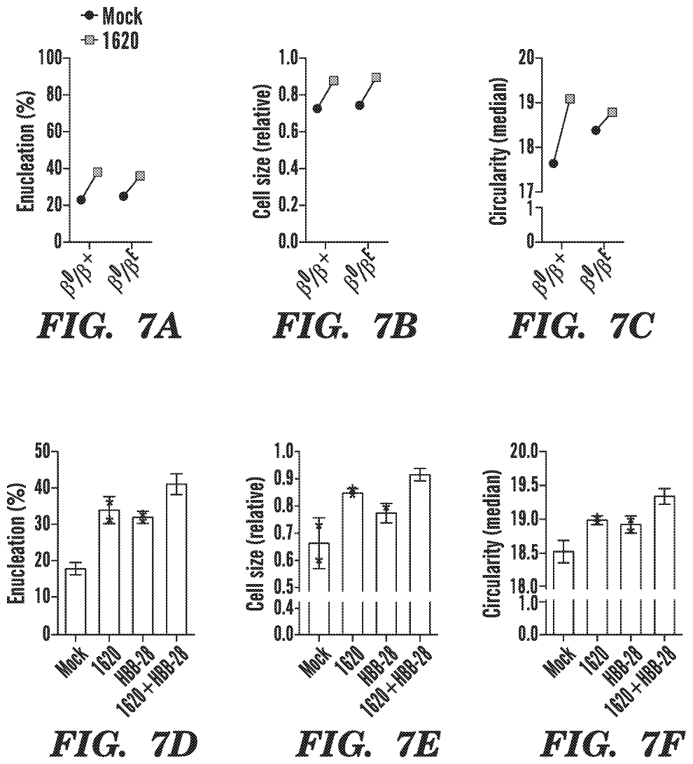

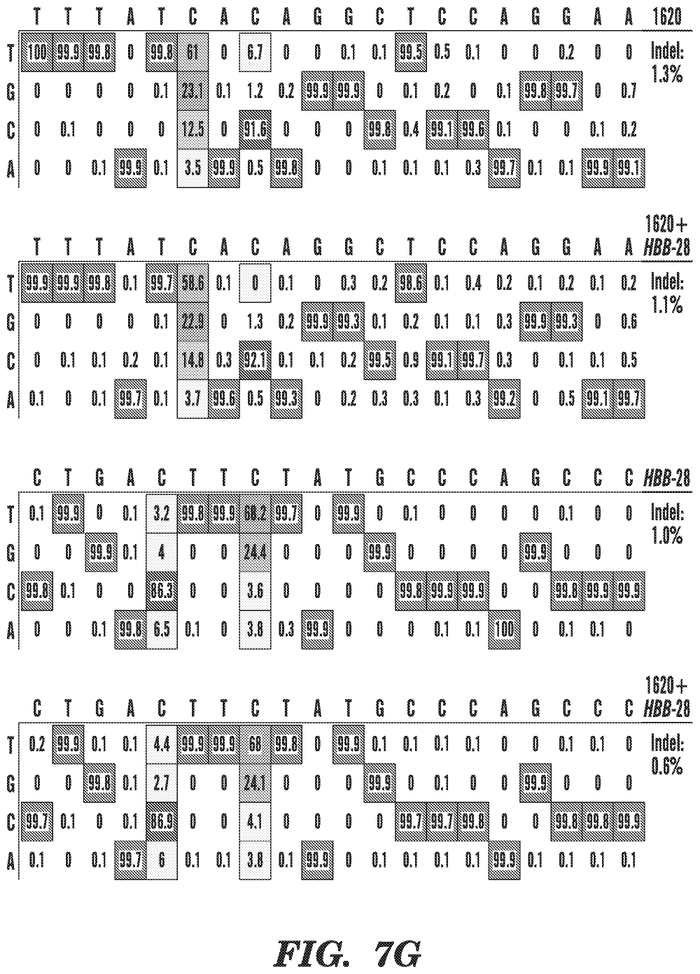

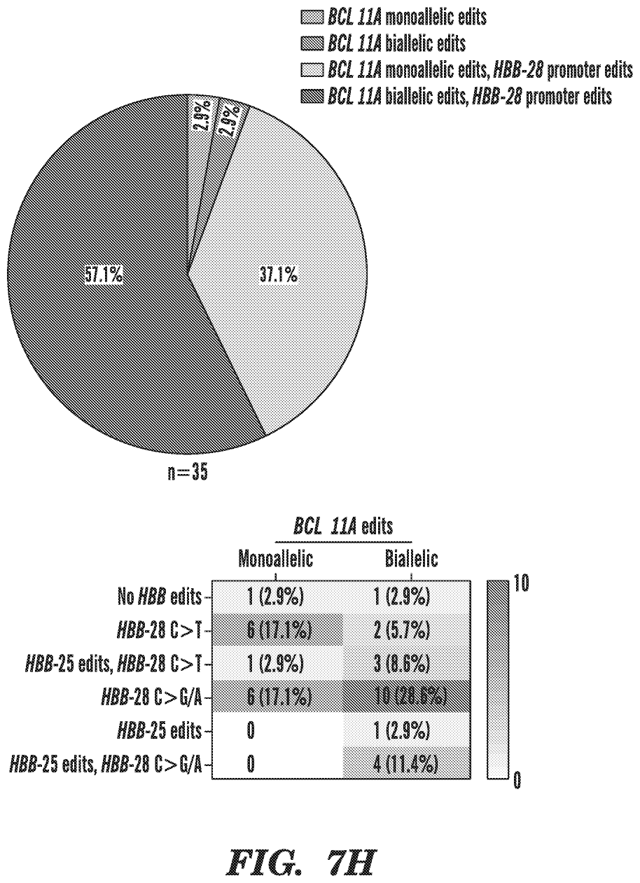

To further examine the potential benefit of base editing with regard to disease pathobiology, we evaluated therapeutic HbF induction by A3A (N57Q)-BE3 RNP editing of primary HSPCs from SCD and β-thalassemia patients. We performed two cycles of RNP electroporation separated by 24 hours to maximize base editing efficiency. Using plerixafor-mobilized peripheral blood CD34+ HSPCs from two SCD donors, we observed 91.2% and 86.3% editing at C6 within the targeting window (FIG. 2a). The bulk base edited erythroid progeny demonstrated 31.3% and 29.3% HbF (FIG. 2b). While unedited enucleated erythroid cells showed robust sickling by sodium metabisulfite (MBS) treatment, we observed substantially fewer sickled cells following base editing (p<0.05, FIGS. 2c and 2d). In addition, we edited non-mobilized peripheral blood CD34+ HSPCs from two β-thalassemia patients (one each with 13013+ and β0βE genotype). Base edit frequencies at the target BCL11A enhancer cytosine following A3A (N57Q)-BE3 RNP:sgRNA-1620 electroporation were 93.3% and 90.6% (FIG. 3a). In each donor, base edited erythroid cells showed potent γ-globin and HbF induction (FIGS. 3b and 3c). Following base editing, we observed higher enucleation efficiency, larger size and more circular shape of enucleated erythroid cells from β-thalassemia donors, consistent with improved erythropoiesis (FIG. 7a-7c).

Previously we observed that in committed erythroid precursors A3A (N57Q)-BE3:sgRNA-HBB-28 RNP electroporation could selectively repair the common Chinese β-thalassemia promoter mutation HBB -28 A>G (T>C on complementary strand) while minimizing unwanted bystander cytosine modifications13. In fact, C>G/A/T at HBB promoter position -25 (C5 within the editing window) or C>G/A at -28 (C8 with respect to base editing window) could actually create de novo β-thalassemia alleles16-18. We evaluated A3A (N57Q)-BE3:sgRNA-HBB-28 RNP base editing by electroporation of HSPCs from a β-thalassemia patient compound heterozygous for the HBB -28 A>Gβ+ mutation and a null β0 allele (β0β+#2). Despite 18.2% corrective C>T editing at C8 (68.2% T includes non-mutant T allele), we observed 28.2% non-corrective C>G/A edits at C8, 3.6% unedited C8 alleles, and 13.7% disruptive C5 base edits (FIGS. 3d and 7g). We speculated that multiplex base editing with A3A (N57Q)-BE3 targeting both the HBB -28 A>G mutation and BCL11A enhancer +58 GATA motif could produce positive interactive effects. Multiplex editing led to similar base edits at each target site as compared with single editing (FIG. 3d, FIG. 7g). Multiplex editing resulted in higher β-globin expression than sgRNA-1620 editing alone and higher β-globin expression than sgRNA-HBB-28 editing alone (FIG. 3e-3f). Multiplex edited erythroid cells showed increased enucleation frequency, cell size and circularity (FIG. 7d-7f). Colony analysis demonstrated the potentially complementary nature of combined base editing. For example, HBB-28 C>T editing restored β-globin expression in BCL11A enhancer monoallelic edited colonies. Reciprocally, biallelic BCL11A enhancer editing produced robust HbF induction in colonies in which HBB-28 editing failed to correct the thalassemia mutation (FIG. 7i). 27/35 (77.1%) of colonies had biallelic edits at BCL11A enhancer and/or corrective C>T editing at HBB promoter (FIG. 7h). These results indicate that RNP base editing can produce efficient therapeutically relevant multiplex edits in primary human HSPCs.

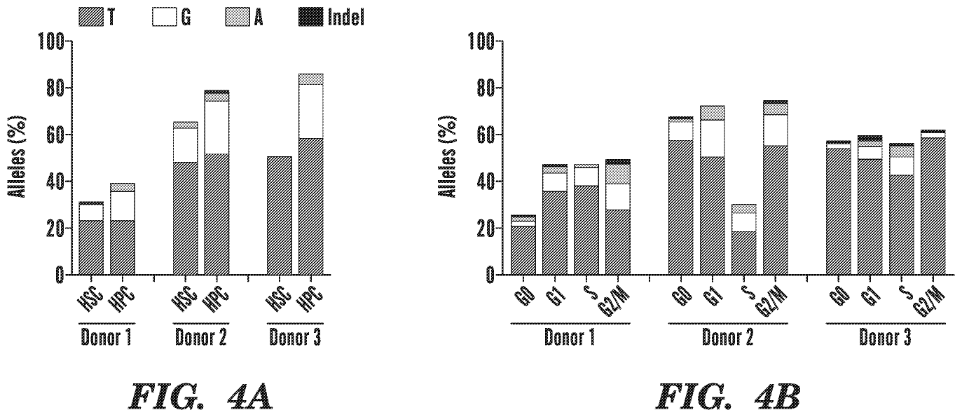

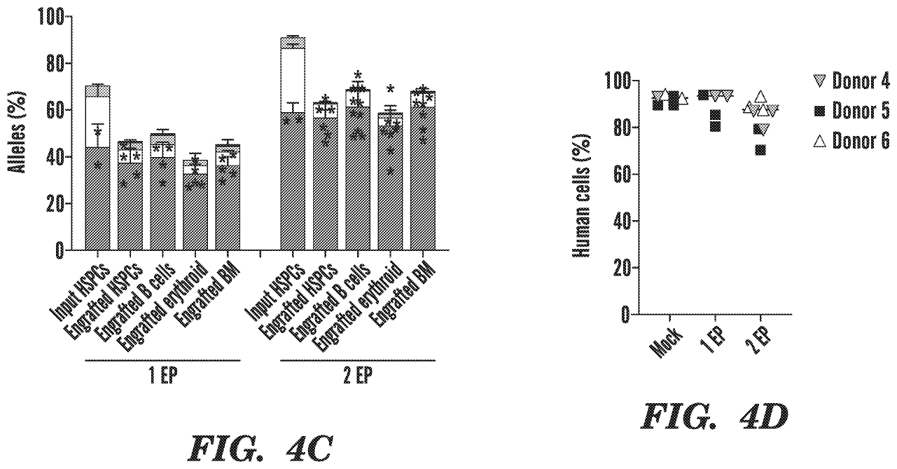

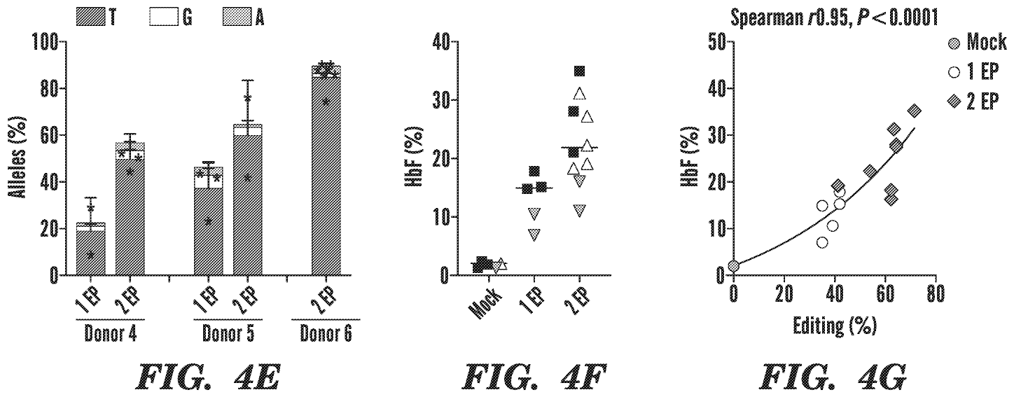

The types and frequencies of nuclease-mediated gene edits in engrafting HSCs often differ as compared to those in bulk HSPCs2,8,19. HSCs tend to be more refractory than progenitors to overall editing and favor NHEJ repair over resection-based homology-dependent recombination (HDR) and microhomology-mediated end-joining (MMEJ) repair modes. To investigate base editing in HSCs, we sorted an HSC-enriched CD34+ CD38− CD90+ CD45RA− population and a committed hematopoietic progenitor cell (HPC) enriched CD34+ CD38+ population two hours following A3A (N57Q)-BE3:1620 RNP electroporation of HSPCs from three healthy donors. Overall base editing frequencies in HSC enriched cells were lower than in HPCs (FIG. 4a). Most of this reduction was due to lower C>G/A frequencies in HSC enriched cells as compared to HPCs (FIG. 4a). In contrast, C>T frequencies were similar between HSC and HPC populations. Since HSCs tend to be quiescent relative to HPCs, we sorted G0, G1, S and G2/M phase HSPCs to evaluate potential cell cycle dependence of base editing. We found that C>G/A frequencies were reduced in G0 phase HPSCs compared with G1, S and G2/M phase HSPCs (FIG. 4b). These results suggest that base editing of quiescent HSCs may be more challenging than proliferative HPCs, similar to observations of nuclease-based hematopoietic gene editing.

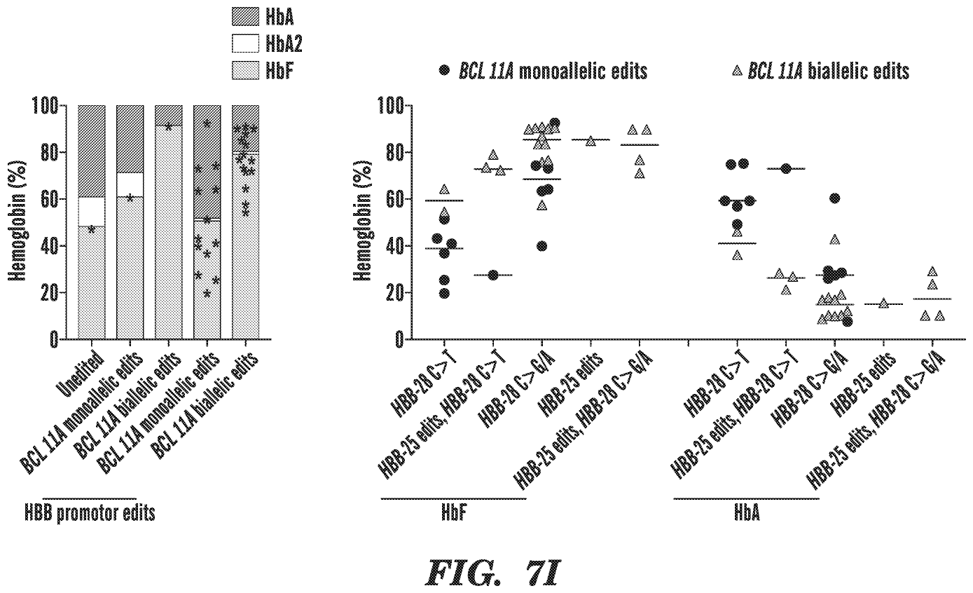

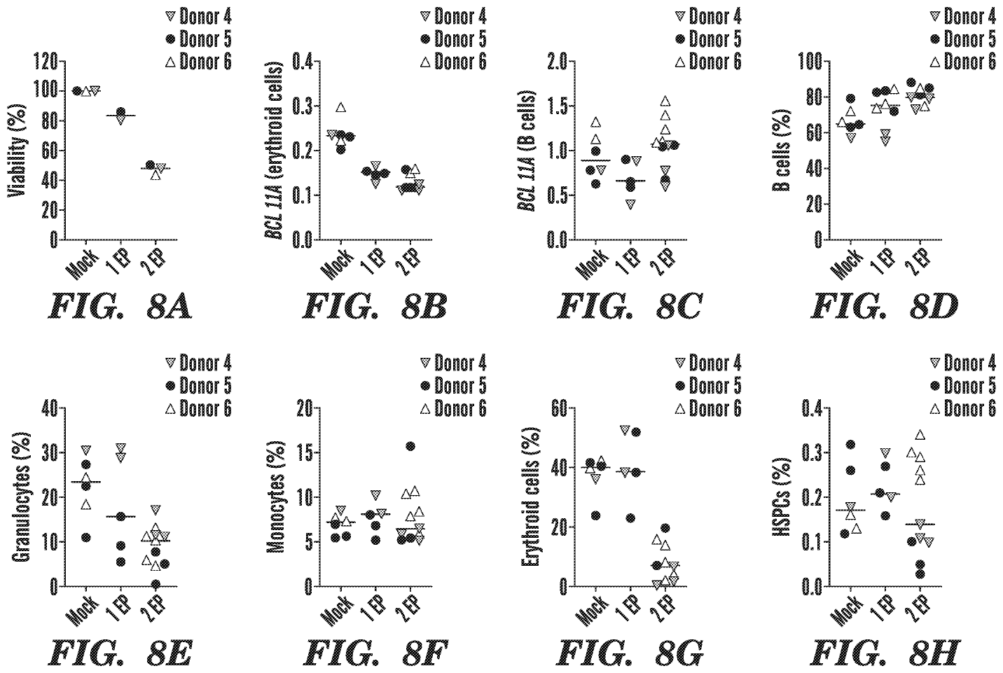

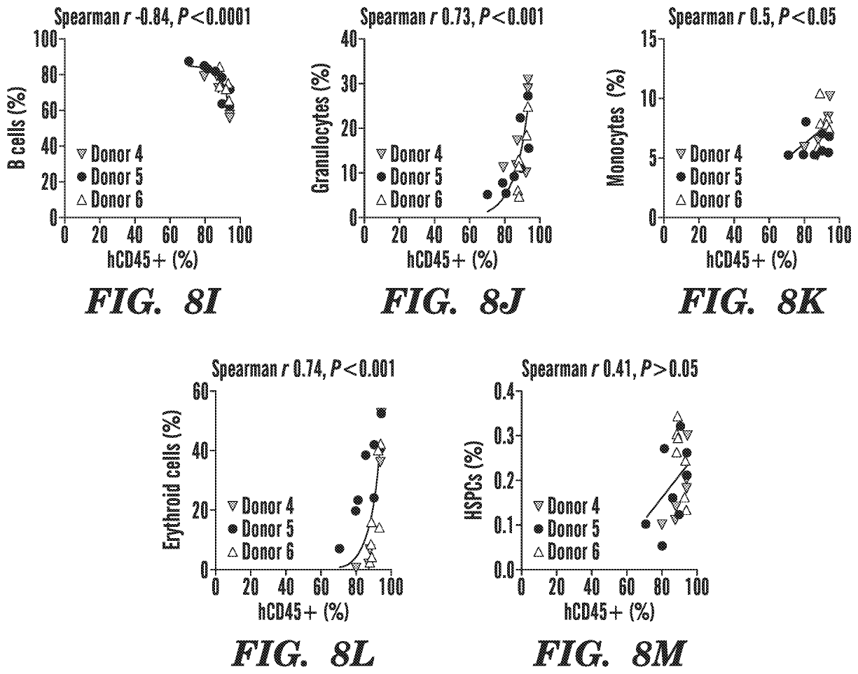

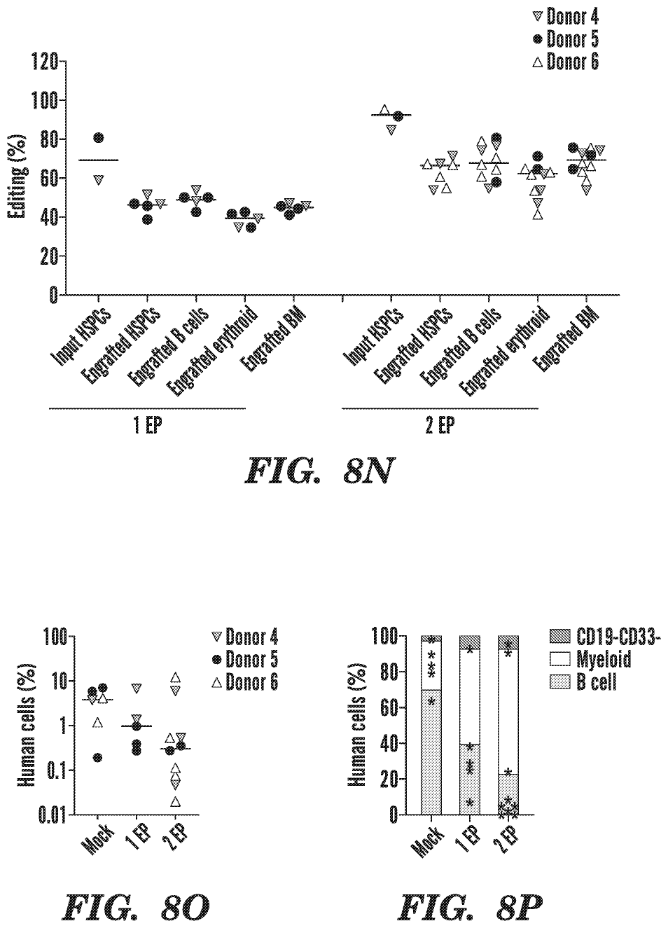

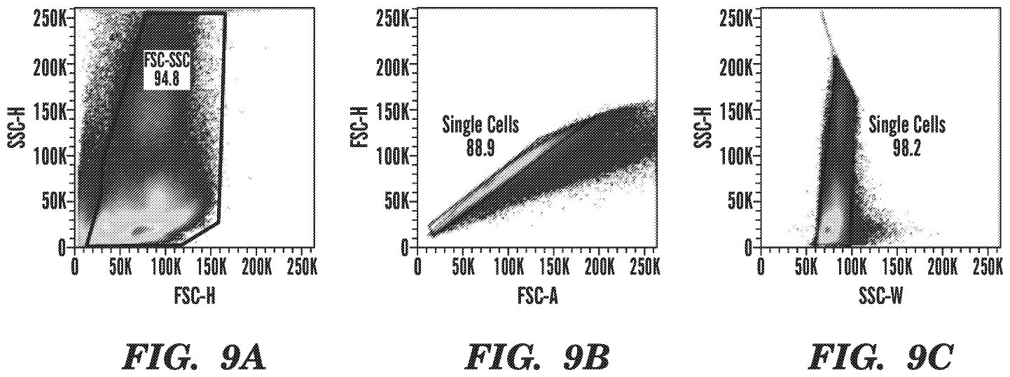

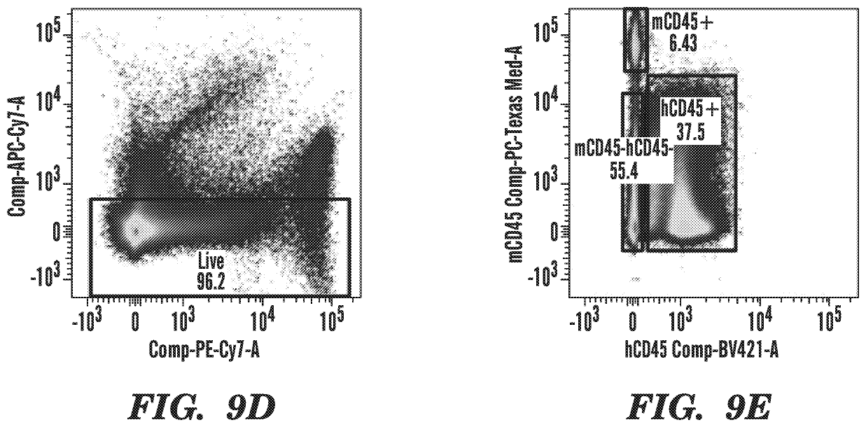

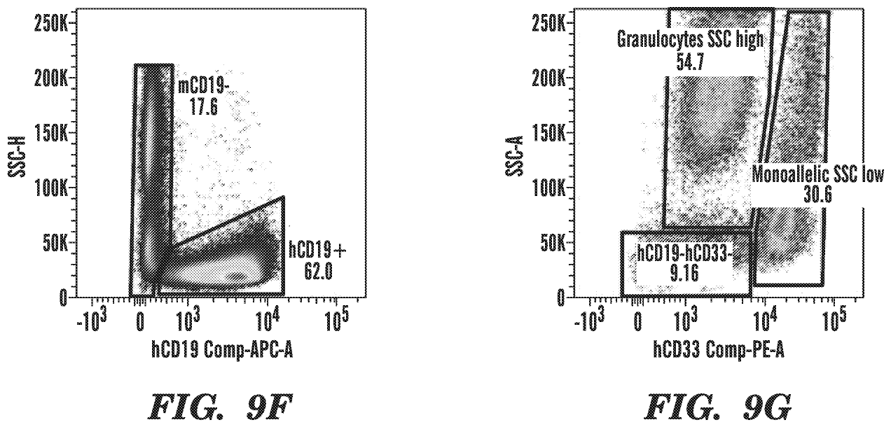

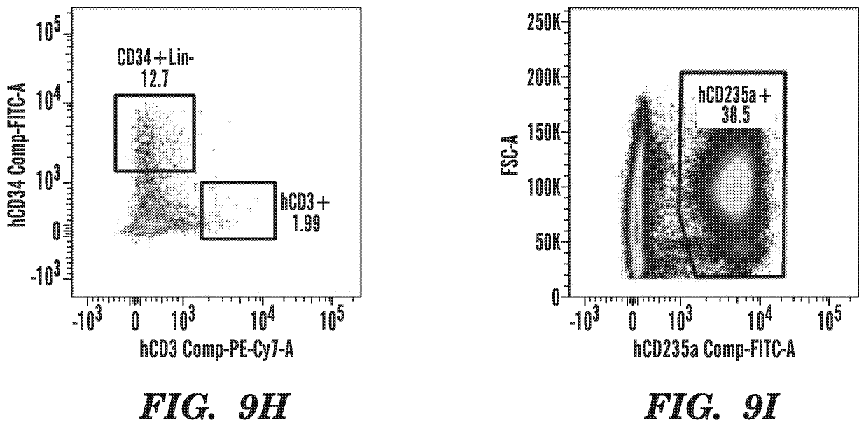

We attempted to augment base editing frequency in HSCs by performing a second cycle of RNP electroporation 24 hours after initial electroporation. We found that base editing frequency increased from median 70.3% with one cycle of electroporation to 92.5% with two cycles of electroporation, although the HSPC viability was reduced from mean 83% to 47% (FIG. 4c, FIGS. 8a and 8n).

To evaluate the functional potential of base edited HSCs, we infused edited CD34+ HSPCs from three healthy donors following one or two cycles of RNP electroporation into NBSGW mice, which support multilineage human hematopoietic engraftment in the absence of conditioning therapy20. After 16 weeks we measured human hematopoietic engraftment from isolated bone marrow. There was similar engraftment of HSPCs with one cycle of RNP electroporation compared to non-electroporated HPSCs (median 92.7% for mock and 93.5% for 1 EP cells, FIG. 4d). In contrast there was modest reduction in human engraftment with two cycles of RNP electroporation (87.5%, FIG. 4d).

The overall base editing frequency in engrafted BM following one cycle of RNP electroporation was 45.5% as compared to 70.3% in the input HSPCs. The overall base editing frequency in engrafted BM following two cycles of RNP electroporation was 69.1% compared with 92.5% in input HSPCs (FIG. 4c, FIG. 8n). Most of the reduction in base editing frequency in engrafting cells resulted from loss of C>G/A base edits. C>G/A edits comprised 26.5% and 32.1% of alleles in input HSPCs following one and two cycles of RNP electroporation but only 10.4% and 6.1% of alleles in engrafted BM (FIG. 4c). In contrast, the C>T base edited allele frequencies were similar comparing the input to engrafted cells, with 43.8% input and 35.1% engrafted with one cycle of RNP electroporation and 56.4% input and 63.4% engrafted with two cycles of RNP electroporation. These results are consistent with the ex vivo studies suggesting that engrafting quiescent HSCs as compared to non-engrafting proliferative progenitors favor C>T over C>G/A base edits.

The hallmark features of HSCs are the capacity for multilineage hematopoietic repopulation and self-renewal. To evaluate differentiation potential, we performed flow cytometry of bone marrow, staining for markers of lymphoid, myeloid and erythroid lineages as well as engrafting HSPCs. We observed multilineage engraftment in all recipients. Mice with greater human chimerism demonstrated more granulocyte and erythroid contribution with reciprocal reduction in B-lymphocyte contribution (Spearman r 0.73, 0.74, −0.84 and p<0.001, <0.001, <0.0001 for granulocyte, erythroid, and B-lymphoid contribution respectively, FIG. 8d-8m). We found similar base editing allele frequency in engrafting B-lymphoid cells, erythroid cells, and HSPCs (39.3-49.8% for 1 EP and 62.3-68.7% for 2 EP, FIG. 8n).

We performed secondary transplantation to evaluate the self-renewal potential of HSCs. There was multilineage human hematopoietic engraftment in each secondary recipient consistent with HSC activity (FIG. 8o-8p). We observed median 37% base edits in secondary BM recipients with one cycle of RNP electroporation and 79.5% base edits in secondary BM recipients with two cycles of RNP electroporation (FIG. 4e). These data suggest the feasibility to produce base edits in long-term HSCs.

Since NBSGW mice support erythroid repopulation with strong HbF repression, this is a stringent system to evaluate HbF induction potential8,21. After base editing, we found that BCL11A expression was decreased in engrafting erythroid cells while maintained in B-lymphoid cells, as expected for erythroid-specific enhancer disruption (FIG. 8b-8c). Median HbF levels were 1.8% in engrafting erythroid cells from unedited HSPCs, 14.7% after one cycle of RNP electroporation, and 21.7% after two cycles of RNP electroporation (FIG. 4f). There was a strong correlation between base edit frequency and HbF level of engrafting erythroid cells (Spearman r 0.95, p<0.0001, FIG. 4g), supporting the therapeutic potential of this single base edit.

### Discussion

Programmable endonuclease-mediated genome editing of hematopoietic cells is under active clinical investigation for a number of hematologic, malignant, and infectious indications, including BCL11A enhancer editing for β-hemoglobinopathies, although each of the completed22,23 and ongoing (NCT 03653247, 03432364, 03745287, 03655678, 02500849, 03399448) trials leverages NHEJ-mediated genetic disruption. The therapeutic application of HDR may be especially challenging given requirement for co-delivery of donor DNA sequences, intrinsic resistance of quiescent stem cells, and competing repair by NHEJ. For example, in sickle mutation correction by HDR, concurrent NHEJ repair leads to HBB gene disruption producing de novo severe β-thalassemia alleles3-5. In contrast, base editing offers the potential for precise single base substitution with high product purity while bypassing requirement for DSBs or extrachromosomal template12,24. However the application of base editing in HSCs has not been investigated. Although viral transduction is a possible means to deliver genome editing factors, RNP pulse delivery may be preferable to maximize on-target editing while limiting off-target potential25. Here we purified A3A (N57Q)-BE3 RNP for use in HSPCs. We found that higher concentrations of base editor as compared to Cas9 RNPs were required to achieve on-target editing8. Furthermore, multiple cycles of electroporation could increase on-target base editing but decreased HSPC viability and engraftment potential. These results suggest that enhanced delivery of base editors to HSCs could further improve their therapeutic promise.

Nonetheless we observed efficient base edits in HSPCs. We found that a single base edit at core sequences of the +58 BCL11A erythroid enhancer produced similar HbF induction as Cas9 nuclease mediated indels, resulted in therapeutically relevant HbF induction in erythroid cells derived from β-thalassemia and SCD patient HSPCs, and generated durable base edits in multilineage-repopulating self-renewing HSCs. An unexpected observation was that HSCs favored C>T as compared to C>G/A base edits, suggesting intrinsic preferences for DNA damage repair in HSCs, analogous to has been observed for DSB repair19.

One general concern for genome editing is off-target genotoxicity. Although guide RNA specific off-targets may be evaluated and mitigated by established methods extrapolated from nuclease-mediated off-target detection26, other off-targets of base editing may include guide RNA independent effects27,28, such as RNA and DNA editing. Pulse RNP delivery would not be expected to produce enduring RNA editing. The base editor we used has an attenuated cytosine deaminase domain A3A (N57Q)13. Similarly attenuated A3A-BE3 editors have been shown to substantially reduce RNA off-targets29. The impact of attenuated deaminases on guide-independent DNA off-target potential requires future investigation.

In summary, these studies demonstrate that highly efficient, specific, and disease ameliorating base editing in human HSCs is feasible with RNP delivery and may encourage therapeutic application of base editing for a range of disorders in which corrected or augmented hematopoiesis could be beneficial.

### Methods and Materials

Human CD34⁺ HSPCs from mobilized peripheral blood of deidentified healthy donors were obtained from Fred Hutchinson Cancer Research Center, Seattle, Washington. Sickle cell disease patient CD34⁺ HSPCs were isolated from plerixafor mobilized (IRB P00023325, FDA IND 131740) and β-thalassemia patient CD34⁺ HSPCs were isolated from unmobilized peripheral blood following Boston Children's Hospital institutional review board approval and patient informed consent. CD34⁺ HSPCs were enriched using the Miltenyi CD34 Microbead kit (Miltenyi Biotec). CD34⁺ HSPCs were thawed and cultured into X-VIVO 15 (Lonza, 04-418Q) supplemented with 100 ng m1⁻¹ human SCF, 100 ng ml⁻¹ human thrombopoietin (TPO) and 100 ng m1⁻¹ recombinant human Flt3-ligand (Flt3-L). HSPCs were electroporated with A3A (N57Q)-BE3 RNP 24 h after thawing. For in vitro erythroid differentiation experiments, 24 h after electroporation, HSPCs were transferred into erythroid differentiation medium (EDM) consisting of IMDM supplemented with 330 μg ml-holo-human transferrin, 10 μg ml-recombinant human insulin, 2 IU ml-heparin, 5% human solvent detergent pooled plasma AB, 3 IU ml⁻ erythropoietin, 1% L-glutamine, and 1% penicillin/streptomycin. During days 0-7 of culture, EDM was further supplemented with 10⁻⁶M hydrocortisone (Sigma), 100 ng m1⁻¹ human SCF, and 5 ng m1⁻¹ human IL-3 (R&D) as EDM-1. During days 7-11 of culture, EDM was supplemented with 100 ng m1⁻¹ human SCF only as EDM-2. During days 11-18 of culture, EDM had no additional supplements as EDM-3. HbF induction was assessed on day 18 of erythroid culture.

RNP Electroporation

Electroporation was performed using Lonza 4D Nucleofector (V4XP-3032 for 20 Nucleocuvette Strips or V4XP-3024 for 100 μl Nucleocuvettes) as the manufacturer's instructions. The modified synthetic sgRNA (2′-O-methyl 3′ phosphorothioate modifications in the first and last 3 nucleotides) was from Synthego. sgRNA concentration is calculated using the full-length product reporting method, which is 3-fold lower than the OD reporting method. CD34⁺ HSPCs were thawed 24 h before electroporation. For 20 μl Nucleocuvette Strips, the RNP complex was prepared by mixing A3A (N57Q)-BE3 protein (800 pmol) and sgRNA (800 pmol, full-length product reporting method) and incubating for 15 min at room temperature immediately before electroporation. 50 K HSPCs resuspended in 20 μl P3 solution were mixed with RNP and transferred to a cuvette for electroporation with program EO-100. For 100 μl cuvette electroporation, the RNP complex was made by mixing 4000 pmol A3A (N57Q)-BE3 protein and 4000 pmol sgRNA. 5M HSPCs were resuspended in 100 μl P3 solution for RNP electroporation as described above. For one cycle of electroporation, the electroporated cells were resuspended with X-VIVO medium with cytokines and changed into EDM 24 h later for in vitro differentiation. For two cycles of electroporation, the electroporated cells were maintained in X-VIVO medium with cytokines and do another electroporation 24 h later, the cells were cultured in X-VIVO medium with cytokines for 24 h then performed transplant or transferred to EDM. For mouse transplantation experiments, cells were maintained in X-VIVO 15 with cytokines for 24 h prior to infusion.

Measurement of Base Editing

Editing frequencies were measured with cells cultured in EDM 5 days after electroporation. Briefly, genomic DNA was extracted using the Qiagen Blood and Tissue kit. BCL11A enhancer DHS +58 core and HBB promoter −28 region were amplified with KOD Hot Start DNA Polymerase and corresponding primers using the following cycling conditions: 95 degrees for 3 min; 35 cycles of 95 degrees for 20 s, 60 degrees for 10 s, and 70 degrees for 10 s; 70 degrees for 5 min. Resulting PCR products were subjected to Sanger sequencing or Illumina deep sequencing. For Sanger sequencing, traces were imported to EditR software for base editing measurement. For deep sequencing, BCL11A enhancer loci or HBB promoter loci were amplified with corresponding primers firstly. After another round of PCR with primers containing sample-specific barcodes and adaptor, amplicons were sequenced for 2×150 paired-end reads with MiSeq Sequencing System (Illumina). Frequencies of editing outcomes were quantified using CRISPResso2 software³¹ (v2.0.30—quantification window center-10 —quantification window size 10 —base editor target C—base editor result T—base editor output TRUE) and collapsed based on mutations in the quantification window. The collapsed alleles were filtered such that only mutations overlapping the 17-18^{th} base pairs of the guide sequence were counted as indels, and the percent alleles passing filter was summed to obtain the total percent indels per sample. Base editing outcomes per sample were calculated by normalizing the post-alignment percent frequencies of each nucleotide to the total percent of aligned nucleotides at each locus of the spacer sequence.

RT-qPCR Quantification of Globin and BCL11A Expression

RNA isolation with RNeasy columns (Qiagen, 74106), reverse transcription with iScript cDNA synthesis kit (Bio-Rad, 170-8890), RT-qPCR with iQ SYBR Green Supermix (Bio-Rad, 170-8880) was subject to determine globin expression using primers amplifying HBG½ , HBB or HBA½ cDNA. BCL11A mRNA expression was determined by primers amplifying BCL11A or CAT as internal control. We used CAT as a reference transcript since it is both highly expressed and stable throughout erythroid maturation. All gene expression data represent the mean of at least three technical replicates.

Hemoglobin HPLC

Hemolysates were prepared from erythroid cells after 18 days of erythroid differentiation using Hemolysate reagent (5125, Helena Laboratories) and analyzed with D-10 Hemoglobin Analyzer (Bio-Rad). HbA2 and HbE cannot be distinguished by this method. Human erythroid cells were purified from xenotransplanted mouse bone marrow by CD235a microbead (130-050-501, Miltenyi Biotec) isolation prior to HPLC analysis.

Clonal Culture of CD34⁺ HSPCs

Edited CD34⁺ HSPCs were sorted into 180 μl EDM-1 in 96-well round bottom plates (Nunc) at one cell per well using FACSAria II. The cells were changed into EDM-2 media 7 days later in 24-well plates (Nunc). After additional 4 days of culture, the cells were changed into 300 μl-500 μl EDM-3 for further differentiation. After additional 7 days of culture, half of the cells were harvested for genotyping analysis and half for a single hemoglobin HPLC measurement per colony.

In Vitro Sickling and Microscopy Analysis

In vitro differentiated erythroid cells were stained with 2 μg ml⁻¹ of the cell-permeable DNA dye Hoechst 33342 (Life Technologies) and the enucleated cells which are negative for Hoechst 33342 were FACS sorted and subjected to in vitro sickling assay. Sickling was induced by adding 500 μl freshly prepared 1.5% sodium metabisulfite (MBS) solution prepared in PBS into enucleated cells resuspended with 500 μl EDM-3 in 24-well plate, followed by incubation at room temperature. Live cell images were acquired using a Nikon Eclipse Ti inverted microscope. Image acquisition was performed at room temperature and air in 24-well plate. Data were analyzed by paired two-tailed Student's t-test.

Human CD34⁺ HSPC Transplant and Flow Cytometry Analysis

All animal experiments were approved by the Boston Children's Hospital Institutional Animal Care and Use Committee. CD34⁺ HSPCs were obtained from deidentified healthy donors under protocols approved by the institutional review board of Boston Children's Hospital, with the informed consent of all participants, and complying with relevant ethical regulations. NOD.Cg-Kit^{W-41J}tyr⁺Prkdc^{scid}Il2g^{tm1Wjl} (NBSGW) mice were obtained from Jackson Laboratory (Stock 026622). Non-irradiated NBSGW female mice (4-5 weeks of age) were infused by retro-orbital injection with 0.8M CD34⁺ HSPCs (resuspended in 200 μl DPBS) derived from healthy donors. Bone marrow was isolated for human xenograft analysis 16 weeks post engraftment. Secondary transplants were conducted using retro-orbital injection of bone marrow cells from the primary recipients. For flow cytometry analysis of bone marrow, BM cells were first incubated with Human TruStain FcX (422302, BioLegend) and TruStain fcX ((anti-mouse CD16/32, 101320, BioLegend) blocking antibodies for 10 min, followed by the incubation with V450 Mouse Anti-Human CD45 Clone HI30 (560367, BD Biosciences), PE-eFluor 610 mCD45 Monoclonal Antibody (30-F11) (61-0451-82, Thermo Fisher), FITC anti-human CD235a Antibody (349104, BioLegend), PE anti-human CD33 Antibody (366608, BioLegend), APC anti-human CD19 Antibody (302212, BioLegend), FITC anti-human CD34 Antibody (343504, BioLegend), PE/Cy7 anti-human CD3 Antibody (300420, BioLegend) and Fixable Viability Dye eFluor 780 for live/dead staining (65-0865-14, Thermo Fisher). Percentage human engraftment was calculated as hCD45⁺ cells/(hCD45⁺ cells+mCD45⁺cells)×100. B cells (CD19⁺) was gated on the hCD45⁺ population. Granulocytes and monocytes were gated on the hCD45⁺hCD19⁻. Human erythroid cells (CD235a⁺) were gated on mCD45⁻hCD45⁻ population. Human HSPCs (CD34+Lin-) were gated on hCD45+hCD19-hCD33-population. For the staining with immunophenotype markers of HSCs, CD34⁺ HSPCs were incubated with Pacific Blue anti-human CD34 Antibody (343512, Biolegend), PE/Cy5 anti-human CD38 (303508, Biolegend), APC anti-human CD90 (328114, Biolegend), APC-H7 Mouse Anti-Human CD45RA (560674, BD Bioscience). Cell cycle phase in live CD34⁺ HSPCs was detected by flow cytometry as described previously⁸. Cells were resuspended in pre-warmed HSPC medium. First, we added Hoechst 33342 to a final concentration of 10 μg/ml and incubated at 37 degrees for 15 min. Then we added Pyronin Y directly to cells at a final concentration of 3 μg/ml and incubated at 37 degrees for 15 min. After washing with PBS, we performed flow cytometric analysis or cell sorting. Cell sorting was performed on a FACSAria II machine (BD Biosciences). Fraction of B cells, granulocytes, monocytes and HSPCs calculated as percentage of hCD45+ cells. Fraction of erythroid cells calculated as percentage of hCD45− mCD45− cells.

Flow Cytometry for Enucleation and Cell Size Analysis

For the enucleation analysis, cells were stained with 2 μg m1⁻¹ of the cell-permeable DNA dye Hoechst 33342 (Life Technologies) for 10 minutes at 37 degrees. The Hoechst 33342 negative cells were further gated for cell size analysis with Forward Scatter (FSC) A parameter. Median value of forward scatter intensity normalized by data from healthy donors collected at the same time was used to characterize the cell size.

Imaging Flow Cytometry Analysis

In vitro differentiated erythroid cells stained with Hoechst 33342 were resuspended with 150 μl DPBS for analysis with Imagestream X Mark II (Merck Millipore). Well-focused Hoechst negative single cells were gated for circularity analysis with IDEAS software. Cells with circularity score above 15 were further gated to exclude cell debris and aggregates. No fewer than 2000 gated cells were analyzed to obtain a median circularity score.

Protein Expression and Purification

For protein expression, RIPL-BL21 (DE3) competent cells transformed with A3A (N57Q)-BE3 plasmid were grown in TB media at 37° C. and switched to 15° C. Cells were induced by 1 mM isopropyl β-d-1-thiogalactopyranoside (IPTG) for 16-20 hours. Cell paste was collected and lysed in PBS buffer containing 500 mM NaCl and 10% glycerol using microfluidizer. The lysate was centrifuged at 18,000 g for 40 mins. Proteins were purified using nickel affinity, cation exchange, and Superdex 200 size exclusion columns sequentially. The purified protein was concentrated in 30 mM HEPES buffer of pH 7.4 containing 150 mM NaCl and 10% glycerol. Protein samples and fractions were separated using SDS-PAGE and stained using GelCode Blue Stain Reagent (Fisher).

### REFERENCES

1. Holt, N. et al. Human hematopoietic stem/progenitor cells modified by zinc-finger nucleases targeted to CCRS control HIV-1 in vivo. Nat. Biotechnol. 28, 839-847 (2010).

2. Genovese, P. et al. Targeted genome editing in human repopulating haematopoietic stem cells. Nature 510, 235-40 (2014).

3. Hoban, M. D. et al. Correction of the sickle-cell disease mutation in human hematopoietic stem/progenitor cells. Blood 125, 2597-604 (2015).

4. Dever, D. P. et al. CRISPR/Cas9β-globin gene targeting in human haematopoietic stem cells. Nature 539, 384-389 (2016).

5. Dewitt, M. A. et al. Selection-free genome editing of the sickle mutation in human adult hematopoietic stem/progenitor cells. Sci. Transl. Med. 8, 1-9 (2016).

6. Gundry, M. C. et al. Highly Efficient Genome Editing of Murine and Human Hematopoietic Progenitor Cells by CRISPR/Cas9. Cell Rep 17, 1453-1461 (2016).

7. Ravin, S. S. De et al. CRISPR-Cas9 gene repair of hematopoietic stem cells from patients with X-linked chronic granulomatous disease. Sci. Transl. Med. 9, 1-10 (2017).

8. Wu, Y. et al. Highly efficient therapeutic gene editing of human hematopoietic stem cells. Nature Medicine 25, 776-783 (2019).

9. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).

10. Nishida, K. et al. Targeted nucleotide editing using hybrid prokaryotic and vertebrate adaptive immune systems. Science 353, (2016).

11. Gaudelli, N. M. et al. Programmable base editing of A·T to G·C in genomic DNA without DNA cleavage. Nat. Publ. Gr. 551, 464-471 (2017).

12. Seo, H. & Kim, J. S. Towards therapeutic base editing. Nat. Med. 24, 1493-1495 (2018).

13. Gehrke, J. M. et al. An apobec3a-cas9 base editor with minimized bystander and off-target activities. Nature Biotechnology 36, 977 (2018).

14. Bauer, D. E. et al. An Erythroid Enhancer of BCL11A Subject to Genetic Variation Determines Fetal Hemoglobin Level. Science 342, 253-257 (2013).

15. Canver, M. C. et al. BCL11A enhancer dissection by Cas9-mediated in situ saturating mutagenesis. Nature 527, 192-197 (2015).

16. Eng, B. et al. Three new β-globin gene promoter mutations identified through newborn screening. Hemoglobin 31, 129-134 (2007).

17. Li, Z. et al. A novel promoter mutation (HBB: c.-75G>T) was identified as a cause of β+-thalassemia. Hemoglobin 39, 115-120 (2015).

18. Ponczs, M., Ballantine, M., Solowiejczyk, D., Bar, I. & Schwartz, E. Beta-Thalassemia in a Kurdish Jew. J. Biol. Chem. 257, 5994-5996 (1982).

19. Mohrin, M. et al. Hematopoietic stem cell quiescence promotes error-prone DNA repair and mutagenesis. Cell Stem Cell 7,174-185 (2010).

20. McIntosh, B. E. et al. Nonirradiated NOD,B6.SCID Il2rgamma-/- kitW41/W41 (NBSGW) mice support multilineage engraftment of human hematopoietic cells. Stem Cell Reports 4, 171-180 (2015).

21. Fiorini, C. et al. Developmentally-faithful and effective human erythropoiesis in immunodeficient and Kit mutant mice. 1-7 (2017). doi:10.1002/ajh.24805

22. Tebas, P. et al. Gene Editing of CCRS in Autologous CD4 T Cells of Persons Infected with HIV. N. Engl. J. Med. 370, 901-910 (2014).

23. Qasim, W. et al. Molecular remission of infant B-ALL after infusion of universal TALEN gene-edited CART cells. Sci Transl Med 9, (2017).

24. Rees, H. A. & Liu, D. R. Base editing: precision chemistry on the genome and transcriptome of living cells. Nat. Rev. Genet. 19, 770-788 (2018).

25. Kim, S., Kim, D., Cho, S. W., Kim, J. & Kim, J. S. Highly efficient RNA-guided genome editing in human cells via delivery of purified Cas9 ribonucleoproteins. Genome Res. 24, 1012-1019 (2014).

26. Tsai, S. Q. & Joung, J. K. Defining and improving the genome-wide specificities of CRISPR-Cas9 nucleases. Nat. Rev. Genet. 17,300-312 (2016).

27. Grunewald, J. et al. Transcriptome-wide off-target RNA editing induced by CRISPR-guided DNA base editors. Nature 569, 433-437 (2019).

28. Zuo, E. et al. Cytosine base editor generates substantial off-target single-nucleotide variants in mouse embryos. Science 126, eaav9973 (2019).

29. Grunewald, J., Zhou, R., Iyer, S., Lareau, C. A. & Garcia, S. P. CRISPR adenine and cytosine base editors with reduced RNA off-target activities. bioRxiv 1-25 (2019).

30. Kluesner, M. G. et al. EditR: A Method to Quantify Base Editing from Sanger Sequencing. Cris. J. 1, 239-250 (2018).

31. Clement, K. et al. CRISPResso2 provides accurate and rapid genome editing sequence analysis. Nat. Biotechnol. 37,224-226 (2019).

### Example 3

Potent HbF induction by BCL11A enhancer and HBG½ promoter multiplex base editing in human CD34+ HSPCs

One advantage of base editing is the opportunity to perform multiplex editing, that is to simultaneously generate DNA sequence changes at multiple genomic loci. The inventors specifically contemplated that targeting multiple sequences that serve to enforce HbF repression at both the BCL11A erythroid enhancer and HBG½ promoter by multiplex base editing would lead to especially robust HbF elevation. Positions at the core of the BCL11A erythroid enhancers +55 and +58 representing critical binding sites for the erythroid transcription factors GATA1 and TAL1, that could be base edited by an adenine base editor we identified. Further, positions at the HBG½ promoters that could be base edited by an adenine base editor and that overlap the known binding sites for the HbF repressors BCL11A and ZBTB7A and sites that are carry mutations in individuals with hereditary persistence of fetal hemoglobin (HPFH) were identified. Single guide RNA (sgRNA) spacer sequences shown below, also indicating if these match the top or bottom genomic strand.

SgRNA_BCL11A enh 58 spacer:
(SEQ ID NO: 42)
TTTATCACAGGCTCCAGGAA (bottom)
SgRNA_BCL11A enh 55 spacer:
(SEQ ID NO: 231)
CACTGATAGGGGTCGCGGTA (bottom)
SgRNA_HBG1/2_115 spacer:
(SEQ ID NO: 232)
CTTGACCAATAGCCTTGACA (bottom)
SgRNA_HBG1/2_198 spacer:
(SEQ ID NO: 233)
GTGGGGAAGGGGCCCCCAAG (top)

To achieve the most efficient HbF induction in human CD34+ HSPCs, these 4 sgRNAs were delivered to target simultaneously the BCL11A enhancer (at both the +58 and +55 enhancers) and HBG½ promoter sites (−115 and −198) simultaneously along with ABEmax (Koblan et al. 2018) mRNA via Lonza 4D electroporation (e.g., using 3.6 μM each of sgRNA and 25 μg of ABEmax mRNA in 100 μl of Lonza 4D system).

Twenty-four hours after electroporation, single cells were sorted using FACSAria II and subjected to 3 phase erythroid differentiation liquid culture over 18 days. On day 18, half of the cells were harvested for next generation sequencing genotyping analysis and half for a single hemoglobin HPLC measurement per clone. For bulk cells, cells were harvested 5 days after electroporation to measure editing by Sanger sequencing (Sanger sequencing results were analyzed by EditR software). Bulk cells were harvested 18 days after erythroid differentiation to detect HbF level by HPLC.

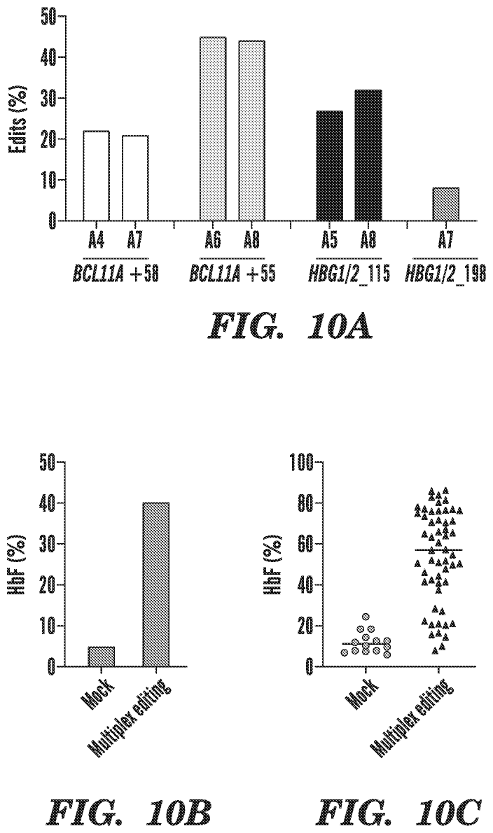

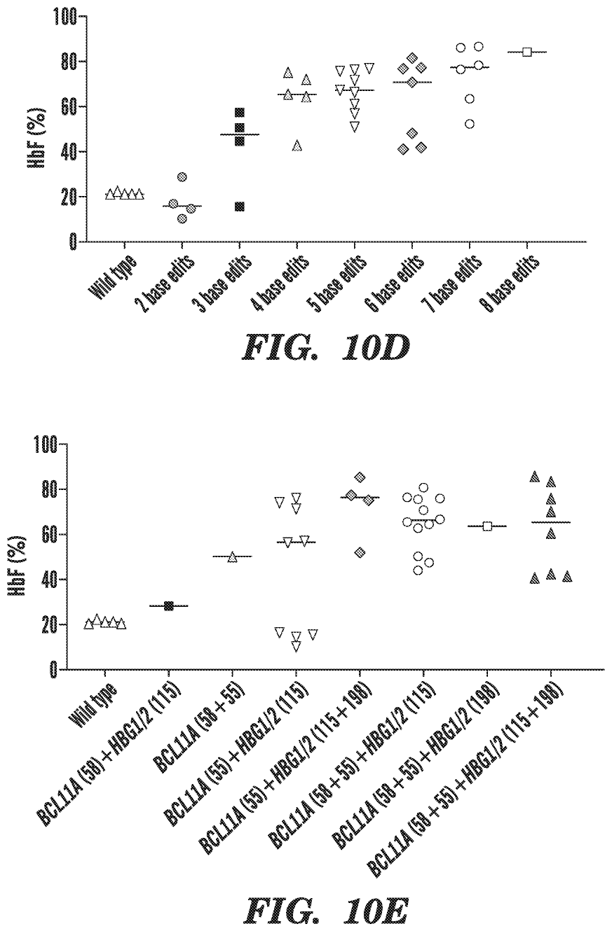

22% A>G editing at A4 and 21% A>G editing at A7 at +58 BCL11A enhancer, 45% editing at A6 and 44% editing at A8 at +58 BCL11A enhancer, 27% editing at A5 and 32% editing at A8 at HBG½ promoter −115, 8% editing at A7 at HBG½ promoter −198 was observed in bulk erythroid cells (FIG. 10a). Multiplex base editing resulted in potent HbF induction, 40.5% HbF compared with 4.9% of HbF level of mock in bulk erythroid cells (FIG. 10b). The HbF level in fifty-five multiplex edited clones showed a median of 57.2%, ranging from 8.1% to 86.7%, as compared to a median of 11.4% ranging from 6.2% to 24.7% in control unedited clones (FIG. 10c). To identify the correlation of HbF level and multiplex base edits, the HbF level of individual clones and their associated base edits at the four edited sites were further analyzed. There are 2 potential alleles to be edited at +58 and +55 BCL11A enhancer and 4 potential alleles to be edited at HBG½ promoter −115 and −198 sites following multiplex base editing (2 each at HBG1 and HBG2 promoters) described in Example 3. A dose-response relationship was observed between multiplex base edits and HbF induction. As compared to unedited clones with 21.2% HbF, three base edits resulted in a median of 47.6% HbF, 4 base edits gave a median of 65.2% HbF, 5 base edits had a median of 67.4% HbF, 6 base edits had a median of 70.8% HbF, 7 base edits had a median of 77.5% HbF, 8 base edits led to 84% HbF (FIG. 10d). Base edits targeting on 2 sites, +58 BCL11A enhancer and HBG½ promoter −115, +58 and +55 BCL11A enhancer or +55 BCL11A enhancer and HBG½ promoter −115, had a wide range of HbF levels (10.2%-76.9) depending on monoallelic or biallelic edits with them. Biallelic base editing was required for robust HbF induction at the BCL11A enhancer. Base edits targeting three sites, +55 BCL11A enhancer and HBG½ promoter −115 and −198, +58 and +55 BCL11A enhancer and HBG½ promoter −115, +58 and +55 BCL11A enhancer and HBG½ promoter −198, all had robust HbF induction ranging from 44.6%-86.3%, similar to base edits targeting on all four sites (FIG. 10e).

Overall, these results indicated that multiplex base editing targeting simultaneously BCL11A erythroid enhancer and HBG½ promoter sequences resulted in especially robust HbF induction.

### REFERENCES

Koblan L W, Doman J L, Wilson C, Levy J M, Tay T, Newby G A, Maianti J P, Raguram A, Liu D R. Improving cytidine and adenine base editors by expression optimization and ancestral reconstruction. Nat Biotechnol. 2018 Oct;36(9):843-846. doi: 10.1038/nbt.4172. Epub 2018 May 29.PMID: 29813047
Liu N, Hargreaves V V, Zhu Q, Kurland JV, Hong J, Kim W, Sher F, Macias-Trevino C, Rogers J M, Kurita R, Nakamura Y, Yuan G C, Bauer D E, Xu J, Bulyk M L, Orkin S H. Direct Promoter Repression by BCL11A Controls the Fetal to Adult Hemoglobin Switch. Cell. 2018 Apr 5;173(2):430-442.e17. doi: 10.1016/j.cell.2018.03.016. Epub 2018 Mar 29. PMID: 29606353
Martyn G E, Wienert B, Yang L, Shah M, Norton U, Burdach J, Kurita R, Nakamura Y, Pearson R C M, Funnell A P W, Quinlan K G R, Crossley M. Natural regulatory mutations elevate the fetal globin gene via disruption of BCL11A or ZBTB7A binding. Nat Genet. 2018 April; 50(4):498-503. doi: 10.1038/s41588-018-0085-0. Epub 2018 Apr 2. PMID: 29610478

Table 1 show sgRNA sequences that target the BCL11A enhancer DHS +62, +58, and +55 functional regions, the BCL11A exon 2, and/or a HBG½ promoter site. These sgRNA sequences produced HbF enrichment.

| | | | | | | Chr2 |
|---|---|---|---|---|---|---|
| SEQ | | | | | Coordinate | Genomic |
| ID | | | | Targeted | Relative | Coordinate |
| NO: | Identifer | sgRNA Sequence | PAM | Site | to TSS | (hgl9) |
| 1 | BCL_00108_H_D55 | TCTGAGGAGCTAGAGACTTG | NGG | DHS_55 | 54701 | 60725932 |
| 2 | BCL_00096_H_D55 | AGCAAATAGGCTTAGTGTGC | NGG | DHS_55 | 54874 | 60725759 |
| 3 | BCL_01427 HD55 | GGCTAAATAATGAATGTCCC | NGG | DHS_55 | 54944 | 60725689 |
| | | | RC | | | |
| 4 | BCL_00093_H_D55 | TCCCTTCCTAGAATTGGCCT | NGG | DHS_55 | 54950 | 60725683 |
| 5 | BCL_00092_H_D55 | TTCCCTTCCTAGAATTGGCC | NGG | DHS_55 | 54951 | 60725682 |
| 6 | BCL_01428_H_D55 | GAATGTCCCAGGCCAATTCT | NGG | DHS_55 | 54955 | 60725678 |
| | | | RC | | | |
| 7 | BCL_00091_H_D55 | CCCACTTCCCTTCCTAGAAT | NGG | DHS_55 | 54956 | 60725677 |
| 8 | BCL_00090_H_D55 | CCTGGTACCAGGAAGGCAAT | NGG | DHS_55 | 54989 | 60725644 |
| 9 | BCL_00089_H_D55 | TCCTGGTACCAGGAAGGCAA | NGG | DHS_55 | 54990 | 60725643 |
| 10 | BCL_00088_H_D55 | GCATCATCCTGGTACCAGGA | NGG | DHS_55 | 54996 | 60725637 |
| 11 | BCL_00087_H_D55 | CATTGCATCATCCTGGTACC | NGG | DHS_55 | 55000 | 60725633 |
| 12 | BCL_00086_H_D55 | CTCCAAGCATTGCATCATCC | NGG | DHS_55 | 55007 | 60725626 |
| 13 | BCL_01438_H_D55 | TACCAGGATGATGCAATGCT | NGG | DHS_55 | 55016 | 60725617 |
| | | | RC | | | |
| 14 | BCL_00085_H_D55 | GGGTGTGCCCTGAGAAGGTG | NGG | DHS_55 | 55040 | 60725593 |
| 15 | BCL_00084_H_D55 | AGGGTGTGCCCTGAGAAGGT | NGG | DHS_55 | 55041 | 60725592 |
| 16 | BCL_00082_H_D55 | TCACAGGGTGTGCCCTGAGA | NGG | DHS_55 | 55045 | 60725588 |
| 17 | BCL_01443_H_D55 | GGCACACCCTGTGATCTTGT | NGG | DHS_55 | 55065 | 60725568 |
| | | | RC | | | |
| 18 | BCL_00073_H_D55 | AGCACACAAGATGCACACCC | NGG | DHS_55 | 55096 | 60725537 |
| 19 | BCL_01448_H_D55 | TGTGCTTGGTCGGCACTGAT | NGG | DHS_55 | 55124 | 60725509 |
| | | | RC | | | |
| 20 | BCL_01449_H_D55 | GTGCTTGGTCGGCACTGATA | NGG | DHS_55 | 55125 | 60725508 |
| | | | RC | | | |
| 21 | BCL_01450_H_D55 | TGCTTGGTCGGCACTGATAG | NGG | DHS_55 | 55126 | 60725507 |
| | | | RC | | | |
| 22 | BCL_01454_H_D55 | GGGTCGCGGTAGGGAGTTGT | NGG | DHS_55 | 55146 | 60725487 |
| | | | RC | | | |
| 23 | BCL_00065_H_D55 | GCCAACAGTGATAACCAGCA | NGG | DHS_55 | 55235 | 60725398 |
| 24 | BCL_00064_H_D55 | TGCCAACAGTGATAACCAGC | NGG | DHS_55 | 55236 | 60725397 |
| 25 | BCL_01461_H_D55 | GCCCTGCTGGTTATCACTGT | NGG | DHS_55 | 55245 | 60725388 |
| | | | RC | | | |
| 26 | BCL_00062_H_D55 | AGCAGCCCTGGGCACAGAAG | NGG | DHS_55 | 55272 | 60725361 |
| 27 | BCL_00058_H_D55 | CCTCTATGTAGACGGGTGTG | NGG | DHS_55 | 55311 | 60725322 |
| 28 | BCL_00057_H_D55 | GGAAGGGCCTCTATGTAGAC | NGG | DHS_55 | 55318 | 60725315 |
| 29 | BCL_00051_H_D55 | GGAGGTGTGGAGGGGATAAC | NGG | DHS_55 | 55356 | 60725277 |
| 30 | BCL_00031_H_D55 | CTGGCAGACCCTCAAGAGCA | NGG | DHS_55 | 55444 | 60725189 |
| 31 | BCL_00027_H_D55 | CCCATGGAGGTGGGGAGATG | NGG | DHS_55 | 55474 | 60725159 |
| 32 | BCL_01483_H_D55 | GTCATCCTCGGCCAATGAAG | NGG | DHS_55 | 55559 | 60725074 |
| | | | RC | | | |
| 33 | BCL_00012_H_D55 | AAGTGAGCCAGGTGATAGAA | NGG | DHS_55 | 55585 | 60725048 |
| 34 | BCL 00008 HD55 | TGAAACCAAGCTTCCTCTGC | NGG | DHS_55 | 55612 | 60725021 |
| 35 | BCL_01495_H_D55 | AGGGAGAAATGAGACAAAAG | NGG | DHS_55 | 55700 | 60724933 |
| | | | RC | | | |
| 36 | BCL_01497_H_D55 | AAGAGGCCACTGAGTCCTTT | NGG | DHS_55 | 55717 | 60724916 |
| | | | RC | | | |
| 37 | BCL_01615_H_D58 | ACTCTTAGACATAACACACC | NGG | DHS_58 | 58176 | 60722457 |
| | | | RC | | | |
| 38 | BCL_01616_H_D58 | CTCTTAGACATAACACACCA | NGG | DHS_58 | 58177 | 60722456 |
| | | | RC | | | |
| 39 | BCL_01617_H_D58 | CTAACAGTTGCTTTTATCAC | NGG | DHS_58 | 58232 | 60722401 |
| | | | RC | | | |
| 40 | BCL_01618_H_D58 | TTGCTTTTATCACAGGCTCC | NGG | DHS_58 | 58239 | 60722394 |
| | | | RC | | | |
| 41 | BCL_01619_H_D58 | TTTTATCACAGGCTCCAGGA | NGG | DHS_58 | 58243 | 60722390 |
| | | | RC | | | |
| 42 | BCL_01620_H_D58 | TTTATCACAGGCTCCAGGAA | NGG | DHS_58 | 58244 | 60722389 |
| | | | RC | | | |
| 43 | BCL_00187_H_D58 | ATCAGAGGCCAAACCCTTCC | NGG | DHS_58 | 58246 | 60722387 |
| 44 | BCL_00188_H_D58 | CTTCAAAGTTGTATTGACCC | NGG | DHS_58 | 58183 | 60722450 |
| 45 | BCL_01621_H_D58 | CACAGGCTCCAGGAAGGGTT | NGG | DHS_58 | 58249 | 60722384 |
| | | | RC | | | |
| 46 | BCL_00186_H_D58 | CACGCCCCCACCCTAATCAG | NGG | DHS_58 | 58261 | 60722372 |
| 47 | BCL_01622_H_D58 | GAAGGGTTTGGCCTCTGATT | NGG | DHS_58 | 58261 | 60722372 |
| | | | RC | | | |
| 48 | BCL_01623_H_D58 | AAGGGTTTGGCCTCTGATTA | NGG | DHS_58 | 58262 | 60722371 |
| | | | RC | | | |
| 49 | BCL_01624_H_D58 | GGTTTGGCCTCTGATTAGGG | NGG | DHS_58 | 58265 | 60722368 |
| | | | RC | | | |
| 50 | BCL_01625_H_D58 | GTTTGGCCTCTGATTAGGGT | NGG | DHS_58 | 58266 | 60722367 |
| | | | RC | | | |
| 51 | BCL_01626_H_D58 | TTTGGCCTCTGATTAGGGTG | NGG | DHS_58 | 58267 | 60722366 |
| | | | RC | | | |
| 52 | BCL_01627_H_D58 | TTGGCCTCTGATTAGGGTGG | NGG | DHS_58 | 58268 | 60722365 |
| | | | RC | | | |
| 53 | BCL_01629_H_D58 | TCTGATTAGGGTGGGGGCGT | NGG | DHS_58 | 58274 | 60722359 |
| | | | RC | | | |
| 54 | BCL_01631_H_D58 | ATTAGGGTGGGGGCGTGGGT | NGG | DHS_58 | 58278 | 60722355 |
| | | | RC | | | |
| 55 | BCL_01632_H_D58 | TTAGGGTGGGGGCGTGGGTG | NGG | DHS_58 | 58279 | 60722354 |
| | | | RC | | | |
| 56 | BCL_01634_H_D58 | TGGGTGGGGTAGAAGAGGAC | NGG | DHS_58 | 58293 | 60722340 |
| | | | RC | | | |
| 57 | BCL_00185_H_D58 | GCAAACGGCCACCGATGGAG | NGG | DHS_58 | 58309 | 60722324 |
| 58 | BCL_00184_H_D58 | CCTGGGCAAACGGCCACCGA | NGG | DHS_58 | 58314 | 60722319 |
| 59 | BCL_00183_H_D58 | AAGAGGCCCCCCTGGGCAAA | NGG | DHS_58 | 58324 | 60722309 |
| 60 | BCL_01637_H_D58 | CCATCGGTGGCCGTTTGCCC | NGG | DHS_58 | 58325 | 60722308 |
| | | | RC | | | |
| 61 | BCL_01638_H_D58 | CATCGGTGGCCGTTTGCCCA | NGG | DHS_58 | 58326 | 60722307 |
| | | | RC | | | |
| 62 | BCL_01639_H_D58 | ATCGGTGGCCGTTTGCCCAG | NGG | DHS_58 | 58327 | 60722306 |
| | | | RC | | | |
| 63 | BCL_01640_H_D58 | TCGGTGGCCGTTTGCCCAGG | NGG | DHS_58 | 58328 | 60722305 |
| | | | RC | | | |
| 64 | BCL_01641_H_D58 | CGGTGGCCGTTTGCCCAGGG | NGG | DHS_58 | 58329 | 60722304 |
| | | | RC | | | |
| 65 | BCL_00182_H_D58 | CTTCCGAAAGAGGCCCCCCT | NGG | DHS_58 | 58331 | 60722302 |
| 66 | BCL_00181_H_D58 | CCTTCCGAAAGAGGCCCCCC | NGG | DHS_58 | 58332 | 60722301 |
| 67 | BCL_00160_H_D58 | TCAGGGGGAGGCAAGTCAGT | NGG | DHS_58 | 58575 | 60722058 |
| 68 | BCL_00154_H_D58 | AGGGAAAAGGGAGAGGAAAA | NGG | DHS_58 | 58612 | 60722021 |
| 69 | BCL_01665_H_D58 | TGTAACTAATAAATACCAGG | NGG | DHS_58 | 58706 | 60721927 |
| | | | RC | | | |
| 70 | BCL_01669_H_D58 | CCAGCTGAAGAAAGAACATT | NGG | DHS_58 | 58870 | 60721763 |
| | | | RC | | | |
| 71 | BCL_00135_H_D58 | CCATCTCCCTAATCTCCAAT | NGG | DHS_58 | 58958 | 60721675 |
| 72 | BCL_00131_H_D58 | TGGGGAGAGAAGAGTGGAAA | NGG | DHS_58 | 59030 | 60721603 |
| 73 | BCL_00130_H_D58 | GGAGTATGGGGAGAGAAGAG | NGG | DHS_58 | 59036 | 60721597 |
| 74 | BCL_01684_H_D58 | ACAACCTCCTTGTTTACAGA | NGG | DHS_58 | 59129 | 60721504 |
| | | | RC | | | |
| 75 | BCL_01788_H_D62 | GAGATTTACTCTTGTTGCCC | NGG | DHS_62 | 61848 | 60718785 |
| | | | RC | | | |
| 76 | BCL_01790_H_D62 | TTGCCCGGGCTGGAATGCAA | NGG | DHS_62 | 61862 | 60718771 |
| | | | RC | | | |
| 77 | BCL_00245_H_D62 | GGAGATCGCTTGAACCTGGG | NGG | DHS_62 | 61901 | 60718732 |
| 78 | BCL_00241_H_D62 | CTCAGCTACTCGGGAGGCTG | NGG | DHS_62 | 61926 | 60718707 |
| 79 | BCL_00240_H_D62 | TGTAATCTCAGCTACTCGGG | NGG | DHS_62 | 61932 | 60718701 |
| 80 | BCL_00239_H_D62 | GCCTGTAATCTCAGCTACTC | NGG | DHS_62 | 61935 | 60718698 |
| 81 | BCL_00238_H_D62 | TGCCTGTAATCTCAGCTACT | NGG | DHS_62 | 61936 | 60718697 |
| 82 | BCL_01794_H_D62 | CAGGCATGTATTACCATGCC | NGG | DHS_62 | 61964 | 60718669 |
| | | | RC | | | |
| 83 | BCL_00233_H_D62 | CAGGAGGATCACCTGAGGTC | NGG | DHS_62 | 62037 | 60718596 |
| 84 | BCL_01799_H_D62 | CTCAGGTGATCCTCCTGCCC | NGG | DHS_62 | 62054 | 60718579 |
| | | | RC | | | |
| 85 | BCL_00229_H_D62 | CCCAGCACTTTGGGAGGCCG | NGG | DHS_62 | 62060 | 60718573 |
| 86 | BCL_00228_H_D62 | TCCCAGCACTTTGGGAGGCC | NGG | DHS_62 | 62061 | 60718572 |
| 87 | BCL_00227_H_D62 | ATCCCAGCACTTTGGGAGGC | NGG | DHS_62 | 62062 | 60718571 |
| 88 | BCL_00225_H_D62 | ACCTGTAATCCCAGCACTTT | NGG | DHS_62 | 62069 | 60718564 |
| 89 | BCL_01800_H_D62 | GCCCCGGCCTCCCAAAGTGC | NGG | DHS_62 | 62070 | 60718563 |
| | | | RC | | | |
| 90 | BCL_01801_H_D62 | CCCCGGCCTCCCAAAGTGCT | NGG | DHS_62 | 62071 | 60718562 |
| | | | RC | | | |
| 91 | BCL_01825_H_D62 | ATTTGCTCTTCTCCAGGGTG | NGG | DHS_62 | 62469 | 60718164 |
| | | | RC | | | |
| 92 | BCL_00210_H_D62 | TAAACAGCCACCCCACACCC | NGG | DHS_62 | 62470 | 60718163 |
| 93 | BCL_01826_H_D62 | TTTGCTCTTCTCCAGGGTGT | NGG | DHS_62 | 62470 | 60718163 |
| | | | RC | | | |
| 94 | BCL_01828_H_D62 | CTCTTCTCCAGGGTGTGGGG | NGG | DHS_62 | 62474 | 60718159 |
| | | | RC | | | |
| 95 | BCL_01829_H_D62 | TGTGGGGTGGCTGTTTAAAG | NGG | DHS_62 | 62487 | 60718146 |
| | | | RC | | | |
| 96 | BCL_01831_H_D62 | GGGTGGCTGTTTAAAGAGGG | NGG | DHS_62 | 62491 | 60718142 |
| | | | RC | | | |
| 97 | BCL_01833_H_D62 | AGTTCAAGTAGATATCAGAA | NGG | DHS_62 | 62580 | 60718053 |
| | | | RC | | | |
| 98 | BCL_01834_H_D62 | TATCAGAAGGGAACTGTTTG | NGG | DHS_62 | 62592 | 60718041 |
| | | | RC | | | |
| 99 | BCL_02015_H_exon2 | AAGAATGGCTTCAAGAGGCT | NGG | exon2 | 7218 | 60773415 |
| | | | RC | | | |
| 100 | BCL_02014_H_exon2 | TCTGTAAGAATGGCTTCAAG | NGG | exon2 | 7223 | 60773410 |
| | | | RC | | | |
| 101 | BCL_00248_H_exon2 | ACAGATGATGAACCAGACCA | NGG | exon2 | 7224 | 60773409 |
| 102 | BCL_00249_H_exon2 | TGAACCAGACCACGGCCCGT | NGG | exon2 | 7232 | 60773401 |
| 103 | BCL_00250_H_exon2 | GAACCAGACCACGGCCCGTT | NGG | exon2 | 7233 | 60773400 |
| 104 | BCL_00251_H_exon2 | GGCCCGTTGGGAGCTCCAGA | NGG | exon2 | 7245 | 60773388 |
| 105 | BCL_00252_H_exon2 | GCCCGTTGGGAGCTCCAGAA | NGG | exon2 | 7246 | 60773387 |
| 106 | BCL_00253_H_exon2 | CCCGTTGGGAGCTCCAGAAG | NGG | exon2 | 7247 | 60773386 |
| 107 | BCL_02011_H_exon2 | CTGGAGCTCCCAACGGGCCG | NGG | exon2 | 7258 | 60773375 |
| | | | RC | | | |
| 108 | BCL_02010_H_exon2 | CCCCTTCTGGAGCTCCCAAC | NGG | exon2 | 7264 | 60773369 |
| | | | RC | | | |
| 109 | BCL_02009_H_exon2 | TCCCCTTCTGGAGCTCCCAA | NGG | exon2 | 7265 | 60773368 |
| | | | RC | | | |
| 110 | BCL_00254_H_exon2 | GATCATGACCTCCTCACCTG | NGG | exon2 | 7269 | 60773364 |
| 111 | BCL_00255_H_exon2 | ATCATGACCTCCTCACCTGT | NGG | exon2 | 7270 | 60773363 |
| 112 | BCL_02008_H_exon2 | AGGAGGTCATGATCCCCTTC | NGG | exon2 | 7277 | 60773356 |
| | | | RC | | | |
| 113 | BCL_02007_H_exon2 | GGCACTGCCCACAGGTGAGG | NGG | exon2 | 7294 | 60773339 |
| | | | RC | | | |
| 114 | BCL_00256_H_exon2 | GTGCCAGATGAACTTCCCAT | NGG | exon2 | 7295 | 60773338 |
| 115 | BCL_00257_H_exon2 | TGCCAGATGAACTTCCCATT | NGG | exon2 | 7296 | 60773337 |
| 116 | BCL_00258_H_exon2 | GCCAGATGAACTTCCCATTG | NGG | exon2 | 7297 | 60773336 |
| 117 | BCL_02006_H_exon2 | TCTGGCACTGCCCACAGGTG | NGG | exon2 | 7297 | 60773336 |
| | | | RC | | | |
| 118 | BCL_00259_H_exon2 | CCAGATGAACTTCCCATTGG | NGG | exon2 | 7298 | 60773335 |
| 119 | BCL_02005_H_exon2 | GTTCATCTGGCACTGCCCAC | NGG | exon2 | 7302 | 60773331 |
| | | | RC | | | |
| 120 | BCL_02004_H_exon2 | CCCCCAATGGGAAGTTCATC | NGG | exon2 | 7315 | 60773318 |
| | | | RC | | | |
| 121 | BCL_02003_H_exon2 | AAATAAGAATGTCCCCCAAT | NGG | exon2 | 7327 | 60773306 |
| | | | RC | | | |
| 122 | BCL_02002_H_exon2 | AAAATAAGAATGTCCCCCAA | NGG | exon2 | 7328 | 60773305 |
| | | | RC | | | |
| 123 | BCL_00261_H_exon2 | CACAAACGGAAACAATGCAA | NGG | exon2 | 7341 | 60773292 |
| 124 | BCL_00262_H_exon2 | CCTCTGCTTAGAAAAAGCTG | NGG | exon2 | 7367 | 60773266 |
| 125 | BCL_02001_H_exon2 | CCACAGCTTTTTCTAAGCAG | NGG | exon2 | 7384 | 60773249 |
| | | | RC | | | |
| 126 | BCL_02000_H_exon2 | TCGATTGGTGAAGGGGAAGG | NGG | exon2 | 7412 | 60773221 |
| | | | RC | | | |
| 127 | BCL_01999_H_exon2 | ATCTCGATTGGTGAAGGGGA | NGG | exon2 | 7415 | 60773218 |
| | | | RC | | | |
| 128 | BCL_01998_H_exon2 | TTTCATCTCGATTGGTGAAG | NGG | exon2 | 7419 | 60773214 |
| | | | RC | | | |
| 129 | BCL_00263_H_exon2 | GAAAAAAGCATCCAATCCCG | NGG | exon2 | 7421 | 60773212 |
| 130 | BCL_00264_H_exon2 | AAAAGCATCCAATCCCGTGG | NGG | exon2 | 7424 | 60773209 |
| 131 | BCL_00265_H_exon2 | GCATCCAATCCCGTGGAGGT | NGG | exon2 | 7428 | 60773205 |
| 132 | BCL_00266_H_exon2 | TCCCGTGGAGGTTGGCATCC | NGG | exon2 | 7436 | 60773197 |
| 133 | BCL_00267_H_exon2 | TGGCATCCAGGTCACGCCAG | NGG | exon2 | 7448 | 60773185 |
| 134 | BCL_01994_H_exon2 | GATGCCAACCTCCACGGGAT | NGG | exon2 | 7449 | 60773184 |
| | | | RC | | | |
| 135 | BCL_01993_H_exon2 | ACCTGGATGCCAACCTCCAC | NGG | exon2 | 7454 | 60773179 |
| | | | RC | | | |
| 136 | BCL_01992_H_exon2 | GACCTGGATGCCAACCTCCA | NGG | exon2 | 7455 | 60773178 |
| | | | RC | | | |
| 137 | BCL_01991_H_exon2 | CGTCATCCTCTGGCGTGACC | NGG | exon2 | 7471 | 60773162 |
| | | | RC | | | |
| 138 | BCL_01990_H_exon2 | GATAAACAATCGTCATCCTC | NGG | exon2 | 7481 | 60773152 |
| | | | RC | | | |
| 139 | BCL_01989_H_exon2 | CTGCTATGTGTTCCTGTTTG | NGG | exon2 | 7525 | 60773108 |
| | | | RC | | | |
| 231 | SgRNA_BCL11A enh | CACTGATAGGGGTCGCGGTA | | DHS_55 | | |
| | 55 spacer | | | | | |
| 232 | SgRNA_HBG1/2_115 | CTTGACCAATAGCCTTGACA | | HBG1/2 | | |
| | spacer | | | promoter | | |
| | | | | site -115 | | |
| 233 | SgRNA_HBG1/2_198 | GTGGGGAAGGGGCCCCCAAG | | HBG1/2 | | |
| | spacer | | | promoter | | |
| | | | | site -198 | | |

Table 2 show subset of sgRNA sequences from Table 1 that target DHS 58+ functional region

| Seq ID No | sgRNA | 20-nt protospacer sequence |
|---|---|---|
| 37 | 1615 | ACTCTTAGACATAACACACC |
| 38 | 1616 | CTCTTAGACATAACACACCA |
| 39 | 1617 | CTAACAGTTGCTTTTATCAC |
| 40 | 1618 | TTGCTTTTATCACAGGCTCC |
| 41 | 1619 | TTTTATCACAGGCTCCAGGA |
| 42 | 1620 | TTTATCACAGGCTCCAGGAA |
| 43 | 0187 | ATCAGAGGCCAAACCCTTCC |
| 44 | 0188 | CTTCAAAGTTGTATTGACCC |
| 45 | 1621 | CACAGGCTCCAGGAAGGGTT |
| 46 | 0186 | CACGCCCCCACCCTAATCAG |
| 47 | 1622 | GAAGGGTTTGGCCTCTGATT |
| 48 | 1623 | AAGGGTTTGGCCTCTGATTA |
| 50 | 1625 | GTTTGGCCTCTGATTAGGGT |
| 51 | 1626 | TTTGGCCTCTGATTAGGGTG |
| 52 | 1627 | TTGGCCTCTGATTAGGGTGG |
| 53 | 1629 | TCTGATTAGGGTGGGGGCGT |
| 54 | 1631 | ATTAGGGTGGGGGCGTGGGT |
| 55 | 1632 | TTAGGGTGGGGGCGTGGGTG |
| 56 | 1634 | TGGGTGGGGTAGAAGAGGAC |
| 57 | 0185 | GCAAACGGCCACCGATGGAG |
| 62 | 1639 | ATCGGTGGCCGTTTGCCCAG |

Table 3 show HBB genotype of β-thalassemia patients.

| Simplified | |
|---|---|
| genotype | Genotype |
| β⁰β⁰ | Homozygous codon 44 (-C) TCC > TC- frameshift β⁰ |
| | thalassemia mutation |
| β⁺β⁰_{#1} | IVSII-745 C > G; codon 44 (-C) TCC > TC- |
| β⁺β⁰_{#2} | IVS I-110 G > A; codon 39 CAG > TAG or Gln39Term |
| β⁺β⁺ | Homozygous IVS-I-5 G > C |
| (^{A}γδβ)⁰β⁰ | Large Chinese (^{A}γδβ)⁰ deletion; codon 41/42 (--CTTT) |
| β^{E}β⁰_{#1} | Codon 26 (G > A; GAG > AAG; Glu > Lys) hemoglobin E; |
| | codon 71/72 + A frameshift β⁰ thalassemia mutation |
| β^{E}β⁰_{#2} | Codon 26 (G > A; GAG > AAG; Glu > Lys) hemoglobin E; |
| | p. Ser72fsX2 |

Table 4 shows primers used in the Example section.

Primers fA3:B19s for in vitro transcription of sgRNAs. Protospacer
sequence are bolded and underlined.

| | |
|---|---|
| IVT-1615-F | TAATACGACTCACTATAGGGACTCTTAGACATAACACACCGTTTTAGAGCTAGAA |
| | (SEQ ID NO: 140) |
| IVT-1616-F | TAATACGACTCACTATAGGGCTCTTAGACATAACACACCAGTTTTAGAGCTAGAA |
| | (SEQ ID NO: 141) |
| IVT-1617-F | TAATACGACTCACTATAGGGTACAGAGCCCCAGTCCTGGAGTTTTAGAGCTAGAA |
| | (SEQ ID NO: 142) |
| IVT-1618-F | TAATACGACTCACTATAGGGTTGCTTTTATCACAGGCTCCGTTTTAGAGCTAGAA |
| | (SEQ ID NO: 143) |
| IVT-1619-F | TAATACGACTCACTATAGGGTTTTATCACAGGCTCCAGGAGTTTTAGAGCTAGAA |
| | (SEQ ID NO: 144) |
| IVT-1620-F | TAATACGACTCACTATAGGGTTTATCACAGGCTCCAGGAAGTTTTAGAGCTAGAA |
| | (SEQ ID NO: 145) |
| IVT-0186-F | TAATACGACTCACTATAGGGCACGCCCCCACCCTAATCAGGTTTTAGAGCTAGAA |
| | (SEQ ID NO: 146) |
| IVT-0187-F | TAATACGACTCACTATAGGGATCAGAGGCCAAACCCTTCCGTTTTAGAGCTAGAA |
| | (SEQ ID NO: 147) |
| IVT-1621-F | TAATACGACTCACTATAGGGCACAGGCTCCAGGAAGGGTTGTTTTAGAGCTAGAA |
| | (SEQ ID NO: 148) |
| IVT-1622-F | TAATACGACTCACTATAGGGGAAGGGTTTGGCCTCTGATTGTTTTAGAGCTAGAA |
| | (SEQ ID NO: 149) |
| IVT-1623-F | TAATACGACTCACTATAGGGAAGGGTTTGGCCTCTGATTAGTTTTAGAGCTAGAA |
| | (SEQ ID NO: 150) |
| IVT-1625-F | TAATACGACTCACTATAGGGGTTTGGCCTCTGATTAGGGTGTTTTAGAGCTAGAA |
| | (SEQ ID NO: 151) |
| IVT-1626-F | TAATACGACTCACTATAGGGTTTGGCCTCTGATTAGGGTGGTTTTAGAGCTAGAA |
| | (SEQ ID NO: 152) |
| IVT-1627-F | TAATACGACTCACTATAGGGTTGGCCTCTGATTAGGGTGGGTTTTAGAGCTAGAA |
| | (SEQ ID NO: 153) |
| IVT-1629-F | TAATACGACTCACTATAGGGTCTGATTAGGGTGGGGGCGTGTTTTAGAGCTAGAA |
| | (SEQ ID NO: 154) |
| IVT-1631-F | TAATACGACTCACTATAGGGATTAGGGTGGGGGCGTGGGTGTTTTAGAGCTAGAA |
| | (SEQ ID NO: 155) |
| IVT-1632-F | TAATACGACTCACTATAGGGTTAGGGTGGGGGCGTGGGTGGTTTTAGAGCTAGAA |
| | (SEQ ID NO: 156) |
| IVT-1634-F | TAATACGACTCACTATAGGGTGGGTGGGGTAGAAGAGGACGTTTTAGAGCTAGAA |
| | (SEQ ID NO: 157) |
| IVT-0185-F | TAATACGACTCACTATAGGGGCAAACGGCCACCGATGGAGGTTTTAGAGCTAGAA |
| | (SEQ ID NO: 158) |
| IVT-1639-F | TAATACGACTCACTATAGGGATCGGTGGCCGTTTGCCCAGGTTTTAGAGCTAGAA |
| | (SEQ ID NO: 159) |
| IVT-AAVS1-F | TAATACGACTCACTATAGGGCTCCCTCCCAGGATCCTCTCGTTTTAGAGCTAGAA |
| | (SEQ ID NO: 160) |
| IVT-sg-R | AAAAAAGCACCGACTCGGTG (SEQ ID NO: 161) |

Primers for TIDE analysis.

| | |
|---|---|
| 58 enh-1F | GAGAGTGCAGACAGGGGAAG (SEQ ID NO: 161) |
| 58 enh-1R | ACCCTGGAAAACAGCCTGAC (SEQ ID NO: 162) |
| 58 enh-2F | CACACGGCATGGCATACAAA (SEQ ID NO: 163) |
| 58 enh-2R | CACCCTGGAAAACAGCCTGA (SEQ ID NO: 164) |
| AAVS1-1F | CACCTTATATTCCCAGGGCCG (SEQ ID NO: 165) |
| AAVS1-1R | CCTAGGACGCACCATTCTCAC (SEQ ID NO: 166) |
| AAVS1-2F | ATTGGGTCTAACCCCCACCT (SEQ ID NO: 167) |
| AAVS1-2R | TCAGTGAAACGCACCAGACA (SEQ ID NO: 168) |

Primers for RT-qPCR.

| | |
|---|---|
| BCL11A_RT_e1/e2_114F | AACCCCAGCACTTAAGCAAA (SEQ ID NO: 169) |
| BCL11A_RT_e1/e2_114R | GGAGGTCATGATCCCCTTCT (SEQ ID NO: 170) |
| HBA_RT_F | GCCCTGGAGAGGATGTTC (SEQ ID NO: 171) |
| HBA_RT_R | TTCTTGCCGTGGCCCTTA (SEQ ID NO: 172) |
| HBB_RT_F | CAGTGCAGGCTGCCTATC (SEQ ID NO: 173) |
| HBB_RT_R | ATACTTGTGGGCCAGGGCAT (SEQ ID NO: 174) |
| HBG_RT_F | TGGGTCATTTCACAGAGGAG (SEQ ID NO: 175) |
| HBG_RT_R | CATCTTCCACATTCACCTTGC (SEQ ID NO: 176) |
| GAPDH_RT_125_F | ACCCAGAAGACTGTGGATGG (SEQ ID NO: 177) |
| GAPDH_RT_125_R | TTCAGCTCAGGGATGACCTT (SEQ ID NO: 178) |
| hCAT_RT90_e8_F | CTTCGACCCAAGCAACATGC (SEQ ID NO: 179) |
| hCAT_RT90_e8_R | CGGTGAGTGTCAGGATAGGC (SEQ ID NO: 180) |

Primers for deep sequencing.

| | |
|---|---|
| 58 enh_seq_1F | GCCAGAAAAGAGATATGGCATC (SEQ ID NO: 181) |
| 58 enh_seq_1R | AGAGAGCCTTCCGAAAGAGG (SEQ ID NO: 182) |
| OT1_seq_F | GTTCTCTCTTCTTCCTGACAGTG (SEQ ID NO: 183) |
| OT1_seq_R | GAGGTCCCTATGAAAAGATGGCT (SEQ ID NO: 184) |
| OT2_seq_F | GTTGAATGCCAAGTGCCCAA (SEQ ID NO: 185) |
| OT2_seq_R | GGTCTCAGTTCAGCCCCTTC (SEQ ID NO: 186) |
| OT3_seq_F | TGAACAATATTGCCTTTTGTGCT (SEQ ID NO: 187) |
| OT3_seq_R | ATGCTGCTGTAAGGCACTGT (SEQ ID NO: 188) |
| OT4_seq_F | TCCATAAAACAATGTTGAGGTGGG (SEQ ID NO: 189) |
| OT4_seq_R | GTCCTCCAGTTGATCCTGAAGT (SEQ ID NO: 190) |
| OT5_seq_F | ATCCAGGGGCTTTGAGATTGA (SEQ ID NO: 191) |
| OT5_seq_R | TCTCCATCCCCGTTGTTAAGTGA (SEQ ID NO: 192) |
| OT6_seq_F | CACACACAAAGCCCTTCTGC (SEQ ID NO: 193) |
| OT6_seq_R | CACCATATCCAGCCTGTCGG (SEQ ID NO: 194) |
| OT7_seq_F | CTCTGGAAAGCAGGGACCAT (SEQ ID NO: 195) |
| OT7_seq_R | GCATGCTAACCAGCACACTG (SEQ ID NO: 196) |
| OT8_seq_F | ACAGAGCTGGGCCAATAACC (SEQ ID NO: 197) |
| OT8_seq_R | TGTTCACATGGGAAAGCCTCA (SEQ ID NO: 198) |
| OT9_seq_F | GCTTGTGTGCGTGTATGGAA (SEQ ID NO: 199) |
| OT9_seq_R | GTAGCACACTAATACATGGAAATGA (SEQ ID NO: 200) |
| OT10_seq_F | TAAGATCCAAACACCCAATCACG (SEQ ID NO: 201) |
| OT10_seq_R | AGTCTTCAGCTGGTGATTTCAGG (SEQ ID NO: 202) |
| OT11_seq_F | GTACCATTCCTCCAACTAAGGCA (SEQ ID NO: 203) |
| OTll_seq_R | CTAAGTTCTTCCACCTCGAGTCAT (SEQ ID NO: 204) |
| OT12_seq_F | AGCTGAGATCACACCACTGC (SEQ ID NO: 205) |
| OT12_seq_R | TCTAGCCCCTGGTAACCTCC (SEQ ID NO: 206) |
| OT13_seq_F | ACAGAAGATGAATAGCGGAACA (SEQ ID NO: 207) |
| OT13_seq_R | TAGTGACGCGAATAGCCCTG (SEQ ID NO: 208) |
| OT14_seq_F | ATCAGCCTGGATCCCTACCC (SEQ ID NO: 209) |
| OT14_seq_R | ACCTGAGATTCCTCTGGGCT (SEQ ID NO: 210) |
| OT15_seq_F | TCTGTTTCATGGTGATGCTTGA (SEQ ID NO: 211) |
| OT15_seq_R | ACAATTTTTCACTCTTGGTACTCTT (SEQ ID NO: 212) |
| OT16_seq_F | TCTCTCCTTAGATTTCTTCATGTCT (SEQ ID NO: 213) |
| OT16_seq_R | CAGAACAAAGGCACAGCCAC (SEQ ID NO: 214) |
| OT17_seq_F | ACAGAATGCTATGAGAGACGTT (SEQ ID NO: 215) |
| OT17_seq_R | GGTAGGAAAAACGCAGAAAAGA (SEQ ID NO: 216) |
| OT18_seq_F | CATGACTTGGTGAAGCCCCT (SEQ ID NO: 217) |
| OT18_seq_R | ATTTGGGCCACCAACTTAGG (SEQ ID NO: 218) |
| OT19_seq_F | GTGTAGTGGAGGAGACAGACAA (SEQ ID NO: 219) |
| OT19_seq_R | TTTCAAACTTGCCAGACCCCA (SEQ ID NO: 220) |
| OT20_seq_F | GTTCCCAGCATCCCAAAAGAAC (SEQ ID NO: 221) |
| OT20_seq_R | GGAGGCTCTGAGAGAATGAGG (SEQ ID NO: 222) |
| OT21_seq_F | GATGTGTTCAATGCAATGGAGATTA (SEQ ID NO: 223) |
| OT21_seq_R | GGCTGCCTCTCATTCTCTTGTA (SEQ ID NO: 224) |
| OT22_seq_F | AGAGTCCAATAAATTGAGGTTTCAC (SEQ ID NO: 225) |
| OT22_seq_R | ATAACTCCATTCCGGGAGCC (SEQ ID NO: 226) |
| OT23_seq_F | CAGGGCCTCACTTTTGCCTC (SEQ ID NO: 227) |
| OT23_seq_R | TGACTGCATATCCATGCACCAT (SEQ ID NO: 228) |
| OT24_seq_F | CAGCTGAGGCTACTGCTGTT (SEQ ID NO: 229) |
| OT24_seq_R | TCTTCTGTTCACTCTTTGGCT (SEQ ID NO: 230) |

SEQ ID NO: 234 provides the nucleotide sequence chromosome 2 location 60725424 to 60725688 (+55 functional region).

(SEQ ID NO: 234)
GACACTGAAGGCTGGGCACAGCCTTGGGGACCGCTCACAGGACATGCAGC
AGTGTGTGCCGACAACTCCCTACCGCGACCCCTATCAGTGCCGACCAAGC
ACACAAGATGCACACCCAGGCTGGGCTGGACAGAGGGGTCCCACAAGATC
ACAGGGTGTGCCCTGAGAAGGTGGGGAGCTCACAGCCTCCAAGCATTGCA
TCATCCTGGTACCAGGAAGGCAATGGGCTGCCCCATACCCACTTCCCTTC
CTAGAATTGGCCTGG

SEQ ID NO: 235 provides the nucleotide sequence for chromosome 2 location 60722238 to 60722466 (+58 functional region).

(SEQ ID NO: 235)
TTCATTCCCATTGAGAAATAAAATCCAATTCTCCATCACCAAGAGAGCCT
TCCGAAAGAGGCCCCCCTGGGCAAACGGCCACCGATGGAGAGGTCTGCCA
GTCCTCTTCTACCCCACCCACGCCCCCACCCTAATCAGAGGCCAAACCCT
TCCTGGAGCCTGTGATAAAAGCAACTGTTAGCTTGCACTAGACTAGCTTC
AAAGTTGTATTGACCCTGGTGTGTTATGT

SEQ ID NO: 236 provides the nucleotide sequence for chromosome 2 location 60718042 to 60718186 (+62 functional region).

(SEQ ID NO: 236)
ATTTCCCTTCTGATATCTACTTGAACTTTCAGATAAAAAAAAAAAAGCAA
GTTGCAGTAACATGTTATGCTACACAAAGATTAGCATGAATATCCACCCT
CTTTAAACAGCCACCCCACACCCTGGAGAAGAGCAAATGTGAAGT

SEQ ID NO: 237 provides the nucleotide sequence for BCL11A exon2 (dna range -chr2:60773106-60773435)

(SEQ ID NO: 237)
CTGCTATGTGTTCCTGTTTGGGGCAAATTCCTCTAGATGACGTTGATAAA
CAATCGTCATCCTCTGGCGTGACCTGGATGCCAACCTCCACGGGATTGGA
TGCTTTTTTCATCTCGATTGGTGAAGGGGAAGGTGGCTTATCCACAGCTT
TTTCTAAGCAGAGGCTGCCATTGCATTGTTTCCGTTTGTGCTCGATAAAA
ATAAGAATGTCCCCCAATGGGAAGTTCATCTGGCACTGCCCACAGGTGAG
GAGGTCATGATCCCCTTCTGGAGCTCCCAACGGGCCGTGGTCTGGTTCAT
CATCTGTAAGAATGGCTTCAAGAGGCTCGG

SEQ ID NO: 238 provides the nucleotide sequence for HBG1 promoter (dna range=chr11:5271088-5271387)

(SEQ ID NO: 238)
GTGGAACTGCTGAAGGGTGCTTCCTTTTATTCTTCATCCCTAGCCAGCCG
CCGGCCCCTGGCCTCACTGGATACTCTAAGACTATTGGTCAAGTTTGCCT
TGTCAAGGCTATTGGTCAAGGCAAGGCTGGCCAACCCATGGGTGGAGTTT
AGCCAGGGACCGTTTCAGACAGATATTTGCATTGAGATAGTGTGGGGAAG
GGGCCCCCAAGAGGATACTGCTAATTTTTTTTATAGCCTTTGCCTTGTTC
CGATTCAGTCATTCCAGTTTTTCTCTAATTTATTCTTCCCTTTAGCTAGT

SEQ ID NO: 239 provides the nucleotide sequence for HBG2 promoter (dna range=chr11:5276012-527631)

(SEQ ID NO: 239)
GTGGAACTGCTGAAGGGTGCTTCCTTTTATTCTTCATCCCTAGCCAGCCG
CCGGCCCCTGGCCTCACTGGATACTCTAAGACTATTGGTCAAGTTTGCCT
TGTCAAGGCTATTGGTCAAGGCAAGGCTGGCCAACCCATGGGTGGAGTTT
AGCCAGGGACCGTTTCAGACAGATATTTGCATTGAGATAGTGTGGGGAAG
GGGCCCCCAAGAGGATACTGCTGCTTAATTTTTTTTATAGCCTTTGCCTT
GTTCCGATTCAGTCATTCCAATTTTTCTCTAATTTATTCTTCCCTTTAGC

1. A ribonucleoprotein (RNP) complex comprising:
a. a base editor protein; and
b. a nucleic acid sequence shown in Table 1, SEQ ID NOS: 1-139, wherein there is at least one chemical modification to a nucleotide in the nucleic acid sequence.

2. The RNP complex of claim 1, wherein the nucleic acid sequence excludes the entire BCL11A enhancer functional regions and excludes the entire BCL11A coding region,
includes the entire BCL11A enhancer functional regions and excludes the entire BCL11A coding region, or
excludes the entire region between the human chromosome 2 location 60725424 to 60725688 (DHS +55 functional region), or excludes the entire region at location 60722238 to 60722466 (DHS +58 functional region), or excludes the entire region at location 60718042 to 60718186 (DHS +62 functional region), or excludes the entire region at location 60773106 to 60773435 (exon 2) of the human chromosome 2 is that according to UCSC Genome Browser hg 19 human genome assembly.

3. (canceled)

4. The RNP complex of claim 1, wherein the nucleic acid sequence is selected from the group consisting of SEQ ID NOS: 37, 38, 39, 40, 41, 42, 43, 44, 45, 46, 47, 48, 50, 51, 52, 53, 54, 55, 56, 57, and 62 as shown in Table 2.

5. The RNP complex of claim 1, wherein the chemical modification
a) is located at one or more terminal nucleotides in nucleic acid sequence,
b) is located only at the 3′ end, or added only at the 5′ end, or added at both the 5′ and 3′ ends of the nucleic acid sequence, or
c) is located to first three nucleotides and to the last three nucleotides of the nucleic acid sequence.

6. The RNP complex of claim 5, wherein the chemical modification is selected from the group consisting of 2′-β-methyl 3′phosphorothioate (MS), 2′-β-methyl-3′-phosphonoacetate (MP), 2′-β-Ci-4alkyl, 2′-H, 2′-β-Ci.3alkyl-β-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, 0, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag).

7. (canceled)

8. (canceled)

9. The RNP complex of claim 1, wherein the nucleic acid sequence further comprising a crRNA/tracrRNA sequence.

10. The RNP complex of claim 1, wherein the nucleic acid sequence is a single guide RNA (sgRNA).

11. (canceled)

12. The RNP complex of claim 1, wherein the base editor protein is a third generation base editor.

13. The RNP complex of claim 1, wherein the base editor protein is A3A (N57Q)-BE3, A3A-BE3, or A3A (N57G)-BE3.

14-39. (canceled)

40. A ribonucleoprotein (RNP) complex comprising:
a. base editor A3A (N57Q)-BE3; and
b. a nucleic acid sequences having the sequence of SEQ IN: 42, wherein there is at least one chemical modification to a nucleotide in the nucleic acid sequence.

41. A composition comprising a RNP complex of claim 1.

42-47. (canceled)

48. A method for producing a progenitor cell or a population of progenitor cells having decreased BCL11A mRNA or protein expression, the method comprising contacting an isolated progenitor cell with an effective amount of an RNP complex of claim 1, whereby the contacted cells or the differentiated progeny cells therefrom have decreased BCL11A mRNA or protein expression.

49. The method of claim 48, wherein the contacted progenitor cell acquires at least one genetic modification in the human chromosome 2 is that according to UCSC Genome Browser hg 19 human genome assembly.

50. The method of claim 49, wherein the at least one genetic modification is
a) a deletion, insertion or substitution of the genetic sequence of the cell,
b) a C to T, G, or A substitution,
c) any base substitution, or
d) located between chromosome 2 location 60725424 to 60725688 (+55 functional region), or at location 60722238 to 60722466 (+58 functional region), or at location 60718042 to 60718186 (+62 functional region), or at location 60773106 to 60773435 (exon 2) of the human chromosome 2 is that according to UCSC Genome Browser hg 19 human genome assembly,
e) located in at least one target selected from the group consisting of BCL11A erythroid enhancer DHS +62 functional region, BCL11A erythroid enhancer DHS +58 functional region, BCL11A erythroid enhancer DHS +55 functional region, BCL11A exon 2 region, HBG½ promoter site −115, and HBG½ promoter site −198, and/or
f) results in the increase in fetal hemoglobin induction.

51-56. (canceled)

57. A method of increasing fetal hemoglobin levels in a cell, the method comprising the steps of: contacting an isolated cell with an effective amount of a composition of an RNP complex of claim 1, whereby fetal hemoglobin expression is increased in said cell, or its progeny, relative to said cell prior to said contacting.

58. The method of claim 57, wherein the isolated progenitor cell or isolated cell is a hematopoietic progenitor cell or a hematopoietic stem cell, or induced pluripotent stem cell.

59. The method of claim 58, wherein the hematopoietic progenitor is a cell of the erythroid lineage.

60. (canceled)

61. The method of claim 57, wherein the isolated cell is contacted ex vivo or in vitro.

62. The method of claim 57, wherein the contacted cell acquires at least one genetic modification.

63-68. (canceled)

69. An isolated genetic engineered human cell or a population of genetic engineered human cells contacting with the RNP complex of claim 1.

70-76. (canceled)


SEQUENCE LISTING (249 sequences)

SEQ ID NO: 1 - DNA, 20 bp, Artificial Sequence
  Description of Artificial Sequence Synthetic oligonucleotide
tctgaggagctagagacttg

SEQ ID NO: 2 - DNA, 20 bp, Artificial Sequence
  Description of Artificial Sequence Synthetic oligonucleotide
agcaaataggcttagtgtgc

SEQ ID NO: 3 - DNA, 20 bp, Artificial Sequence
  Description of Artificial Sequence Synthetic oligonucleotide
ggctaaataatgaatgtccc

SEQ ID NO: 4 - DNA, 20 bp, Artificial Sequence
  Description of Artificial Sequence Synthetic oligonucleotide
tcccttcctagaattggcct

SEQ ID NO: 5 - DNA, 20 bp, Artificial Sequence
  Description of Artificial Sequence Synthetic oligonucleotide
ttcccttcctagaattggcc

SEQ ID NO: 6 - DNA, 20 bp, Artificial Sequence
  Description of Artificial Sequence Synthetic oligonucleotide
gaatgtcccaggccaattct

SEQ ID NO: 7 - DNA, 20 bp, Artificial Sequence
  Description of Artificial Sequence Synthetic oligonucleotide
cccacttcccttcctagaat

SEQ ID NO: 8 - DNA, 20 bp, Artificial Sequence
  Description of Artificial Sequence Synthetic oligonucleotide
cctggtaccaggaaggcaat

SEQ ID NO: 9 - DNA, 20 bp, Artificial Sequence
  Description of Artificial Sequence Synthetic oligonucleotide
tcctggtaccaggaaggcaa

SEQ ID NO: 10 - DNA, 20 bp, Artificial Sequence
  Description of Artificial Sequence Synthetic oligonucleotide
gcatcatcctggtaccagga

SEQ ID NO: 11 - DNA, 20 bp, Artificial Sequence
  Description of Artificial Sequence Synthetic oligonucleotide
cattgcatcatcctggtacc

SEQ ID NO: 12 - DNA, 20 bp, Artificial Sequence
  Description of Artificial Sequence Synthetic oligonucleotide
ctccaagcattgcatcatcc

SEQ ID NO: 13 - DNA, 20 bp, Artificial Sequence
  Description of Artificial Sequence Synthetic oligonucleotide
taccaggatgatgcaatgct

SEQ ID NO: 14 - DNA, 20 bp, Artificial Sequence
  Description of Artificial Sequence Synthetic oligonucleotide
gggtgtgccctgagaaggtg

SEQ ID NO: 15 - DNA, 20 bp, Artificial Sequence
  Description of Artificial Sequence Synthetic oligonucleotide
agggtgtgccctgagaaggt

SEQ ID NO: 16 - DNA, 20 bp, Artificial Sequence
  Description of Artificial Sequence Synthetic oligonucleotide
tcacagggtgtgccctgaga

SEQ ID NO: 17 - DNA, 20 bp, Artificial Sequence
  Description of Artificial Sequence Synthetic oligonucleotide
ggcacaccctgtgatcttgt

SEQ ID NO: 18 - DNA, 20 bp, Artificial Sequence
  Description of Artificial Sequence Synthetic oligonucleotide
agcacacaagatgcacaccc

SEQ ID NO: 19 - DNA, 20 bp, Artificial Sequence
  Description of Artificial Sequence Synthetic oligonucleotide
tgtgcttggtcggcactgat

SEQ ID NO: 20 - DNA, 20 bp, Artificial Sequence
  Description of Artificial Sequence Synthetic oligonucleotide
gtgcttggtcggcactgata

SEQ ID NO: 21 - DNA, 20 bp, Artificial Sequence
  Description of Artificial Sequence Synthetic oligonucleotide
tgcttggtcggcactgatag

SEQ ID NO: 22 - DNA, 20 bp, Artificial Sequence
  Description of Artificial Sequence Synthetic oligonucleotide
gggtcgcggtagggagttgt

SEQ ID NO: 23 - DNA, 20 bp, Artificial Sequence
  Description of Artificial Sequence Synthetic oligonucleotide
gccaacagtgataaccagca

SEQ ID NO: 24 - DNA, 20 bp, Artificial Sequence
  Description of Artificial Sequence Synthetic oligonucleotide
tgccaacagtgataaccagc

SEQ ID NO: 25 - DNA, 20 bp, Artificial Sequence
  Description of Artificial Sequence Synthetic oligonucleotide
gccctgctggttatcactgt

SEQ ID NO: 26 - DNA, 20 bp, Artificial Sequence
  Description of Artificial Sequence Synthetic oligonucleotide
agcagccctgggcacagaag

SEQ ID NO: 27 - DNA, 20 bp, Artificial Sequence
  Description of Artificial Sequence Synthetic oligonucleotide
cctctatgtagacgggtgtg

SEQ ID NO: 28 - DNA, 20 bp, Artificial Sequence
  Description of Artificial Sequence Synthetic oligonucleotide
ggaagggcctctatgtagac

SEQ ID NO: 29 - DNA, 20 bp, Artificial Sequence
  Description of Artificial Sequence Synthetic oligonucleotide
ggaggtgtggaggggataac

SEQ ID NO: 30 - DNA, 20 bp, Artificial Sequence
  Description of Artificial Sequence Synthetic oligonucleotide
ctggcagaccctcaagagca

SEQ ID NO: 31 - DNA, 20 bp, Artificial Sequence
  Description of Artificial Sequence Synthetic oligonucleotide
cccatggaggtggggagatg

SEQ ID NO: 32 - DNA, 20 bp, Artificial Sequence
  Description of Artificial Sequence Synthetic oligonucleotide
gtcatcctcggccaatgaag

SEQ ID NO: 33 - DNA, 20 bp, Artificial Sequence
  Description of Artificial Sequence Synthetic oligonucleotide
aagtgagccaggtgatagaa

SEQ ID NO: 34 - DNA, 20 bp, Artificial Sequence
  Description of Artificial Sequence Synthetic oligonucleotide
tgaaaccaagcttcctctgc

SEQ ID NO: 35 - DNA, 20 bp, Artificial Sequence
  Description of Artificial Sequence Synthetic oligonucleotide
agggagaaatgagacaaaag

SEQ ID NO: 36 - DNA, 20 bp, Artificial Sequence
  Description of Artificial Sequence Synthetic oligonucleotide
aagaggccactgagtccttt

SEQ ID NO: 37 - DNA, 20 bp, Artificial Sequence
  Description of Artificial Sequence Synthetic oligonucleotide
actcttagacataacacacc

SEQ ID NO: 38 - DNA, 20 bp, Artificial Sequence
  Description of Artificial Sequence Synthetic oligonucleotide
ctcttagacataacacacca

SEQ ID NO: 39 - DNA, 20 bp, Artificial Sequence
  Description of Artificial Sequence Synthetic oligonucleotide
ctaacagttgcttttatcac

SEQ ID NO: 40 - DNA, 20 bp, Artificial Sequence
  Description of Artificial Sequence Synthetic oligonucleotide
ttgcttttatcacaggctcc

SEQ ID NO: 41 - DNA, 20 bp, Artificial Sequence
  Description of Artificial Sequence Synthetic oligonucleotide
ttttatcacaggctccagga

SEQ ID NO: 42 - DNA, 20 bp, Artificial Sequence
  Description of Artificial Sequence Synthetic oligonucleotide
tttatcacaggctccaggaa

SEQ ID NO: 43 - DNA, 20 bp, Artificial Sequence
  Description of Artificial Sequence Synthetic oligonucleotide
atcagaggccaaacccttcc

SEQ ID NO: 44 - DNA, 20 bp, Artificial Sequence
  Description of Artificial Sequence Synthetic oligonucleotide
cttcaaagttgtattgaccc

SEQ ID NO: 45 - DNA, 20 bp, Artificial Sequence
  Description of Artificial Sequence Synthetic oligonucleotide
cacaggctccaggaagggtt

SEQ ID NO: 46 - DNA, 20 bp, Artificial Sequence
  Description of Artificial Sequence Synthetic oligonucleotide
cacgcccccaccctaatcag

SEQ ID NO: 47 - DNA, 20 bp, Artificial Sequence
  Description of Artificial Sequence Synthetic oligonucleotide
gaagggtttggcctctgatt

SEQ ID NO: 48 - DNA, 20 bp, Artificial Sequence
  Description of Artificial Sequence Synthetic oligonucleotide
aagggtttggcctctgatta

SEQ ID NO: 49 - DNA, 20 bp, Artificial Sequence
  Description of Artificial Sequence Synthetic oligonucleotide
ggtttggcctctgattaggg

SEQ ID NO: 50 - DNA, 20 bp, Artificial Sequence
  Description of Artificial Sequence Synthetic oligonucleotide
gtttggcctctgattagggt

SEQ ID NO: 51 - DNA, 20 bp, Artificial Sequence
  Description of Artificial Sequence Synthetic oligonucleotide
tttggcctctgattagggtg

SEQ ID NO: 52 - DNA, 20 bp, Artificial Sequence
  Description of Artificial Sequence Synthetic oligonucleotide
ttggcctctgattagggtgg

SEQ ID NO: 53 - DNA, 20 bp, Artificial Sequence
  Description of Artificial Sequence Synthetic oligonucleotide
tctgattagggtgggggcgt

SEQ ID NO: 54 - DNA, 20 bp, Artificial Sequence
  Description of Artificial Sequence Synthetic oligonucleotide
attagggtgggggcgtgggt

SEQ ID NO: 55 - DNA, 20 bp, Artificial Sequence
  Description of Artificial Sequence Synthetic oligonucleotide
ttagggtgggggcgtgggtg

SEQ ID NO: 56 - DNA, 20 bp, Artificial Sequence
  Description of Artificial Sequence Synthetic oligonucleotide
tgggtggggtagaagaggac

SEQ ID NO: 57 - DNA, 20 bp, Artificial Sequence
  Description of Artificial Sequence Synthetic oligonucleotide
gcaaacggccaccgatggag

SEQ ID NO: 58 - DNA, 20 bp, Artificial Sequence
  Description of Artificial Sequence Synthetic oligonucleotide
cctgggcaaacggccaccga

SEQ ID NO: 59 - DNA, 20 bp, Artificial Sequence
  Description of Artificial Sequence Synthetic oligonucleotide
aagaggcccccctgggcaaa

SEQ ID NO: 60 - DNA, 20 bp, Artificial Sequence
  Description of Artificial Sequence Synthetic oligonucleotide
ccatcggtggccgtttgccc

SEQ ID NO: 61 - DNA, 20 bp, Artificial Sequence
  Description of Artificial Sequence Synthetic oligonucleotide
catcggtggccgtttgccca

SEQ ID NO: 62 - DNA, 20 bp, Artificial Sequence
  Description of Artificial Sequence Synthetic oligonucleotide
atcggtggccgtttgcccag

SEQ ID NO: 63 - DNA, 20 bp, Artificial Sequence
  Description of Artificial Sequence Synthetic oligonucleotide
tcggtggccgtttgcccagg

SEQ ID NO: 64 - DNA, 20 bp, Artificial Sequence
  Description of Artificial Sequence Synthetic oligonucleotide
cggtggccgtttgcccaggg

SEQ ID NO: 65 - DNA, 20 bp, Artificial Sequence
  Description of Artificial Sequence Synthetic oligonucleotide
cttccgaaagaggcccccct

SEQ ID NO: 66 - DNA, 20 bp, Artificial Sequence
  Description of Artificial Sequence Synthetic oligonucleotide
ccttccgaaagaggcccccc

SEQ ID NO: 67 - DNA, 20 bp, Artificial Sequence
  Description of Artificial Sequence Synthetic oligonucleotide
tcagggggaggcaagtcagt

SEQ ID NO: 68 - DNA, 20 bp, Artificial Sequence
  Description of Artificial Sequence Synthetic oligonucleotide
agggaaaagggagaggaaaa

SEQ ID NO: 69 - DNA, 20 bp, Artificial Sequence
  Description of Artificial Sequence Synthetic oligonucleotide
tgtaactaataaataccagg

SEQ ID NO: 70 - DNA, 20 bp, Artificial Sequence
  Description of Artificial Sequence Synthetic oligonucleotide
ccagctgaagaaagaacatt

SEQ ID NO: 71 - DNA, 20 bp, Artificial Sequence
  Description of Artificial Sequence Synthetic oligonucleotide
ccatctccctaatctccaat

SEQ ID NO: 72 - DNA, 20 bp, Artificial Sequence
  Description of Artificial Sequence Synthetic oligonucleotide
tggggagagaagagtggaaa

SEQ ID NO: 73 - DNA, 20 bp, Artificial Sequence
  Description of Artificial Sequence Synthetic oligonucleotide
ggagtatggggagagaagag

SEQ ID NO: 74 - DNA, 20 bp, Artificial Sequence
  Description of Artificial Sequence Synthetic oligonucleotide
acaacctccttgtttacaga

SEQ ID NO: 75 - DNA, 20 bp, Artificial Sequence
  Description of Artificial Sequence Synthetic oligonucleotide
gagatttactcttgttgccc

SEQ ID NO: 76 - DNA, 20 bp, Artificial Sequence
  Description of Artificial Sequence Synthetic oligonucleotide
ttgcccgggctggaatgcaa

SEQ ID NO: 77 - DNA, 20 bp, Artificial Sequence
  Description of Artificial Sequence Synthetic oligonucleotide
ggagatcgcttgaacctggg

SEQ ID NO: 78 - DNA, 20 bp, Artificial Sequence
  Description of Artificial Sequence Synthetic oligonucleotide
ctcagctactcgggaggctg

SEQ ID NO: 79 - DNA, 20 bp, Artificial Sequence
  Description of Artificial Sequence Synthetic oligonucleotide
tgtaatctcagctactcggg

SEQ ID NO: 80 - DNA, 20 bp, Artificial Sequence
  Description of Artificial Sequence Synthetic oligonucleotide
gcctgtaatctcagctactc

SEQ ID NO: 81 - DNA, 20 bp, Artificial Sequence
  Description of Artificial Sequence Synthetic oligonucleotide
tgcctgtaatctcagctact

SEQ ID NO: 82 - DNA, 20 bp, Artificial Sequence
  Description of Artificial Sequence Synthetic oligonucleotide
caggcatgtattaccatgcc

SEQ ID NO: 83 - DNA, 20 bp, Artificial Sequence
  Description of Artificial Sequence Synthetic oligonucleotide
caggaggatcacctgaggtc

SEQ ID NO: 84 - DNA, 20 bp, Artificial Sequence
  Description of Artificial Sequence Synthetic oligonucleotide
ctcaggtgatcctcctgccc

SEQ ID NO: 85 - DNA, 20 bp, Artificial Sequence
  Description of Artificial Sequence Synthetic oligonucleotide
cccagcactttgggaggccg

SEQ ID NO: 86 - DNA, 20 bp, Artificial Sequence
  Description of Artificial Sequence Synthetic oligonucleotide
tcccagcactttgggaggcc

SEQ ID NO: 87 - DNA, 20 bp, Artificial Sequence
  Description of Artificial Sequence Synthetic oligonucleotide
atcccagcactttgggaggc

SEQ ID NO: 88 - DNA, 20 bp, Artificial Sequence
  Description of Artificial Sequence Synthetic oligonucleotide
acctgtaatcccagcacttt

SEQ ID NO: 89 - DNA, 20 bp, Artificial Sequence
  Description of Artificial Sequence Synthetic oligonucleotide
gccccggcctcccaaagtgc

SEQ ID NO: 90 - DNA, 20 bp, Artificial Sequence
  Description of Artificial Sequence Synthetic oligonucleotide
ccccggcctcccaaagtgct

SEQ ID NO: 91 - DNA, 20 bp, Artificial Sequence
  Description of Artificial Sequence Synthetic oligonucleotide
atttgctcttctccagggtg

SEQ ID NO: 92 - DNA, 20 bp, Artificial Sequence
  Description of Artificial Sequence Synthetic oligonucleotide
taaacagccaccccacaccc

SEQ ID NO: 93 - DNA, 20 bp, Artificial Sequence
  Description of Artificial Sequence Synthetic oligonucleotide
tttgctcttctccagggtgt

SEQ ID NO: 94 - DNA, 20 bp, Artificial Sequence
  Description of Artificial Sequence Synthetic oligonucleotide
ctcttctccagggtgtgggg

SEQ ID NO: 95 - DNA, 20 bp, Artificial Sequence
  Description of Artificial Sequence Synthetic oligonucleotide
tgtggggtggctgtttaaag

SEQ ID NO: 96 - DNA, 20 bp, Artificial Sequence
  Description of Artificial Sequence Synthetic oligonucleotide
gggtggctgtttaaagaggg

SEQ ID NO: 97 - DNA, 20 bp, Artificial Sequence
  Description of Artificial Sequence Synthetic oligonucleotide
agttcaagtagatatcagaa

SEQ ID NO: 98 - DNA, 20 bp, Artificial Sequence
  Description of Artificial Sequence Synthetic oligonucleotide
tatcagaagggaactgtttg

SEQ ID NO: 99 - DNA, 20 bp, Artificial Sequence
  Description of Artificial Sequence Synthetic oligonucleotide
aagaatggcttcaagaggct

SEQ ID NO: 100 - DNA, 20 bp, Artificial Sequence
  Description of Artificial Sequence Synthetic oligonucleotide
tctgtaagaatggcttcaag

SEQ ID NO: 101 - DNA, 20 bp, Artificial Sequence
  Description of Artificial Sequence Synthetic oligonucleotide
acagatgatgaaccagacca

SEQ ID NO: 102 - DNA, 20 bp, Artificial Sequence
  Description of Artificial Sequence Synthetic oligonucleotide
tgaaccagaccacggcccgt

SEQ ID NO: 103 - DNA, 20 bp, Artificial Sequence
  Description of Artificial Sequence Synthetic oligonucleotide
gaaccagaccacggcccgtt

SEQ ID NO: 104 - DNA, 20 bp, Artificial Sequence
  Description of Artificial Sequence Synthetic oligonucleotide
ggcccgttgggagctccaga

SEQ ID NO: 105 - DNA, 20 bp, Artificial Sequence
  Description of Artificial Sequence Synthetic oligonucleotide
gcccgttgggagctccagaa

SEQ ID NO: 106 - DNA, 20 bp, Artificial Sequence
  Description of Artificial Sequence Synthetic oligonucleotide
cccgttgggagctccagaag

SEQ ID NO: 107 - DNA, 20 bp, Artificial Sequence
  Description of Artificial Sequence Synthetic oligonucleotide
ctggagctcccaacgggccg

SEQ ID NO: 108 - DNA, 20 bp, Artificial Sequence
  Description of Artificial Sequence Synthetic oligonucleotide
ccccttctggagctcccaac

SEQ ID NO: 109 - DNA, 20 bp, Artificial Sequence
  Description of Artificial Sequence Synthetic oligonucleotide
tccccttctggagctcccaa

SEQ ID NO: 110 - DNA, 20 bp, Artificial Sequence
  Description of Artificial Sequence Synthetic oligonucleotide
gatcatgacctcctcacctg

SEQ ID NO: 111 - DNA, 20 bp, Artificial Sequence
  Description of Artificial Sequence Synthetic oligonucleotide
atcatgacctcctcacctgt

SEQ ID NO: 112 - DNA, 20 bp, Artificial Sequence
  Description of Artificial Sequence Synthetic oligonucleotide
aggaggtcatgatccccttc

SEQ ID NO: 113 - DNA, 20 bp, Artificial Sequence
  Description of Artificial Sequence Synthetic oligonucleotide
ggcactgcccacaggtgagg

SEQ ID NO: 114 - DNA, 20 bp, Artificial Sequence
  Description of Artificial Sequence Synthetic oligonucleotide
gtgccagatgaacttcccat

SEQ ID NO: 115 - DNA, 20 bp, Artificial Sequence
  Description of Artificial Sequence Synthetic oligonucleotide
tgccagatgaacttcccatt

SEQ ID NO: 116 - DNA, 20 bp, Artificial Sequence
  Description of Artificial Sequence Synthetic oligonucleotide
gccagatgaacttcccattg

SEQ ID NO: 117 - DNA, 20 bp, Artificial Sequence
  Description of Artificial Sequence Synthetic oligonucleotide
tctggcactgcccacaggtg

SEQ ID NO: 118 - DNA, 20 bp, Artificial Sequence
  Description of Artificial Sequence Synthetic oligonucleotide
ccagatgaacttcccattgg

SEQ ID NO: 119 - DNA, 20 bp, Artificial Sequence
  Description of Artificial Sequence Synthetic oligonucleotide
gttcatctggcactgcccac

SEQ ID NO: 120 - DNA, 20 bp, Artificial Sequence
  Description of Artificial Sequence Synthetic oligonucleotide
cccccaatgggaagttcatc

SEQ ID NO: 121 - DNA, 20 bp, Artificial Sequence
  Description of Artificial Sequence Synthetic oligonucleotide
aaataagaatgtcccccaat

SEQ ID NO: 122 - DNA, 20 bp, Artificial Sequence
  Description of Artificial Sequence Synthetic oligonucleotide
aaaataagaatgtcccccaa

SEQ ID NO: 123 - DNA, 20 bp, Artificial Sequence
  Description of Artificial Sequence Synthetic oligonucleotide
cacaaacggaaacaatgcaa

SEQ ID NO: 124 - DNA, 20 bp, Artificial Sequence
  Description of Artificial Sequence Synthetic oligonucleotide
cctctgcttagaaaaagctg

SEQ ID NO: 125 - DNA, 20 bp, Artificial Sequence
  Description of Artificial Sequence Synthetic oligonucleotide
ccacagctttttctaagcag

SEQ ID NO: 126 - DNA, 20 bp, Artificial Sequence
  Description of Artificial Sequence Synthetic oligonucleotide
tcgattggtgaaggggaagg

SEQ ID NO: 127 - DNA, 20 bp, Artificial Sequence
  Description of Artificial Sequence Synthetic oligonucleotide
atctcgattggtgaagggga

SEQ ID NO: 128 - DNA, 20 bp, Artificial Sequence
  Description of Artificial Sequence Synthetic oligonucleotide
tttcatctcgattggtgaag

SEQ ID NO: 129 - DNA, 20 bp, Artificial Sequence
  Description of Artificial Sequence Synthetic oligonucleotide
gaaaaaagcatccaatcccg

SEQ ID NO: 130 - DNA, 20 bp, Artificial Sequence
  Description of Artificial Sequence Synthetic oligonucleotide
aaaagcatccaatcccgtgg

SEQ ID NO: 131 - DNA, 20 bp, Artificial Sequence
  Description of Artificial Sequence Synthetic oligonucleotide
gcatccaatcccgtggaggt

SEQ ID NO: 132 - DNA, 20 bp, Artificial Sequence
  Description of Artificial Sequence Synthetic oligonucleotide
tcccgtggaggttggcatcc

SEQ ID NO: 133 - DNA, 20 bp, Artificial Sequence
  Description of Artificial Sequence Synthetic oligonucleotide
tggcatccaggtcacgccag

SEQ ID NO: 134 - DNA, 20 bp, Artificial Sequence
  Description of Artificial Sequence Synthetic oligonucleotide
gatgccaacctccacgggat

SEQ ID NO: 135 - DNA, 20 bp, Artificial Sequence
  Description of Artificial Sequence Synthetic oligonucleotide
acctggatgccaacctccac

SEQ ID NO: 136 - DNA, 20 bp, Artificial Sequence
  Description of Artificial Sequence Synthetic oligonucleotide
gacctggatgccaacctcca

SEQ ID NO: 137 - DNA, 20 bp, Artificial Sequence
  Description of Artificial Sequence Synthetic oligonucleotide
cgtcatcctctggcgtgacc

SEQ ID NO: 138 - DNA, 20 bp, Artificial Sequence
  Description of Artificial Sequence Synthetic oligonucleotide
gataaacaatcgtcatcctc

SEQ ID NO: 139 - DNA, 20 bp, Artificial Sequence
  Description of Artificial Sequence Synthetic oligonucleotide
ctgctatgtgttcctgtttg

SEQ ID NO: 140 - DNA, 55 bp, Artificial Sequence
  Description of Artificial Sequence Synthetic primer
taatacgactcactatagggactcttagacataacacaccgttttagagctagaa

SEQ ID NO: 141 - DNA, 55 bp, Artificial Sequence
  Description of Artificial Sequence Synthetic primer
taatacgactcactatagggctcttagacataacacaccagttttagagctagaa

SEQ ID NO: 142 - DNA, 55 bp, Artificial Sequence
  Description of Artificial Sequence Synthetic primer
taatacgactcactatagggtacagagccccagtcctggagttttagagctagaa

SEQ ID NO: 143 - DNA, 55 bp, Artificial Sequence
  Description of Artificial Sequence Synthetic primer
taatacgactcactatagggttgcttttatcacaggctccgttttagagctagaa

SEQ ID NO: 144 - DNA, 55 bp, Artificial Sequence
  Description of Artificial Sequence Synthetic primer
taatacgactcactatagggttttatcacaggctccaggagttttagagctagaa

SEQ ID NO: 145 - DNA, 55 bp, Artificial Sequence
  Description of Artificial Sequence Synthetic primer
taatacgactcactatagggtttatcacaggctccaggaagttttagagctagaa

SEQ ID NO: 146 - DNA, 55 bp, Artificial Sequence
  Description of Artificial Sequence Synthetic primer
taatacgactcactatagggcacgcccccaccctaatcaggttttagagctagaa

SEQ ID NO: 147 - DNA, 55 bp, Artificial Sequence
  Description of Artificial Sequence Synthetic primer
taatacgactcactatagggatcagaggccaaacccttccgttttagagctagaa

SEQ ID NO: 148 - DNA, 55 bp, Artificial Sequence
  Description of Artificial Sequence Synthetic primer
taatacgactcactatagggcacaggctccaggaagggttgttttagagctagaa

SEQ ID NO: 149 - DNA, 55 bp, Artificial Sequence
  Description of Artificial Sequence Synthetic primer
taatacgactcactataggggaagggtttggcctctgattgttttagagctagaa

SEQ ID NO: 150 - DNA, 55 bp, Artificial Sequence
  Description of Artificial Sequence Synthetic primer
taatacgactcactatagggaagggtttggcctctgattagttttagagctagaa

SEQ ID NO: 151 - DNA, 55 bp, Artificial Sequence
  Description of Artificial Sequence Synthetic primer
taatacgactcactataggggtttggcctctgattagggtgttttagagctagaa

SEQ ID NO: 152 - DNA, 55 bp, Artificial Sequence
  Description of Artificial Sequence Synthetic primer
taatacgactcactatagggtttggcctctgattagggtggttttagagctagaa

SEQ ID NO: 153 - DNA, 55 bp, Artificial Sequence
  Description of Artificial Sequence Synthetic primer
taatacgactcactatagggttggcctctgattagggtgggttttagagctagaa

SEQ ID NO: 154 - DNA, 55 bp, Artificial Sequence
  Description of Artificial Sequence Synthetic primer
taatacgactcactatagggtctgattagggtgggggcgtgttttagagctagaa

SEQ ID NO: 155 - DNA, 55 bp, Artificial Sequence
  Description of Artificial Sequence Synthetic primer
taatacgactcactatagggattagggtgggggcgtgggtgttttagagctagaa

SEQ ID NO: 156 - DNA, 55 bp, Artificial Sequence
  Description of Artificial Sequence Synthetic primer
taatacgactcactatagggttagggtgggggcgtgggtggttttagagctagaa

SEQ ID NO: 157 - DNA, 55 bp, Artificial Sequence
  Description of Artificial Sequence Synthetic primer
taatacgactcactatagggtgggtggggtagaagaggacgttttagagctagaa

SEQ ID NO: 158 - DNA, 55 bp, Artificial Sequence
  Description of Artificial Sequence Synthetic primer
taatacgactcactataggggcaaacggccaccgatggaggttttagagctagaa

SEQ ID NO: 159 - DNA, 55 bp, Artificial Sequence
  Description of Artificial Sequence Synthetic primer
taatacgactcactatagggatcggtggccgtttgcccaggttttagagctagaa

SEQ ID NO: 160 - DNA, 55 bp, Artificial Sequence
  Description of Artificial Sequence Synthetic primer
taatacgactcactatagggctccctcccaggatcctctcgttttagagctagaa

SEQ ID NO: 161 - DNA, 20 bp, Artificial Sequence
  Description of Artificial Sequence Synthetic primer
aaaaaagcaccgactcggtg

SEQ ID NO: 162 - DNA, 20 bp, Artificial Sequence
  Description of Artificial Sequence Synthetic primer
accctggaaaacagcctgac

SEQ ID NO: 163 - DNA, 20 bp, Artificial Sequence
  Description of Artificial Sequence Synthetic primer
cacacggcatggcatacaaa

SEQ ID NO: 164 - DNA, 20 bp, Artificial Sequence
  Description of Artificial Sequence Synthetic primer
caccctggaaaacagcctga

SEQ ID NO: 165 - DNA, 21 bp, Artificial Sequence
  Description of Artificial Sequence Synthetic primer
caccttatattcccagggccg

SEQ ID NO: 166 - DNA, 21 bp, Artificial Sequence
  Description of Artificial Sequence Synthetic primer
cctaggacgcaccattctcac

SEQ ID NO: 167 - DNA, 20 bp, Artificial Sequence
  Description of Artificial Sequence Synthetic primer
attgggtctaacccccacct

SEQ ID NO: 168 - DNA, 20 bp, Artificial Sequence
  Description of Artificial Sequence Synthetic primer
tcagtgaaacgcaccagaca

SEQ ID NO: 169 - DNA, 20 bp, Artificial Sequence
  Description of Artificial Sequence Synthetic primer
aaccccagcacttaagcaaa

SEQ ID NO: 170 - DNA, 20 bp, Artificial Sequence
  Description of Artificial Sequence Synthetic primer
ggaggtcatgatccccttct

SEQ ID NO: 171 - DNA, 18 bp, Artificial Sequence
  Description of Artificial Sequence Synthetic primer
gccctggagaggatgttc

SEQ ID NO: 172 - DNA, 18 bp, Artificial Sequence
  Description of Artificial Sequence Synthetic primer
ttcttgccgtggccctta

SEQ ID NO: 173 - DNA, 18 bp, Artificial Sequence
  Description of Artificial Sequence Synthetic primer
cagtgcaggctgcctatc

SEQ ID NO: 174 - DNA, 20 bp, Artificial Sequence
  Description of Artificial Sequence Synthetic primer
atacttgtgggccagggcat

SEQ ID NO: 175 - DNA, 20 bp, Artificial Sequence
  Description of Artificial Sequence Synthetic primer
tgggtcatttcacagaggag

SEQ ID NO: 176 - DNA, 21 bp, Artificial Sequence
  Description of Artificial Sequence Synthetic primer
catcttccacattcaccttgc

SEQ ID NO: 177 - DNA, 20 bp, Artificial Sequence
  Description of Artificial Sequence Synthetic primer
acccagaagactgtggatgg

SEQ ID NO: 178 - DNA, 20 bp, Artificial Sequence
  Description of Artificial Sequence Synthetic primer
ttcagctcagggatgacctt

SEQ ID NO: 179 - DNA, 20 bp, Artificial Sequence
  Description of Artificial Sequence Synthetic primer
cttcgacccaagcaacatgc

SEQ ID NO: 180 - DNA, 20 bp, Artificial Sequence
  Description of Artificial Sequence Synthetic primer
cggtgagtgtcaggataggc

SEQ ID NO: 181 - DNA, 22 bp, Artificial Sequence
  Description of Artificial Sequence Synthetic primer
gccagaaaagagatatggcatc

SEQ ID NO: 182 - DNA, 20 bp, Artificial Sequence
  Description of Artificial Sequence Synthetic primer
agagagccttccgaaagagg

SEQ ID NO: 183 - DNA, 23 bp, Artificial Sequence
  Description of Artificial Sequence Synthetic primer
gttctctcttcttcctgacagtg

SEQ ID NO: 184 - DNA, 23 bp, Artificial Sequence
  Description of Artificial Sequence Synthetic primer
gaggtccctatgaaaagatggct

SEQ ID NO: 185 - DNA, 20 bp, Artificial Sequence
  Description of Artificial Sequence Synthetic primer
gttgaatgccaagtgcccaa

SEQ ID NO: 186 - DNA, 20 bp, Artificial Sequence
  Description of Artificial Sequence Synthetic primer
ggtctcagttcagccccttc

SEQ ID NO: 187 - DNA, 23 bp, Artificial Sequence
  Description of Artificial Sequence Synthetic primer
tgaacaatattgccttttgtgct

SEQ ID NO: 188 - DNA, 20 bp, Artificial Sequence
  Description of Artificial Sequence Synthetic primer
atgctgctgtaaggcactgt

SEQ ID NO: 189 - DNA, 24 bp, Artificial Sequence
  Description of Artificial Sequence Synthetic primer
tccataaaacaatgttgaggtggg

SEQ ID NO: 190 - DNA, 22 bp, Artificial Sequence
  Description of Artificial Sequence Synthetic primer
gtcctccagttgatcctgaagt

SEQ ID NO: 191 - DNA, 21 bp, Artificial Sequence
  Description of Artificial Sequence Synthetic primer
atccaggggctttgagattga

SEQ ID NO: 192 - DNA, 23 bp, Artificial Sequence
  Description of Artificial Sequence Synthetic primer
tctccatccccgttgttaagtga

SEQ ID NO: 193 - DNA, 20 bp, Artificial Sequence
  Description of Artificial Sequence Synthetic primer
cacacacaaagcccttctgc

SEQ ID NO: 194 - DNA, 20 bp, Artificial Sequence
  Description of Artificial Sequence Synthetic primer
caccatatccagcctgtcgg

SEQ ID NO: 195 - DNA, 20 bp, Artificial Sequence
  Description of Artificial Sequence Synthetic primer
ctctggaaagcagggaccat

SEQ ID NO: 196 - DNA, 20 bp, Artificial Sequence
  Description of Artificial Sequence Synthetic primer
gcatgctaaccagcacactg

SEQ ID NO: 197 - DNA, 20 bp, Artificial Sequence
  Description of Artificial Sequence Synthetic primer
acagagctgggccaataacc

SEQ ID NO: 198 - DNA, 21 bp, Artificial Sequence
  Description of Artificial Sequence Synthetic primer
tgttcacatgggaaagcctca

SEQ ID NO: 199 - DNA, 20 bp, Artificial Sequence
  Description of Artificial Sequence Synthetic primer
gcttgtgtgcgtgtatggaa

SEQ ID NO: 200 - DNA, 25 bp, Artificial Sequence
  Description of Artificial Sequence Synthetic primer
gtagcacactaatacatggaaatga

SEQ ID NO: 201 - DNA, 23 bp, Artificial Sequence
  Description of Artificial Sequence Synthetic primer
taagatccaaacacccaatcacg

SEQ ID NO: 202 - DNA, 23 bp, Artificial Sequence
  Description of Artificial Sequence Synthetic primer
agtcttcagctggtgatttcagg

SEQ ID NO: 203 - DNA, 23 bp, Artificial Sequence
  Description of Artificial Sequence Synthetic primer
gtaccattcctccaactaaggca

SEQ ID NO: 204 - DNA, 24 bp, Artificial Sequence
  Description of Artificial Sequence Synthetic primer
ctaagttcttccacctcgagtcat

SEQ ID NO: 205 - DNA, 20 bp, Artificial Sequence
  Description of Artificial Sequence Synthetic primer
agctgagatcacaccactgc

SEQ ID NO: 206 - DNA, 20 bp, Artificial Sequence
  Description of Artificial Sequence Synthetic primer
tctagcccctggtaacctcc

SEQ ID NO: 207 - DNA, 22 bp, Artificial Sequence
  Description of Artificial Sequence Synthetic primer
acagaagatgaatagcggaaca

SEQ ID NO: 208 - DNA, 20 bp, Artificial Sequence
  Description of Artificial Sequence Synthetic primer
tagtgacgcgaatagccctg

SEQ ID NO: 209 - DNA, 20 bp, Artificial Sequence
  Description of Artificial Sequence Synthetic primer
atcagcctggatccctaccc

SEQ ID NO: 210 - DNA, 20 bp, Artificial Sequence
  Description of Artificial Sequence Synthetic primer
acctgagattcctctgggct

SEQ ID NO: 211 - DNA, 22 bp, Artificial Sequence
  Description of Artificial Sequence Synthetic primer
tctgtttcatggtgatgcttga

SEQ ID NO: 212 - DNA, 25 bp, Artificial Sequence
  Description of Artificial Sequence Synthetic primer
acaatttttcactcttggtactctt

SEQ ID NO: 213 - DNA, 25 bp, Artificial Sequence
  Description of Artificial Sequence Synthetic primer
tctctccttagatttcttcatgtct

SEQ ID NO: 214 - DNA, 20 bp, Artificial Sequence
  Description of Artificial Sequence Synthetic primer
cagaacaaaggcacagccac

SEQ ID NO: 215 - DNA, 22 bp, Artificial Sequence
  Description of Artificial Sequence Synthetic primer
acagaatgctatgagagacgtt

SEQ ID NO: 216 - DNA, 22 bp, Artificial Sequence
  Description of Artificial Sequence Synthetic primer
ggtaggaaaaacgcagaaaaga

SEQ ID NO: 217 - DNA, 20 bp, Artificial Sequence
  Description of Artificial Sequence Synthetic primer
catgacttggtgaagcccct

SEQ ID NO: 218 - DNA, 20 bp, Artificial Sequence
  Description of Artificial Sequence Synthetic primer
atttgggccaccaacttagg

SEQ ID NO: 219 - DNA, 22 bp, Artificial Sequence
  Description of Artificial Sequence Synthetic primer
gtgtagtggaggagacagacaa

SEQ ID NO: 220 - DNA, 21 bp, Artificial Sequence
  Description of Artificial Sequence Synthetic primer
tttcaaacttgccagacccca

SEQ ID NO: 221 - DNA, 22 bp, Artificial Sequence
  Description of Artificial Sequence Synthetic primer
gttcccagcatcccaaaagaac

SEQ ID NO: 222 - DNA, 21 bp, Artificial Sequence
  Description of Artificial Sequence Synthetic primer
ggaggctctgagagaatgagg

SEQ ID NO: 223 - DNA, 25 bp, Artificial Sequence
  Description of Artificial Sequence Synthetic primer
gatgtgttcaatgcaatggagatta

SEQ ID NO: 224 - DNA, 22 bp, Artificial Sequence
  Description of Artificial Sequence Synthetic primer
ggctgcctctcattctcttgta

SEQ ID NO: 225 - DNA, 25 bp, Artificial Sequence
  Description of Artificial Sequence Synthetic primer
agagtccaataaattgaggtttcac

SEQ ID NO: 226 - DNA, 20 bp, Artificial Sequence
  Description of Artificial Sequence Synthetic primer
ataactccattccgggagcc

SEQ ID NO: 227 - DNA, 20 bp, Artificial Sequence
  Description of Artificial Sequence Synthetic primer
cagggcctcacttttgcctc

SEQ ID NO: 228 - DNA, 22 bp, Artificial Sequence
  Description of Artificial Sequence Synthetic primer
tgactgcatatccatgcaccat

SEQ ID NO: 229 - DNA, 20 bp, Artificial Sequence
  Description of Artificial Sequence Synthetic primer
cagctgaggctactgctgtt

SEQ ID NO: 230 - DNA, 21 bp, Artificial Sequence
  Description of Artificial Sequence Synthetic primer
tcttctgttcactctttggct

SEQ ID NO: 231 - DNA, 20 bp, Artificial Sequence
  Description of Artificial Sequence Synthetic oligonucleotide
cactgataggggtcgcggta

SEQ ID NO: 232 - DNA, 20 bp, Artificial Sequence
  Description of Artificial Sequence Synthetic oligonucleotide
cttgaccaatagccttgaca

SEQ ID NO: 233 - DNA, 20 bp, Artificial Sequence
  Description of Artificial Sequence Synthetic oligonucleotide
gtggggaaggggcccccaag

SEQ ID NO: 234 - DNA, 265 bp, Homo sapiens
gacactgaaggctgggcacagccttggggaccgctcacaggacatgcagcagtgtgtgccgacaactccctaccgcgacccctatcagtgccgaccaagcacacaagatgcacacccaggctgggctggacagaggggtcccacaagatcacagggtgtgccctgagaaggtggggagctcacagcctccaagcattgcatcatcctggtaccaggaaggcaatgggctgccccatacccacttcccttcctagaattggcctgg

SEQ ID NO: 235 - DNA, 229 bp, Homo sapiens
ttcattcccattgagaaataaaatccaattctccatcaccaagagagccttccgaaagaggcccccctgggcaaacggccaccgatggagaggtctgccagtcctcttctaccccacccacgcccccaccctaatcagaggccaaacccttcctggagcctgtgataaaagcaactgttagcttgcactagactagcttcaaagttgtattgaccctggtgtgttatgt

SEQ ID NO: 236 - DNA, 145 bp, Homo sapiens
atttcccttctgatatctacttgaactttcagataaaaaaaaaaaagcaagttgcagtaacatgttatgctacacaaagattagcatgaatatccaccctctttaaacagccaccccacaccctggagaagagcaaatgtgaagt

SEQ ID NO: 237 - DNA, 330 bp, Homo sapiens
ctgctatgtgttcctgtttggggcaaattcctctagatgacgttgataaacaatcgtcatcctctggcgtgacctggatgccaacctccacgggattggatgcttttttcatctcgattggtgaaggggaaggtggcttatccacagctttttctaagcagaggctgccattgcattgtttccgtttgtgctcgataaaaataagaatgtcccccaatgggaagttcatctggcactgcccacaggtgaggaggtcatgatccccttctggagctcccaacgggccgtggtctggttcatcatctgtaagaatggcttcaagaggctcgg

SEQ ID NO: 238 - DNA, 300 bp, Homo sapiens
gtggaactgctgaagggtgcttccttttattcttcatccctagccagccgccggcccctggcctcactggatactctaagactattggtcaagtttgccttgtcaaggctattggtcaaggcaaggctggccaacccatgggtggagtttagccagggaccgtttcagacagatatttgcattgagatagtgtggggaaggggcccccaagaggatactgctaattttttttatagcctttgccttgttccgattcagtcattccagtttttctctaatttattcttccctttagctagt

SEQ ID NO: 239 - DNA, 300 bp, Homo sapiens
gtggaactgctgaagggtgcttccttttattcttcatccctagccagccgccggcccctggcctcactggatactctaagactattggtcaagtttgccttgtcaaggctattggtcaaggcaaggctggccaacccatgggtggagtttagccagggaccgtttcagacagatatttgcattgagatagtgtggggaaggggcccccaagaggatactgctgcttaattttttttatagcctttgccttgttccgattcagtcattccaatttttctctaatttattcttccctttagc

SEQ ID NO: 240 - DNA, 20 bp, Artificial Sequence
  Description of Artificial Sequence Synthetic primer
gagagtgcagacaggggaag

SEQ ID NO: 241 - DNA, 23 bp, Artificial Sequence
  Description of Artificial Sequence Synthetic oligonucleotide
ctaacagttgcttttatcacagg

SEQ ID NO: 242 - DNA, 23 bp, Artificial Sequence
  Description of Artificial Sequence Synthetic oligonucleotide
ttgcttttatcacaggctccagg

SEQ ID NO: 243 - DNA, 23 bp, Artificial Sequence
  Description of Artificial Sequence Synthetic oligonucleotide
ttttatcacaggctccaggaagg

SEQ ID NO: 244 - DNA, 23 bp, Artificial Sequence
  Description of Artificial Sequence Synthetic oligonucleotide
tttatcacaggctccaggaaggg

SEQ ID NO: 245 - DNA, 23 bp, Artificial Sequence
  Description of Artificial Sequence Synthetic oligonucleotide
cacaggctccaggaagggtttgg

SEQ ID NO: 246 - DNA, 20 bp, Artificial Sequence
  Description of Artificial Sequence Synthetic oligonucleotide
ctaacacttgcttttatcac

SEQ ID NO: 247 - DNA, 22 bp, Artificial Sequence
  Description of Artificial Sequence Synthetic oligonucleotide
ttgcttttatcacacaggctcc

SEQ ID NO: 248 - DNA, 20 bp, Artificial Sequence
  Description of Artificial Sequence Synthetic oligonucleotide
ctgacttctatgcccagccc

SEQ ID NO: 249 - DNA, 17 bp, Artificial Sequence
  Description of Artificial Sequence Synthetic oligonucleotide
  modified_base (3)..(11): a, c, t, g, unknown or other
  misc_feature (3)..(11): This region may encompass 7-9 nucleotides
tgnnnnnnnnnwgatar